(19) United States
(12) Patent Application Publication
(10) Pub. No.: US 2007/0270350 A1
(43) Pub. Date: Nov. 22, 2007

(54) Methods and Compositions for the Prevention and Treatment of Inflammatory Diseases or Conditions

(75) Inventor: Inderjit Singh, Mount Pleasant, SC (US)

Correspondence Address: Michael R. Krawzsenek;FULBRIGHT & JAWORSKI L.L.P., 600 Congress Avenue, Suite 2400, Austin, TX, 78701, US

(73) Assignee: MUSC FOUNDATION FOR RESEARCH DEVELOPMENT, CHARLESTON, SC (US)

(21) Appl. No.: 10/596,198

(22) PCT Filed: Dec. 23, 2004
(86) PCT No.: PCT/US04/43432

§ 371 Date: Mar. 9, 2007

Related U.S. Application Data
(63) Continuation-in-part of application No. 60/531,828, filed on Dec. 23, 2003.
(63) Continuation-in-part of application No. 60/559,112, filed on Apr. 2, 2004.
(60) Provisional application No. 60/531,828, filed on Dec. 23, 2003.
(60) Provisional application No. 60/559,112, filed on Apr. 2, 2004.

(51) Int. Cl. A61K 38/05; A61P 19/02; A61P 19/08; A61P 3/04; A61P 3/10; A61K 31/7056; A61K 31/7008; A61K 31/537; A61K 31/401; A61K 31/366; A61K 31/22

(52) U.S. Cl. 514/18; 514/43; 514/237.8; 514/423; 514/460; 514/548; 514/62

(57) ABSTRACT

The present invention relates to methods and compositions of treating or preventing inflammatory diseases or conditions in a patient comprising administering to the patient a therapeutically effective amount of a composition comprising a glutathione donor, 5-amino 4-imidazolecarboxamide ribotide (AICAR), a 3-hydroxy-3-methylgluatryl-coenzymeA (HMG-CoA) reductase inhibitor, D-threo-1-Phenyl-2-decanoylamino-3-morpholino-1-propanol HCl (D-PDMP), and/or 1,5-(butylimino)-1,5-dideoxy-D-glucitol (Miglustat), or derivatives thereof.

This application claims the benefit of U.S. Provisional Application No. 60/559,112, filed Apr. 2, 2004, and U.S. Provisional Application No. 60/531,828, filed Dec. 23, 2003. These provisional applications are incorporated by reference.

### BACKGROUND OF THE INVENTION

A. Field of the Invention

The present invention relates generally to the field of biological sciences. More particularly, it concerns compositions and methods of their use for treating or preventing inflammatory diseases.

B. Description of Related Art

Inflammatory diseases and conditions pose serious health concerns on today's society. The current methods of treating such diseases and conditions can be overwhelming and financially burdensome on the patient and on the health care system as a whole. This situation is exasperated by the simple fact that there are a number of different kinds of inflammatory diseases ranging from stroke, Alzheimer's disease, Parkinson's disease, multiple sclerosis, viral encephalitis, acquired immunodeficiency disease (AIDS)-related dementia, amyotrophic lateral sclerosis, brain trauma, spinal cord disorders, and other neurodegenerative diseases.

For example, stroke is the third leading cause of death in the United States of America and is associated with serious long-term physical and cognitive disabilities, especially for elderly patients (Felgin et al. 2003; Sarti et al. 2000). A stroke is an event that produces localized reductions in blood flow to part of the brain. Nearly 85% of strokes are ischemic in nature, and under these conditions, oxygen-starved brain cells, mainly neurons in the ischemic center (core), quickly undergo necrosis due to ATP depletion and ionic failure. The core is surrounded by a ring-like penumbra (Leker and Shohami 2002), which, during a stroke is electrically silent but still has significant blood flow. Penumbral functions may be recovered with restoration (reperfusion) of blood supply within first several hours of stroke. Penumbral cell death occurs via apoptosis and is slow. Delayed oxygenation, apoptosis, and reperfusion contribute to the vulnerability of this area to inflammation and free radical attack. Ultimately, the injury leads to neurodegeneration and loss of brain functions. The rat model of focal cerebral ischemia using the middle cerebral artery occlusion (MCAO) technique is widely accepted as the best model for the clinical manifestations of stoke in the human (Ginsberg and Busto 1989).

In the central nervous system, apoptosis may play an important pathogenetic role in neurodegenerative diseases such as ischemic injury and white matter diseases (Thompson, 1995; Bredesen, 1995). Both X-linked adrenoleukodystrophy (X-ALD) and multiple sclerosis (MS) are demyelinating diseases with the involvement of proinflammatory cytokines in the manifestation of white matter inflammation. The presence of immunoreactive tumor necrosis factor a (TNF-α) and interleukin 1 (IL-1β) in astrocytes and microglia of X-ALD brain has indicated the involvement of these cytokines in immunopathology of X-ALD and aligned X-ALD with MS, the most common immune-mediated demyelinating disease of the CNS in man (Powers, 1995; Powers et al., 1992; McGuinnes et al., 1995; McGuiness et al., 1997). Several studies demonstrating the induction of proinflammatory cytokines at the protein or mRNA level in cerebrospinal fluid and brain tissue of MS patients have established an association of proinflammatory cytokines (TNF-α, IL-13, IL-2, IL-6, and IFN-γ) with the inflammatory loci in MS (Maimone et al., 1991; Tsukada et al., 1991; Rudick and Ransohoff, 1992).

X-linked adrenoleukodystrophy (X-ALD), an inherited, recessive peroxisomal disorder, is characterized by progressive demyelination and adrenal insufficiency (Singh, 1997; Moser et al., 1984). It is the most common peroxisomal disorder affecting between 1/15,000 to 1/20,000 boys and manifests with different degrees of neurological disability. The onset of childhood X-ALD, the major form of X-ALD, is between the age of 4 to 8 and then death within the next 2 to 3 years. Although X-ALD presents as various clinical phenotypes, including childhood X-ALD, adrenomyeloneuropathy (AMN), and Addison's disease, all forms of X-ALD are associated with the pathognomonic accumulation of saturated very long chain fatty acids (VLCFA) (those with more than 22 carbon atoms) as a constituent of cholesterol esters, phospholipids and gangliosides (Moser et al., 1984) and secondary neuro inflammatory damage (Moser et al., 1995). The necrologic damage in X-linked adrenoleukodystrophy may be mediated by the activation of astrocytes and the induction of proinflammatory cytokines. Due to the presence of similar concentration of VLCFA in plasma and as well as in fibroblasts of X-ALD, fibroblasts are generally used for both prenatal and postnatal diagnosis of the disease (Singh, 1997; Moser et al., 1984).

The deficient activity for oxidation of lignoceroyl-CoA ligase as compared to the normal oxidation of lignoceroyl-CoA in purified peroxisomes isolated from fibroblasts of X-ALD indicated that the abnormality in the oxidation of VLCFA may be due to deficient activity of lignoceroyl-CoA ligase required for the activation of lignoceric acid to lignoceroyl-CoA (Hashmi et al., 1986; Lazo et al., 1988). While these metabolic studies indicated lignoceroyl-CoA ligase gene as a X-ALD gene, positional cloning studies led to the identification of a gene that encodes a protein (ALDP), with significant homology with the ATP-binding cassette (ABC) of the super-family of transporters (Mosser et al., 1993). The normalization of fatty acids in X-ALD cells following transfection of the X-ALD gene (Cartier et al., 1995) supports a role for ALDP in fatty acid metabolism; however, the precise function of ALDP in the metabolism of VLCFA is not known at present.

Similar to other genetic diseases affecting the central nervous system, the gene therapy in X-ALD does not seem to be a real option in the near future and in the absence of such a treatment a number of therapeutic applications have been investigated (Singh, 1997; Moser, 1995). Adrenal insufficiency associated with X-ALD responds readily with steroid replacement therapy, however, there is as yet no proven therapy for neurological disability (Moser, 1995). Addition of monoenoic fatty acid (e.g., oleic acid) to cultured skin fibroblasts of X-ALD patents causes a reduction of saturated VLCFA presumably by competition for the same chain elongation enzyme (Moser, 1995). Treatment of X-ALD patients with trioleate resulted in 50% reduction of VLCFA. Subsequent treatment of X-ALD patients with a mixture of trioleate and trieruciate (popularly known as Lorenzo's, oil) also led to a decrease in plasma levels of VLCFA (Moser, 1995; Rizzo et al., 1986; Rizzo et al., 1989). Unfortunately, the clinical efficacy has been unsatisfactory since no proof of favorable effects has been observed by attenuation of the myelinolytic inflammation in X-ALD patients (Moser, 1995). Moreover, the exogenous addition of unsaturated VLCFA induces the production of superoxide, a highly reactive oxygen radical, by human neutrophils (Hardy et al., 1994). Since cerebral demyelination of X-ALD is associated with a large infiltration of phagocytic cells to the site of the lesion (Powers et al., 1992), treatment with unsaturated fatty acids may even be toxic to X-ALD patients. Bone marrow therapy also appears to be of only limited value because of the complexity of the protocol and of insignificant efficacy in improving the clinical status of the patient (Moser, 1995).

Experimental allergic encephalomyelitis (EAE) is an inflammatory demyelinating disease of the central nervous system (CNS) that serves as a model for the human demyelinating disease, multiple sclerosis (MS). Studies have shown that the majority of the inflammatory cells constitute of T-lymphocytes and macrophages (Merrill and Benveniste, 1996). These effector cells and astrocytes have been implicated in the disease pathogenesis by secreting number of molecules that act as inflammatory mediators and/or tissue damaging agents such as nitric oxide (NO). NO is a molecule with beneficial as well as detrimental effects. In neuroinflammatory diseases like EAE, high amounts of NO produced for longer durations by inducible nitric oxide synthase (iNOS) acts as a cytotoxic agent towards neuronal cells. Previous studies have shown NO by itself or it's reactive product (ONOO⁻) may be responsible for death of oligodendrocytes, the myelin producing cells of the CNS, and resulting in demyelination in the neuroinflammatory disease processes (Merrill et al., 1993; Mitrovic et al., 1994).

Infiltrating T-lymphocytes in EAE produce pro-inflammatory cytokines such as IL-12, TNF-α and IFN-γ (Merrill and Benveniste, 1996). In addition to T-cells and macrophages, astrocytes have also been shown to produce TNF-α (Shafer and Murphy, 1997). Convincing evidence exists to support a role for both TNF-α and IFN-γ in the pathogenesis of EAE (Taupin et al., 1997; Villarroya et al., 1996; Issazadeh et al., 1995). Investigations with antibodies against TNF-α have shown that in mice these antibodies protect against active and adaptively transferred EAE disease (Klinkert et al., 1997). The expression of TNF-α and IFN-γ during EAE disease could result in the upregulation of iNOS in macrophage and astrocytes because TNF-α and IFN-γ have been shown to be potent inducers of iNOS in macrophages and astrocytes in culture (Xie et al., 1994). This induction of iNOS could result in the production of NO, which if produced in large amounts may lead to cytotoxic effects. Peroxynitrite (ONOO⁻) has been identified in both MS and EAE CNS (Hooper et al., 1997; van der Veen et al., 1997). The role of peroxynitrite in the pathogenesis of EAE is supported by the beneficial effects of uric acid, a peroxynitrite scavenger, against EAE and by a subsequent survey documenting that MS patients had significantly lower serum uric acid levels than those of controls (Hooper et al., 1998). However, aggravation of EAE by inhibitors of NOS activity (Ruuls et al., 1996) and in an animal model of iNOS gene knockout (Fenyk-Melody et al., 1998) indicate that NO may not be the only pathological mediator in EAE disease process. In addition to NO other free radicals such as reactive oxygen intermediates (O₂⁻, H₂O₂, and OH⁻) can also be stimulated by cytokines (Merrill and Benveniste, 1996). Reactive oxygen intermediates (ROI) and NO are believed to be key mediators of pathophysiological changes that take place during inflammatory disease process. ROI's such as superoxide anion, hydroxy radicals and hydrogen peroxide can also be stimulated by TNF-α (Merrill and Benveniste, 1996). Therefore, it is likely that both the direct modulation of cellular functions by proinflammatory cytokines and toxicity of the ROI and reactive nitrogen species may play a role in the pathogenesis of EAE disease.

Several studies on protein and/or mRNA levels in plasma, cerebrospinal fluid (CSF), brain tissue, and cultured blood leukocytes from MS patients have established an association of proinflammatory cytokines (TNF-α, IL-I and IFN-γ) with MS (Taupin et al., 1997; Villarroya et al., 1996; Issazadeh et al., 1995). The mRNA for iNOS has also been detectable in both MS as well as EAE brains (Bagasra et al., 1995; Koprowski et al, 1993). Semiquantitative RT-PCR™ for iNOS mRNA in MS brains shows markedly higher expression of iNOS mRNA in MS brains than control brains (Bagasra et al., 1995). Analysis of CSF from MS patients has also shown increased levels of nitrite and nitrate compared with normal control (Merrill and Benveniste, 1996). Peroxynitrite, ONOO⁻ is a strong nitrosating agent capable of nitrosating tyrosine residues of proteins to nitrotyrosine. Increased levels of nitrotyrosine have been found in demyelinating lesions of MS brains as well as spinal cords of mice with EAE (Hooper et al., 1998; Hooper et al, 1997). A strong correlation exists between CSF levels of cytokines, disruption of blood-brain barrier, and high levels of circulating cytokines in MS patients (Villarroya et al., 1996; Issazadeh et al., 1995). Increase in TNF-α and IFN-γ levels seems to predict relapse in MS and the number of circulating IFN-γ positive blood cells correlates with severity of disability. These observations support the view that in both MS and EAE, induction of proinflammatory cytokines and production of NO through iNOS play roles in the pathogenesis of these diseases.

Alzheimer's disease (AD) is a common degenerative dementia affecting primarily the elderly population. The disease is characterized by the decline of multiple cognitive functions and a progressive loss of neurons in the central nervous system. Deposition of beta-amyloid peptide has also been associated with AD. A number of investigators have noted that AD brains contain many of the classical markers of immune mediated damage. These include elevated numbers of microglia cells, which are believed to be an endogenous CNS form of the peripheral macrophage, and astrocytes. Of particular importance, complement proteins have been immunohistochemically detected in the AD brain and they most often appear associated with beta-amyloid containing pathological structures known as senile plaques (Rogers et al., 1992; Haga et al., 1993).

These initial observations which suggest the existence of an inflammatory component in the neurodegeneration observed in AD has been extended to the clinic. A small clinical study using the nonsteroidal anti-inflammatory drug, indomethacin, indicated that indomethacin significantly slowed the progression of the disease (Neurology, 43(8):1609 (1993)). In addition, a study examining age of onset among 50 elderly twin pairs with onsets of AD separated by three or more years, suggested that anti-inflammatory drugs may prevent or delay the initial onset of AD symptoms (Neurology, 44:227 (1994)).

Over the years numerous therapies have been tested for the possible beneficial effects against EAE or MS disease but with mixed results (Cross et al, 1994; Ruuls et al., 1996). Though aminoguandine (AG) has been described as a competitive inhibitor of iNOS and a suppressor of its expression (Corbett and McDaniel, 1996; Joshi et al., 1996), to date few compounds which inhibit iNOS are of potential therapeutic value have been identified. This deficiency is particularly troubling given the significant cellular damage which can arise as a result of iNOS-mediated nitric oxide toxicity, especially in chronic inflammatory disease states.

### SUMMARY OF THE INVENTION

The inventor has discovered that particular compounds can be used to treat or prevent inflammatory diseases in humans. These compounds include glutathione donors, 5-amino 4-imidazolecarboxamide ribotide (AICAR), Activators of AMP-activated kinase, HMG-CoA reductase inhibitors, D-threo-1-Phenyl-2-decanoylamino-3-morpholino-1-propanol HCl (D-PDMP), and/or 1,5-(butylimino)-1,5-dideoxy-D-glucitol (Miglustat). Derivatives of these compounds can also be used to treat or prevent inflammatory diseases.

One aspect of the present invention includes a method of preventing or treating an inflammatory disease or condition in a patient comprising administering to the patient a therapeutically effective amount of a glutathione donor, AICAR, an activator of AMP-activated kinase (a non-limiting example includes an HMG-CoA reductase inhibitor), D-PDMP, and/or 1,5-(butylimino)-1,5-dideoxy-D-glucitol, or derivatives thereof. In particular embodiments, the glutathione donor can be administered with AICAR, an HMG-CoA reductase inhibitor, D-PDMP, and/or 1,5-(butylimino)-1,5-dideoxy-D-glucitol. Non-limiting examples of glutathione donors include S-nitroglutathione (GSNO), L-2-oxo-thiazolidine 4-carboxylate (Procysteine), N-acetyl cysteine (NAC), and N-acetyl glutathione. In particular embodiments, it is contemplated that the glutathione donor is not GSNO. The HMG-CoA reductase inhibitor can be a statin. Non-limiting examples of statins that can be used with the present invention include atorvastatin, lovastatin, rosuvastatin, fluvastatin, pravastatin, simvastatin, or cerivastatin, or derivatives thereof. The glutathione donor, AICAR, an activator of AMP-activated kinase, an HMG-COA reductase inhibitor, D-PDMP, and/or Miglustat can be formulated in a pharmaceutically acceptable vehicle.

In non-limiting aspects, the glutathione donor may be comprised in a pharmaceutically acceptable composition. The AICAR, the HMG-COA reductase inhibitor, the D-PDMP, or the Miglustat may be comprised in a pharmaceutically acceptable composition. In another embodiment, the glutathione donor and the AICAR, the HMG-CoA reductase inhibitor, the D-PDMP, or the Miglustat, may be comprised in the same or separate compositions.

In particular aspects of the present invention, the glutathione donor can be administered to the patient before, during, and/or after AICAR, an HMG-CoA reductase inhibitor, D-PDMP, and/or 1,5-(butylimino)-1,5-dideoxy-D-glucitol is administered to the patient. Similarly, AICAR, an HMG-CoA reductase inhibitor, D-PDMP and/or 1,5-(butylimino)-1,5-dideoxy-D-glucitol can be administered to the patient before, during, and/or after the glutathione donor is administered to the patient.

The methods of the present invention can further include determining whether a patent is in need of the prevention or treatment. Determining whether a patient is in need of the prevention or treatment can comprise determining whether a patient is at risk for developing an inflammatory disease or condition. Determining whether a patient is at risk for developing an inflammatory disease or condition can include taking a family history or a patient history.

Non-limiting examples of inflammatory diseases or conditions that can be treated or prevented with the present invention, include stroke, X-adenoleukodystrophy (X-ALD), cancer, septic shock, adult respiratory distress syndrome, myocarditis, arthritis, an autoimmune disease, an inflammatory bowel disease, an inflammatory nervous system disease, an inflammatory lung disorder, an inflammatory eye disorder, a chronic inflammatory gum disorder, a chronic inflammatory joint disorder, a skin disorder, a bone disease, a heart disease, kidney failure, a chronic demyelinating disease, an endothelial cell disease, a cardiovascular disease, obesity, a common cold, lupus, sickle cell anemia, diabetes, eye conditions, intrauterine/systemic infection, brain development (e.g., cerebral palsy), herpes dementia, organ transplant/bypass disorders, or a neurodegenerative disease. The neurodegenerative disease can be, for example, Alzheimer's disease, Parkinson's disease, Landry-Guillain-Barre-Strohl syndrome, multiple sclerosis, viral encephalitis, acquired immunodeficiency disease (AIDS)-related dementia, amyotrophic lateral sclerosis, brain trauma, or a spinal cord disorder. In further embodiments, the methods also include administering a second therapy used to treat or prevent the inflammatory disease or condition.

Another aspect of the present invention includes a pharmaceutically acceptable composition comprising a glutathione donor, AICAR, AMP-activated kinase (e.g., an HMG-CoA reductase inhibitor), D-PDMP, and/or 1,5-(butylimino)-1,5-dideoxy-D-glucitol or derivatives thereof. In particular aspects, the glutathione donor is not GSNO. The compositions of the present invention can be formulated in a pharmaceutically acceptable vehicle or carrier. In particular aspects, the composition can include a glutathione donor and AICAR, a glutathione donor and an activator of AMP-activated kinase (e.g., an HMG-CoA reductase inhibitor), a glutathione donor and D-PDMP, or a glutathione donor and 1,5-(butylimino)-1,5-dideoxy-D-glucitol. Non-limiting examples of glutathione donors include S-nitroglutathione (GSNO), Procysteine, N-acetyl cysteine, or N-acetyl glutathione. The AMP-activated kinase can be a statin. The statin can be atorvastatin, lovastatin, rosuvastatin, fluvastatin, pravastatin, simvastatin, or cerivastatin. In particular aspects, the compositions of the present invention can include a glutathione donor, AICAR, an activator of AMP-activated kinase, an HMG-COA reductase inhibitor, D-PDMP, and/or Miglustat.

In another embodiments, there is provided a method of preventing or treating an inflammatory disease or condition in a patient comprising administering to the patient a therapeutically effective amount of a glutathione donor, 5-amino 4-imidazolecarboxamide ribotide (AICAR), a statin, D-PDMP, and/or derivatives thereof. The inventor also contemplates a pharmaceutically acceptable composition comprising a glutathione donor, 5-amino 4-imidazolecarboxamide ribotide (AICAR), a statin, and D-PDMP, or derivatives thereof. In particular embodiments, the glutathione donor is not GSNO.

Non limiting examples of derivatives include chemically modified compounds of a glutathione donor, AICAR, an AMP-activated kinase (e.g., an HMG-CoA reductase inhibitor), D-PDMP, and/or Miglustat that still retain the desired effects on treating or preventing inflammatory diseases or conditions. Such derivatives may have the addition, removal, or substitution of one or more chemical moieties on the parent molecule. Non-limiting examples of modifications may include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, hetero atoms such as N, S, or O can be substituted into the structure instead of a carbon atom. Such a derivative may be prepared by any method known to those of skill in the art. The properties of such derivatives may be assayed for their desired properties by any means described herein or known to those of skill in the art.

Other non-limiting aspects of the present invention include combining the compositions and methods above with one or more induction suppressors of nitric oxide synthase and/or a cytokine. Examples of such induction suppressors can be found, for example, in U.S. Pat. No. 6,551,800, which is specifically incorporated by reference. Non-limiting examples of inductions suppressors include N-acetyl cysteine, Rolipram, Cilomilast, Roflumilast, forskolin, PDTC, and 4PBA. Additional compounds that can be used in combination, or alone, with the present invention include beta-interferons (non-limiting examples include betaseron, rebif, etc.), a monoclonal antibody, an inhibitor of the interaction between a proinflammatory cytokine and its receptor, an inhibitor of the interaction between TNF alpha and its receptor, Enbrel, Remicade, copaxone, Rituxan, an inhibitor IL-1 and its receptor, a T cell receptor or fragment thereof, a therapeutic vaccine, a capsase inhibitor, or a PDE-4 inhibitor can be used to treat an inflammatory disease or condition. Non-limiting examples of monoclonal antibodies that can be used with the present invention include antibodies against a proinflammatory cytokine, an inhibitor of the interaction between a proinflammatory cytokine and its receptor, a cell surface molecule or a cell surface receptor molecule, a T or B cell surface marker or idiotype, a TNF alpha molecule or a TNF alpha receptor, a cell surface marker on a cancer cell.

In yet another embodiment of the present invention, there is provided a method of preventing or treating an inflammatory disease or condition in a patient comprising administering to the patient a therapeutically effective amount of an induction suppressor of nitric oxide synthase and/or a cytokine. It is contemplated that additional compounds can also be administered with the induction suppressor. Non limiting examples of the additional compounds include the compounds discussed throughout the specification (e.g., beta-interferons a monoclonal antibody, an inhibitor of the interaction between a proinflammatory cytokine and its receptor, an inhibitor of the interaction between TNF alpha and its receptor, Enbrel, Remicade, copaxone, Rituxan, an inhibitor IL-1 and its receptor, a T cell receptor or fragment thereof, a therapeutic vaccine, a capsase inhibitor, or a PDE-4 inhibitor). Compositions of the present invention can include any combination of these compounds.

“Analogs” may include structural equivalents or mimetics.

A “patient” or “subject” may be an animal. Preferred animals are mammals, including but not limited to humans, pigs, cats, dogs, rodents, horses, cattle, sheep, goats and cows. Preferred patients and subjects are humans.

The terms “inhibiting,” “reducing,” “treating,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. Glycosphingolipids regulate the LPS-induced iNOS gene expression and NO production in primary astrocytes. Effect of D-PDMP (10, 25 and 50 uM) on NO production (A) and the induction of iNOS mRNA and protein expression (B) was examined after 6 hrs (for iNOS mRNA level) or 24 hrs (for iNOS protein and NO levels) of LPS/IFN [symbol] (1 ug/ml; 10 U/ml) treatment. The cells were pretreated with D-PDMP for 0.5 hr before LPS/IFN [symbol] treatment. The effect of LacCer on D-PDMP mediated inhibition of iNOS gene expression in astrocytes was also examined. The cells were pretreated with D-PDMP (50 [symbol] M) and/or LacCer (5 and 10 [symbol] [symbol] M) for 0.5 hr before LPS/IFN [symbol] stimulation. NO production (C) and iNOS mRNA and protein levels were quantified, 6 hrs and 24 hr after LPS/IFN [symbol] stimulation, respectively (D). The nitrite levels were normalized with total protein quantity. Levels of GAPDH were used as an internal standard for mRNA levels. The procedures for measurement of mRNA and of protein and NO are described in Materials and Methods. Data are represented as ±S.D from 3 independent experiments. ***p<0.001 in (A and C) as compared with unstimulated control; % p<0.01 and #p<0.001 in (A) as compared with LPS/IFN [symbol] stimulated cells. #p<0.001 in (B) as compared with LPS/IFN [symbol] stimulated cells; @, % p<0.001 in (B) as compared with D-PDMP treated cells.

FIGS. 2A-2E. Effect of various metabolites of the glycosphingolipid pathway on D-PDMP mediated inhibition of LPS-induced iNOS gene expression. Primary astrocytes were pretreated with D-PDMP and/or Glucer (A), GalCer (B), GM1 (C), GM3 (D) and GD3 (E) all at individual concentrations of 5 and 10 uM concentrations for 0.5 hr prior to stimulation with LPS/IFN [symbol] . NO production was assayed at 24 hrs following LPS/IFN [symbol] stimulation as described in legend for FIG. 1.

FIGS. 3A-3D. The effect of LPS/IFN [symbol] stimulation on the intracellular LacCer biosynthesis. Primary astrocytes were treated with ^{14C}galactose overnight followed by washing off the excessive amount by PBS. Upon pretreatment with D-PDMP 0.5 hr before LPS/IFN [symbol] stimulation, cells were harvested at the time points indicated and LacCer was analyzed by HPTLC as described in Materials and Methods (A). The amount of LacCer was normalized with the total protein quantity. The enzyme activity of Lactosylceramide synthase (GalT-2) was assayed by an in vitro assay using cell lysates derived from cells stimulated with LPS/IFN [symbol] for various durations as shown (B). The enzyme assay is described in Materials and Methods. Enzyme activity was normalized for total protein quantity. For the knockdown of GalT-2 expression, the cells were transfected with GalT-2 antisense DNA oligomer or its sequence-scrambled DNA oligomer as described in Materials and Methods. At 2d after transfection the cells were stimulated with LPS/IFN [symbol] and NO production (C) and the protein and mRNA levels of iNOS (D) were measured. Data are represented as S.D of three independent experiments. ***p<0.001 in (A) and ***p<0.001 in (B) as compare with unstimulated control. ***p<0.001 in (C) as compared to stimulated, untransfected cells; #p<0.001 in (C) as compared to transfected cells without LacCer.

FIGS. 4A-4D. LacCer regulates the LPS-induced iNOS gene expression in C6 glioma cells. Cells were pretreated with increasing doses of D-PDMP (10, 25, 50 [symbol] [symbol] M) 0.5 hr before LPS-stimulation. LPS/IFN-α induced NO production, iNOS protein and mRNA levels are inhibited by increasing doses of D-PDMP (A). Pretreatment with LacCer and D-PDMP blunts the inhibition of LPS-induced NO production, iNOS protein and mRNA levels by D-PDMP examined at 6 hrs (for iNOS mRNA) and 24 hrs (for NO production and iNOS protein levels) as described in Materials and Methods. However, pretreatment with GluCer does not have the same effect as LacCer (B). Data are represented ±S.D of 3 independent experiments. ***p<0.001 in (A) as compared to unstimulated control; # p<0.001 in (A) as compared to stimulated cells; & p<0.001 in (B) as compared to stimulated cells and ***p<0.001 in (B) as compared to D-PDMP treated cells. Supplementation of exogenous LacCer, reverses the inhibition by NO production (C) as well as iNOS mRNA and protein expression (D) whereas GluCer had no effect.

FIGS. 5A-5D. The involvement of small GTPase Ras and ERK1/2 in LacCer mediated regulation of LPS-induced iNOS gene expression. Ras activation was examined using GST tagged Raf-1 Ras binding domain as described in Materials and Methods. Time course for Ras activation following LPS/IFN [symbol] stimulation (A). Following pretreatment with LacCer and/or D-PDMP (50 [symbol] [symbol] M) and followed by LPS/IFN [symbol] stimulation for 5 mins cell lysates were used to assay levels of activated Ras. Ras activity was normalized for total protein quantity. Detection of GST-Raf1 bound Ras by western blot and densitometry of the autoradiograph are shown (B). ERK1/2 involvement in LacCer mediated iNOS expression was tested by using a MEK1/2 inhibitor PD98059. Following pretreatment for 0.5 hr with PD98059 before stimulation with LPS, NO production and iNOS protein levels at 24 hrs following stimulation were assayed (C). Upon pretreatment of cells with LacCer and/or D-PDMP followed by stimulation with LPS/IFN [symbol] for 20 minutes, cell lysates were also examined for activated ERK1/2 levels by immunoblot as described in Materials and Methods (D).

FIGS. 6A-6C. Involvement of LacCer in LPS/IFNα-mediated NFκB activation and iNOS gene expression. 24 hrs after transiently transfection of cells with [symbol] B-luciferase gene construct cells were pre-treated with D-PDMP, 0.5 hr prior to stimulation with LPS/IFN [symbol] [symbol] [symbol] [symbol] The cellular luciferase activity was measured as described in Materials and Methods. Data are represented as ±SD of 3 independent experiments (A) The NF-κB DNA binding activity was detected by gel shift assay using 10 μg of nuclear extract from cells pretreated for 0.5 hr with LacCer and/or increasing doses of D-PDMP followed by stimulation with LPS/IFNα for 45 minutes (B). The cytoplasmic extract was used to detect the levels of phosphorylated I [symbol] B and total I [symbol] B levels by immunoblot using anti-phospho I [symbol] B antibodies (C).

FIG. 7A-7B. iNOS mRNA and protein expression at the site of injury following SCI. iNOS mRNA (A) and protein levels (B) were significantly greater than sham values in vehicle (VHC) treated rats. D-PDMP treated rats showed significantly lower mRNA and protein expression as compared with vehicle treated rats. Data are represented ±SD. ***p<0.001 in (A) as compared to VHC treated Sham; #p<0.001 as compared to VHC treated 12 hr.

FIGS. 8A-8L. Double immunofluorescence staining of spinal cord sections at the lesion epicenter for iNOS/GFAP co-expression. Immunofluorescent microscopy images of spinal cord sections from SCI rats, stained with antibodies to iNOS (green) and GFAP (red) as described in Materials and Methods. (A-C) shows GFAP (A), iNOS (B) and their overlap (C) in Vehicle treated Sham. (D-F) shows GFAP (D), iNOS (E) and their overlap (F) in VHC treated SCI. (G-I) shows GFAP (G), iNOS(H) and their overlap (I) in D-PDMP treated Sham. (J-L) shows GFAP (J), iNOS (K) and their overlap (L) in D-PDMP treated SCI rats.

FIGS. 9A-9L. Double immunofluorescence staining of spinal cord sections from site of injury for TUNEL positive nuclei and Neuronal nuclei (NeuN): Immunofluorescent images of spinal cord sections from SCI rats stained for TUNEL positive cells using APOPTAG detection kit and antibodies to a neuronal specific marker NeuN as described in Materials and Methods. (A-C) shows NeuN (A), TUNEL (B) and their overlap (C) in vehicle treated Sham. (D-F) shows NeuN (D), TUNEL (E) and their overlap (F) in vehicle treated SCI. (G-H) shows NeuN (G), TUNEL (H) and their overlap (I) in D-PDMP treated Sham. (J-L) shows NeuN (J), TUNEL (K) and their overlap (L) in D-PDMP treated SCI rats.

FIGS. 10A-10H. Histological and myelin content examination of spinal cord sections from the site of injury of SCI rats. (A-D) shows H&E examination of spinal cord sections from vehicle treated Sham (A), SCI (B) and D-PDMP treated Sham (C) and SCI (D). (E-H) shows LFB-PAS staining for myelin in vehicle treated Sham (E) and SCI (F) and D-PDMP treated Sham (G) and SCI (H).

FIG. 11. Schematic representation of the model for LacCer mediated regulation of LPS/IFNα-induced iNOS gene expression.

FIGS. 12A-12C. Representative two TTC stained brain sections (# 3 and # 4 out of the six consecutive from cranial to caudate regions) from each group (A), infarct volume (B) and neurological scores (C). Photographs are shown at 24 h of reperfusion demonstrating that administration of GSNO after the onset of MCAO (20 nm) reduces the infarction, infarct volume and improves neurological evaluation score. Saline treated ischemic brain sections (vehicle) showed infarction as non-stained white both in striatum and cortex areas. GSNO treated brain sections (GSNO) showed great improvement in staining, hence much less infarction and infarct volume (p<0.0001). Neurological evaluation score was recorded as described in Materials and Methods. GSNO treatment decreased the evaluation score from 2.7 (in vehicle) to 1.1 (p<0.0001). The results are from 7 different experiments (n=7 in each group).

FIG. 13. Photomicrographs of immunohistochemistry of rat brain at 24 h of reperfision after 20 min MCAO. Enhanced reaction (brown DAB staining) shows higher expression of TNF-, IL-1 and iNOS in untreated (vehicle) than treated (GSNO) animals. TUNEL assay shows significant cell death in untreated (vehicle) than treated (GSNO) animals. (Magnification 400×).

FIGS. 14A-14B. Western blot of rat brain at 24 h of reperfusion after 20 min MCAO. Representative Western blot from three different set of experiments show expression of iNOS present in ipsilateral hemisphere of untreated animals (vehicle). Sham operated animals (sham) and treated animals (GSNO) did not show any significant expression of iNOS present in the brain (A). (B) is a graphic presentation of the result shown in (A).

FIGS. 15A-15F. Photomicrographs of immunohistochemistry of rat brain at 24 h of reperfusion after 20 min MCAO. Sham operated animals (sham) did not show staining (A) for ED 1, a marker for activation of macrophage/microglia. ED 1 expression (brown) was enhanced in untreated (vehicle) animals (B). Treatment with GSNO (C) reduced the expression of ED1. (Magnification 400×). GFAP staining showed the presence of significant number of activated astrocytes in vehicle (E), green fluorescence). GSNO treated animals had reduced number of activated astrocytes (F). Sham animals had no activated astrocytes present (D). (Magnification 400×).

FIGS. 16A-16L. The expression of iNOS in GFAP and ED I positive cells and colocalization of TUNEL and neurons at 24 h of reperfusion after 20 min MCAO. Immunostaining for iNOS (B) and GFAP (C) in a penumbral section are colocalized and are yellowish (A). Not all GFAP positive cells did not colocalize with iNOS. Similarly, iNOS (E-H) and ED1 (F and I) in a section from the penumbra ipsilateral are colocalized (D and G). Areas of colocalization in the combined image appear yellow. TUNEL positive cells (K) and NSE (neuron marker) positive cells (L) in a section from penumbra region are colocalized (J). (Magnification (A-I) 400× and (J) L200X).

FIG. 17. Caspase-3 activity in rat brain at 24 h of reperfusion after 20 min MCAO. Caspase-3 activity in cytosolic fraction of rat brain homogenates was measured as described in Materials and Methods. Caspase-3 activity was increased significantly in untreated (vehicle) animals. Treated animal (GSNO) had basal activity as in sham operated (sham) animals.

FIGS. 18A-18D. GSNO inhibits the LPS or LPS/IFNy-mediated iNOS gene expression in primary astrocytes and microglial cell line (BV2). Primary rat astrocytes and microglial cell BV2 were incubated for 30 min with different concentrations of GSNO as indicated, followed by LPS (1 mg/ml) or LPS/IFN [symbol] (1 mg/50 U/ml) treatment for 24 h. For detection of iNOS protein expression by immunoblot, cell lysate from astrocytes (A) or BV2 (C) was prepared and iNOS band was detected with iNOS antibody as mentioned in Materials and Methods. Blots are representative of two different experiments. Primary astrocytes (B) and BV2 (D) were transiently transfected with 1.50 g of iNOS-luciferase with lipofectamine 2000 (for primary astrocytes) or lipofectamine Plus (for BV2) according to the manufacturers instructions, followed by stimulation for 6 h with indicated treatment with GSNO and LPS or LPS/IFN [symbol] . Data are mean ±SD of three different values.

FIGS. 19A-19F. GSNO inhibits the LPS or LPS/IFNy-mediated NF-κB reporter activity in primary astrocytes and microglial cell line (BV2). (A) Primary astrocytes and BV2 (D) were transiently transfected with 1.5 μg of p(NF-κB)3LdLuc with lipofectamine 2000 (for primary astrocytes) or lipofectamine Plus (for BV2) according to the manufacturers instructions, followed by stimulation for 4 h with indicated treatment with GSNO and LPS or LPS/IFNα. Data are mean ±SD of three different values. (B) Primary astrocytes and BV2 (E) were transiently co-transfected with 1.5 [symbol] g of p(NF- [symbol] B)3LdLuc along with 0.5 mg of p65 and p50 and 0. L mg of pCMV- [symbol] -gal/well. Treatment of cells and luciferase activity was performed as described earlier. Data are mean ±SD of three experiments. Primary astrocytes (C) and BV2 (F) were transiently co-transfected with 1.5 μg of iNOS-luciferase along with 0.5 mg of p65 and p50 and 0.1 mg of pCMV- [symbol] [symbol] -gal/well. Treatment of cells and luciferase activity was performed as described earlier. Data are mean ±SD of three experiments. In all co-transfection studies, total DNA was kept constant (total 2.5 mg/well) and to normalize total DNA pcDNA3 (Invitrogen) was used. To normalize transfection efficiency, 0.1 mg of pCMV- [symbol] -gal/well were transfected and ( [symbol] -galactosidase activity was detected by [symbol] -galactosidase assay kit (Invitrogen)

FIGS. 20A-20C. AICAR inhibits LPS-induced cytokine synthesis in a dose-dependent manner. Primary rat astrocytes (A), primary microglia (B) and peritoneal macrophages (C) were incubated for 2 h with different concentrations of AICAR as indicated, followed by LPS (1 μg/ml) treatment for 24 h. The inventor measured the concentration of TNFα (left), IL-1β (center) and IL-6 (right) released in the medium using ELISA. For TNFα levels, media was taken out at 6 h of LPS treatment while for IL-1β and IL-6 at 24 h. Results are the mean ±SD of four determinations. *p<0.001 as compared to LPS treatment, #p<0.001 as compared to control.

FIGS. 21A-21E. AICAR inhibits the expression of iNOS in primary astrocytes, microglia and peritoneal macrophages. NO was measured in supernatant of primary astrocytes, microglia (A) and peritoneal macrophages (B) after 24 h of LPS/AICAR treatment. Data are mean ±SD of four different experiments. *p<0.001 as compared to LPS treatment, #0<0.001 as compared to control. For detection of iNOS protein expression by immuno blot in response to AICAR treatment, cell lysate from astrocytes was prepared after 24 h with LPS treatment (C). Blots are the representation of three different experiments. For detection of iNOS message, RNA was isolated from astrocytes 6 h after treatment with LPS and processed for northern blot analysis as mentioned in “Materials and Methods” (D). *p<0.001 as compared to LPS treatment, #p<0.001 as compared to control. Blots are representatives of three different experiments. Primary astrocytes were transiently transfected with lipofectamine 2000 reagent with 1. [symbol] [symbol] g/well iNOS-luciferase reporter vector along with 0.1 [symbol] g/well of pCMV- [symbol] -gal. After 24 h of transfection, cells were pretreated with indicated concentration of AICAR for 2 h followed by LPS (10 g/ml) for 4 h. Cells were lysed and processed for luciferase activity (Promega) and [symbol] -galactosidase (Invitrogen). Luciferase activity was normalized with respect to [symbol] -galactosidase activity and expressed relative to the activity of the control. Data are mean ±SD of three different values. ***p<0.001 as compared to control, @ p<0.001 as compared to LPS treatment (e). Primary astrocytes were transiently transfected as mentioned before and cells were treated with GGPP (10 [symbol] M), FPP (10 [symbol] M), mevalonate (10 mM), AICAR (1 mM) and LPS (1 [symbol] gml⁻¹) as indicated and luciferase activity were determined (f). Results are mean ±SD of three different values. ***p<0.001 as compared to control, #p<0.001 as compared to LPS treatment, NS, not significant as compared to LPS treatment, !p>0.05 (not significant) as compared to LPS/AICAR treatment.

FIG. 22. AICAR inhibits NO production and iNOS gene expression in glial cells via activation of AMPK: Primary astrocytes were pretreated with AICAR (1 mM) for 2 h followed by [¹⁴C]-acetate pulse for 2 h. Lipids were isolated and incorporation of labeled acetate in cholesterol and fatty acids was assayed by HP-TLC (a). Data are mean ±SD of three different values. ***p<0.001 as compared to untreated cells. Inhibitors of adenosine kinase (5′-iodotubercidin, and IC-51, 0.1 μM) were preincubated for 30 min before the addition of AICAR (1 mM). After 2 h incubation with AICAR, primary astrocytes were processed for the detection of p-AMPK [symbol] [symbol] [symbol] p-Thr 172 [symbol] [symbol] AMPK [symbol] [symbol] [symbol] p-ACC and [symbol] actin (for equal loading) by immuno blot as mentioned in “Materials and Methods” (b). Densitometry analysis was performed to estimate the ratio of p-AMPK [symbol] and AMPK [symbol] or p-ACC and [symbol] actin. Blots are representative of two different experiments. The expression of iNOS protein was determined in cell lysate at 24 h in astrocytes, after treating cells 5′-iodotubercidin/IC-51/AICAR with or without LPS ( [symbol] [symbol] g/ml) (c). Blots are representative of two different experiments. Primary rat astrocytes were incubated for 48 h with an antisense or missense oligo (25 [symbol] M) along with oligofectamine transfection reagent and AMPK [symbol] levels were determined by immuno blot analysis (d-i). Cells were treated with LPS (1 [symbol] g/ml) and lysed for the detection of iNOS (ii) and AMPK [symbol] protein by immuno blot as mentioned before (d). Densitometry analysis was performed to estimate the ratio of AMPK [symbol] or iNOS and [symbol] actin. Blots are representatives of two different experiments. Microglial cells (BV2) were transiently transfected with lipofectamine Plus with iNOS-Luciferase with [symbol] -gal in the presence or absence of dominant negative AMPK [symbol] 2 (DN) (0.5 [symbol] [symbol] g/ml) as mentioned before pcDNA3 empty vector was used to normalize the total DNA content in cotransfection studies. After 48 h of transfection, cells were treated with AICAR (1 mM) and LPS (1 [symbol] g/ml) as indicated and luciferase activity were determined after 6 h of LPS stimulation (e). Luciferase activity was normalized with respect to [symbol] -galactosidase activity. Data are mean ±SD of three different values. ***p<0.001 as compared to control, #p<0.001 as compared to LPS treatment, !p<0.001 as compared to LPS/AICAR (0.5 mM) treatment, @p<0.01 as compared to LPS/AICAR (1 mM) treatment.

FIG. 23 AICAR inhibits LPS induced Mitogen Activated Protein Kinases (ERK1/2, p38 and JNK1/2) in primary astrocytes: Primary astrocytes were incubated with different concentration of AICAR (0.5 to 1 mM) for 2 h followed by LPS treatment (1 [symbol] g/ml) for 30 min. Cells were washed with chilled PBC and scraped in lysis buffer as mentioned in Methods and Material. 50 g of total protein was loaded on SDS-PAGE followed by immuno blot analysis with phosphor specific antibodies against p42/44, JNK1/2 and p38. Same blot was stripped and reprobed with pan antibodies of p42/44, JNK1/2 and p38 for equal loading. Blots are representative of two different experiments.

FIG. 24. AICAR inhibits LPS induced NF-κB transcriptional response in primary astrocytes and BV2 cells. Nuclear extract was prepared from LPS/AICAR treated primary astrocytes as indicated and analyzed by EMSA for NF-κB (a). EMSA data is representative of two different experiments. Microglial cells (BV2) were transiently co-transfected with 1.5 μg of p(NF-κB)₃L^{d}Luc along with 0.5 μg of AMPKα2 dominant negative or pcDNA3, followed by stimulation for 4 h with indicated treatment with AICAR (1 mM) and LPS (b). Luciferase activity was normalized with respect to [symbol] -galactosidase activity. Data are mean ±SD of three different values. ***p<0.001 as compared to control, #p<0.001 as compared to LPS treatment, !p<0.05 as compared to LPS/AICAR (0.5 mM) treatment, @p<0.05 as compared to LPS/AICAR (1 mM) treatment, NS, not significant as compared to LPS treatment. Immuno blot was performed for p65 and p50 in nuclear extract from primary astrocytes stimulated with LPS with or without AICAR (c). Blots are representative of two different experiments. Total cell lysate of primary astrocytes was processed for the detection of IkBα by immuno blot at indicated time period (d). Blots are representatives of two different experiments. Microglial cells (BV2) were transiently transfected with 1.5 μg of iNOS (−234/+31)-luciferase or iNOS (−331/+31NF- [symbol] B mutated)-luciferase followed by stimulation for 4 h with indicated treatment with AICAR (1 mM) and LPS (e). Luciferase activity was normalized with respect to [symbol] -galactosidase activity. Data are mean ±SD of four different values. ***p<0.001 as compared to control, #p<0.001 as compared to LPS treatment. NS: not significant as compared to control, @p<0.001 as compared to LPS treatment (iNOS (−234/+31)-luciferase transfected cells).

FIG. 25. AICAR inhibits LPS induced IKKα/ [symbol] activity and IKK [symbol] mediated NF- [symbol] B-luciferase activity in primary astrocytes and BV2 cells: Primary astrocytes cells were incubated with AICAR (1 mM) prior to LPS (1 [symbol] gml⁻¹). After 30 min, IKK [symbol] [symbol] [symbol] activity was measured as mentioned in “Materials and Methods.” Densitometry analysis was performed and expressed as arbitrary units (a). Data are mean ±SD of three different values. ***p<0.001 as compared to control, #p<0.001 as compared to LPS treatment. Microglial cells (BV2) and primary astrocytes were transiently co-transfected with 1.5 μg of p(NF-κB)₃L^{d}Luc along with 0.5 μg of HA-IKK [symbol] [symbol] or pcDNA3 and 0. [symbol] [symbol] g of pCMVA- [symbol] -gal/well. Luciferase and [symbol] -galactosidase activities were done as mentioned earlier (b & c). Data are mean ±SD of three experiments. ***p<0.001 as compared to control, #p<0.001 as compared to LPS treatment, !p<0.001 as compared to LPS treated and [symbol] [symbol] [symbol] [symbol] [symbol] transfected cells.

FIG. 26. AICAR inhibits LPS induced nuclear translocation of C/EBP by down regulating the expression of C/EBP- [symbol] . Nuclear extract were prepared from LPS/AICAR treated primary astrocytes as indicated and analyzed by EMSA for C/EBP (a). EMSA data is representative of two different experiments. Polyclonal IgGs specific for C/EBP- [symbol] , - [symbol] , - [symbol] and - [symbol] were used in supershift experiments with nuclear extracts from LPS-treated (3 h) primary rat astrocytes and the ³²P-labeled C/EBP oligomer. Autoradiograms are representative of two independent experiments performed on separate preparations of nuclear extracts (b). Nuclear extracts prepared from various treatments were subjected to immuno blot for C/EBP- [symbol] and - [symbol] proteins (c). Primary astrocytes were incubated with LPS (1 μgml⁻¹) with or without treatment of 1 mM of AICAR. At the defined time, RNA was isolated for northern blot analysis for C/EBP- [symbol] and - [symbol] (d). Blots are representative of two different experiments. Microglial cells (BV2) were transiently transfected with 1.5 μg of iNOS (−1486/+145)-luciferase or iNOS-C/EBPdel-luciferase followed by stimulation for 4 h with indicated treatment with AICAR (1 mM) and LPS (e). Luciferase activity was normalized with respect to 0 galactosidase activity. Data are mean ±SD of four different values. ***p<0.001 as compared to control, #p<0.001 as compared to LPS treatment, *p<0.05 as compared to control, @p<0.05 as compared to LPS treatment, &p<0.01 as compared to LPS treatment (iNOS (−1486/+145)-luciferase transfected cells).

FIG. 27. AICAR inhibits the expression of pro-inflammatory mediators in serum and brain cerebral cortex of LPS injected rats. Rats were given saline i.p. with or without AICAR (100 mg/kg) 1 h before LPS administration (0.5 mg/kg). Blood and organs were taken out at 6 h after LPS injection. The levels of NO (i), TNF [symbol] (ii), IFN [symbol] (iii) and IL-1 [symbol] (iv) were measured in serum by ELISA (a) as mentioned in “Materials and Methods.” Results are the mean ±SD of six determinations. *p<0.001 as compared to LPS treatment, #p<0.001 as compared to control, NS, not significant. Immuno blot was performed for iNOS protein in peritoneal macrophages isolated at 6 h (b). For determination of expression of cytokines, spleen was isolated from treated rats and total RNA was isolated by Trizol reagent (Life Technologies) for gene array (Superarray) (c). Results are the representation of two independent experiments. The cerebral cortex was isolated from treated rats and total RNA was isolated as mentioned before. The expression of iNOS, TNFα, and IL-1β was examined by RT-PCR (d) as mentioned in “Materials and Methods.” Blot is representatives of two different experiments.

FIG. 28. Schematic diagram showing the involvement of various cell types (vascular and brain cells) and inflammatory mediators secreted by these cells in neuroinflammatory diseases.

FIG. 29. Efficacy of Simvastatin as Therapy for Multiple Sclerosis. Data given as average clinical scores where: 0=normal; 1=piloerection, 2=loss of tail tonicity, 3=hind leg paralysis; 4=paraplegia, 5=moribund.

FIG. 30A-F. Lovastatin inhibits the clinical symptoms of EAE. The mean clinical scores of the diseased animals are given in (A), (C), and (E) and weight measurements are given in (B), (D), and (F). (A) Active EAE was induced in SJL/J mice by immunization with myelin PLP₁₃₉₋₁₅₁ peptide in CFA. (C) and (E). Passive EAE was induced by adoptive transfer of myelin-PLP₁₃₉₋₁₅₁ sensitized T cells into recipient SJL/J mice. (A) and (C). The mice (six per group) were treated i.p. with t or 5 mg/kg lovastatin every day from days 0-60 after induction of EAE; (E) Lovastatin started on day 10. (A), (C), and (E) Lovastatin-treated mice developed significantly less (p<0.001) severe disease. (B), (D), and (F) Weights measured biweekly for active and passive EAE mice. Data are representative of three independent experiments with consistent results.

FIG. 31A-B. The histopathology of spinal cord sections from adoptive EAE and lovastatin-treated SJL/J mice prepared from the lumbar regions (six per mouse) and fixed in 10% buffered formalin. The tissues were embedded in paraffin and sectioned at 5-μm thickness. (A) The tissues were stained with H&E and are shown at ×100 magnification. (B) The cells from spinal cord were isolated and stained for CD4+ and MHC class II cells, acquired by FACS, and analyzed by CellQuest.

FIG. 32. Statistical analysis of infiltrating cells stained for DAPI. Quantification of the infiltrates show significant numbers of monocyte/macrophage and glial and inflammatory cells are present in the spinal cord of EAE animals as compared with both control and Lovastatin treated (LN) animals.

FIG. 33A-I. Shows immunofluorescent detection of ED1 and IL-1β (A-C), LFA-1 (D-F), and CD3 (G-I) in the lumbar region of the rat spinal cord. Double immunofluorescence staining of Lewis rat spinal cord sections (lumbar region) for IL-1β and ED1 expression shows an increase in EAE animals (B) when compared with control (A) or treated animals (C). Co-localization of IL-1β and ED1 shows up as yellow/orange in EAE (B) animals only. Control (A) and treated (C) animal spinal cord sections do not show co-localization.

FIG. 34A-I. Statins inhibit the expression of TNF-α, IFN-γ and iNOS in CNS of mice.

FIG. 35A-J. Induction of Th2 cytokines with lovastatin. (A), (C), (E), (G), and (I) DNL cells were isolated on day 10 from PLP₁₃₉₋₁₅₁ immunized SJL/J mice and cultured in vitro at 5×10⁶ cells/ml in the presence of PLP₁₃₉₋₁₅₁ (5 μg/ml) and lovastatin (10 and 20 μM). (B) (D), (F), (H), and (J) Naïve T cells (98% purified) isolated from lymph nodes were cultured in anti-CD3 and anti-CD28-precoated plates at 1×10⁶ cells/ml with lovastatin (10 and 20 μM). The supernatants were collected at 48 h (for IFN-γ and TNF-α) and 120 h (for IL-4, IL-5, and IL-10) for cytokine measurements. (A-D) IFN-γ and TNF-α are significantly reduced (p<0.0001) in both PLP-primed and naïve T cells. (E-J) IL-10, IL-5, and IL-4 are significantly increased (p<0.0001). The values are the means of triplicate determinations at each point, and the error bars represent +/−SD. Data are representative of four different experiments with consistent results.

FIG. 36A-F. Effect of lovastatin on the expression of GATA-3 and T-bet in Th1 and Th2 cells. GATA3 and T-bet were analyzed in vivo in PLP₁₃₉₋₁₅₁ specific and naïve cells. The DLN from immunized and lovastatin-treated mice were harvested on day 10 and analyzed by Western blot for T-bet (A) and GATA3 (B). PLP₁₃₉₋₁₅₁ specific cells were incubated with (10 and 20 μM) lovastatin for 48 h. (C) and (D) T-bet and GATA3 were analyzed by Western blot, and bands were scanned with a densitometer, and arbitrary units were plotted. Naïve T cells were stimulated with anti-CD3 and CD28 for 48 h in the presence of rmIL-12 or rm-IL-4 (10 ng/ml) and lovastatin (10 and 20 μM). Cells wee harvested and lysed, and 50 μg protein was resolved, blotted onto a membrane, and probed with anti-T-bet (E) and anti-GATA3 (F). Data are representative of three independent experiments with consistent results.

FIG. 37A-D. Lovastatin inhibits nuclear translocation of NF-κβ in stimulated T cells. Naïve T cells (98% purified) were pretreated for 2 h with different concentrations for lovastatin and stimulated with platebound anti-CD3 and CD28 (2 μg/ml) for 4 h (for NF-κβ) and 30 min for Iκβα). (A) The expression of NF-κβ was analyzed by gel-shift. (B) further inhibition was observed in a dose-dependent manner (5-50 μM). (C) The nuclear extract was prepared and nuclear translocation of NF-κβ was analyzed by TranSignal array. (D) For determination of pIκβα and Iκβα, T cells stimulated as described above were harvested, and cytosolic fractions were used for detection of pIκβα and Iκβα. The bands were scanned by densitometer, and the ratio of pIκβα/Iκβα was plotted. Data are representative of two independent experiments with consistent results.

FIG. 38A-F. Effects of lovastatin on the expression of GATA-3 and T-bet in Th1 and Th2 cells. GATA3 and T-bet were analyzed in vivo in PLP₁₃₉₋₁₅₁ immunized (100 μg/mouse) and lovastatin treated (2 and 5 mg/kg) mice and in vitro in PLP₁₃₉₋₁₅₁ specific and naïve T cells. The DLN from immunized and lovastatin treated mice were harvested on day 10 and analyzed by Western blot for T-bet (A) and GATA3 (B). PLP₁₃₉₋₁₅₁ specific T cells were incubated with 10 and 20 μM lovastatin for 48 h. (C) and (D) T-bet and GATA3 were analyzed by Western blot, bands were scanned, with a densitometer, and arbitrary units were plotted. Naïve T cells were stimulated with anti-CD3 and CD28 for 48 h in the presence of rmIL-12 or rmIL-4 (10 ng/ml) and lovastatin (10 and 20 μM). Cells were harvested and lysed, and 50 μg protein was resolved, blotted onto a membrane, and probed with anti-Tbet (E) and anti-GATA3 (F). Data are representative of three independent experiments with consistent results.

FIG. 39. Combined blood brain barrier (BBB) locomotor score of spinal cord injury (SCI) animals plotted in days after contusion injury and displayed as +/−SD (21 represents normal locomotion. 0 represents no observable movement.

FIG. 40. Immunofluorescence staining for infiltration of monocytes from the vessels into injured spinal cord.

FIG. 41. Immunofluorescence staining for reactive gliosis.

FIG. 42. Results of oligodendrocyte apoptosis in sham, untreated, and treated models.

FIG. 43. Therapeutic efficacy of antioxidant and antiinflammatory drugs in an animal stroke model (middle cerebral arterial occlusion).

FIG. 44. Therapeutic efficacy of antioxidant and antiinflammatory drugs in an animal stroke model (middle cerebral arterial occlusion).

FIG. 45. Experimental design for the use of statins as a therapeutic for kinic acid induced seizures (epilepsy model).

FIG. 46. Effect of atorvastatin on the KA-induced neuronal cell death in rat Hippocampus (cresyl vilot stain). Statins inhibited the hippocampal cell death induced KA in hippocampus. The rats were orally pre-treated (7 days before) with atorvastatin (LP; 10 mg/kg) prior to KA (10 mg/kg, i.p.). At 3 days after KA injection, neuronal cell death in hippocampus was examined using cresyl violet stain.

FIG. 47. Effect of atorvastatin on the KA-induced ED-1 expression in CA3 region. Atorvastatin inhibited the infiltration of macrophages induced by KA in hippocampus. The rats were orally pre-treated (7 days before) with atorvastatin (LP; 10 mg/kg) prior to KA (10 mg/kg, i.p.). At 3 days after KA injection, infiltration of macrophages in the CA1 and CA3 regions of the hippocampus was examined using immunofluorescent labeling against for ED-1, as a marker of monocytes.

FIG. 48A-B. Effect of atorvastatin on the KA-induced CA1 neuronal cell death in rat hippocampus (tunnel stain). Atorvastatin inhibited apoptosis induced by KA in hippocampus. The rats were orally pre-treated (7 days before) with atorvastatin (LP; 10 mg/kg) prior to KA (10 mg/kg, i.p.). At 3 days after KA injection, neuronal cell death in the CA1 (A) and CA3 (B) regions of the hippocampus was examined using TUNNEL assay.

FIG. 49. Effect of lovastatin on the KA-induced neuronal cell death in rat hippocampus (cresyl violt stain). Lovastatin inhibited neuronal cell death in hippocampus. The rats were orally pre-treated (7 days before) with lovastatin (Lov; 10 mg/kg) prior to KA (10 mg/kg, i.p.). At 3 days after KA injection, neuronal cell death in hippocampus was examined using cresyl violet stain.

FIG. 50A-C. Effect of atorvastatin on the KA-induced TNF-α, IL-1β, and iNOS expression in rat hippocampus. Atorvastatin inhibited the expression of inflammatory genes induced by KA in hippocampus. The rats were orally pre-treated (7 days before) with atorvastatin (LP; 10 mg/kg) prior to KA (10 mg/kg, i.p.). At 3 days after KA injection, the expression of inflammatory genes (TNF-α, IL-1β, and iNOS) in hippocampus using real time PCR. The expression of each gene was normalized with GAPDH expression.

FIG. 51A-C. Effect of lovastatin on the KA-induced TNF-α, IL-1β, and iNOS expression in rat hippocampus. Lovastatin inhibited the expression of inflammatory genes induced by KA in hippocampus. The rats were orally pre-treated (7 days before) with lovastatin (Lov; 10 mg/kg) prior to KA (10 mg/kg, i.p.). At 3 days after KA injection, the expression of inflammatory genes (TNF-α, IL-1β, and iNOS) in hippocampus using real time PCR. The expression of each gene was normalized with GAPDH expression.

FIG. 52A-D. Effects of avortastatin (LP) and lovastatin (lov) on the KA-induced seizure responses in rat. Seizure index: stage 1, facial clonus; stage 2, nodding; stage 3, forelimb clonus; stage 4, forelimb clonus with rearing; stage 5, rearing, jumping, and falling. The rats were orally pre-treated (7 days before) with lovastatin or atorvastatin (Lov or LP; 10 mg/kg) prior to KA (10 mg/kg, i.p.).

### DESCRIPTION OF ILLUSTRATIVE EMBODIMENTS

As previously noted, inflammatory diseases and conditions present numerous financial and health burdens on people inflicted with such diseases. Previous attempts to treat specific inflammatory diseases, such as stroke and Alzheimer's disease, have not been entirely successful. Moreover, the treatments can be harmful and debilitating to the patient.

The present invention discloses novel compositions and methods of their use for treating and preventing all kinds of inflammatory diseases and conditions. The compositions of the present invention may include any one of, or a combination of, a glutathione donor, AICAR, an activator of AMP-activated kinase, an HMG-COA reductase inhibitor, D-PDMP, and/or Miglustat. These and other aspects of the invention are described in greater detail below.

### A. Inflammatory Diseases

Inflammatory diseases or conditions that are contemplated as being treatable and/or preventable with the methods and compositions disclosed throughout this specification include, but are not limited to, psoriasis (Ruzicka et al., 1994; Kolb-Bachofen et al., 1994; Bull et al., 1994); uveitis (Mandia et al., 1994); type 1 diabetes (Eisieik & Leijersfam, 1994; Kroncke et al., 1991; Welsh et al., 1991); septic shock (Petros et al., 1991; Thiemermann & Vane, 1992; Evans et al., 1992; Schilling et al., 1993); pain (Moore et al., 1991; Moore et al, 1992; Meller et al., 1992; Lee et al., 1992); migraine (Olesen et al., 1994); rheumatoid arthritis (Kaurs & Halliwell, 1994); osteoarthritis (Stadler et al., 1991); inflammatory bowel disease (Miller et al., 1993; Miller et al., 1993); asthma (Hamid et al., 1993; Kharitonov et al., 1994); Koprowski et al., 1993); immune complex diseases (Mulligan et al., 1992); multiple sclerosis (Koprowski et al., 1993); ischemic brain edema (Nagafuji et al., 1992; Buisson et al., 1992; Trifiletti et al., 1992); toxic shock syndrome (Zembowicz & Vane, 1992); heart failure (Winlaw et al., 1994); ulcerative colitis (Boughton-Smith et al., 1993); atherosclerosis (White et al., 1994); glomerulonephritis (Muhl et al., 1994); Paget's disease and osteoporosis (Lowick et al., 1994); inflammatory sequelae of viral infections (Koprowski et al., 1993); retinitis, (Goureau et al., 1992); oxidant induced lung injury (Berisha et al., 1994); eczema (Ruzica et al., 1994); acute allograft rejection (Devlin, J. et al., 1994); and infection caused by invasive microorganisms which produce NO (Chen, Y and Rosazza, J. P. N., 1994).

Other inflammatory diseases discussed throughout the present specification and those known to a person of ordinary skill in the art are also contemplated as being treatable or preventable with the disclosed methods and compositions of the present invention.

### B. Glutathione Donors

Glutathione is a tri-peptide that includes the amino acids gamma-glutamic acid, cysteine, and glycine. Glutathione is also known as gamma-glutamylcysteinylglycine or GSH. GSH can be found in the human liver. Non-limiting examples of molecules that can act as a glutathione donor include L-2-oxo-thiazolidine 4-carboxylate (Procysteine), N-acetyl cysteine (NAC), N-acetyl glutathione, and S-nitroglutathione (GSNO). It is also contemplated by the present invention that any molecule that can carrier glutathione or that is or acts as a precursor to glutathione production can be used as a glutathione donor.

N-Acetyl Cysteine (NAC), for example, is the pre-acetylized form of the simple amino acid Cysteine. NAC is a known antioxidant and can be found naturally in foods. NAC is a an important precursor for glutathione synthesis in the body. L-2-oxo-thiazolidine 4-carboxylate (Procysteine) is a modified form of the amino acid cysteine. Procysteine plays a role in the synthesis of glutathione. N-acetyl glutathione acts as a carrier of glutathione.

GSNO is a physiological metabolite of glutathione (GSH and NO (Megson 2000; Schrammel et al. 2003), and is involved in several pharmacological activities (Chiueh 2002). GSNO reduces the frequency of embolic signals (Kaposzta et al. 2002a; Kaposzta et al. 2002b; Molloy et al. 1998) and can reverse acute vasocontriction and prevent ischemic brain injury after subarachnoid hemorrhage (Sehba et al. 1999). Furthermore, GSNO is at several fold more potent than GSH against oxidative stress (Rauhala et al. 1998) caused by ONOO⁻. GSNO can be a useful alternative to organic nitrates or tissue plasminogen activator (tPA); because it is endogenous, it may not produce tolerance.

GSNO is formed during the oxygen-dependent oxidation of NO in the presence of GSH. The decomposition of GSNO does not occur spontaneously and requires the presence of additional agents or enzymes including GSNO reductase or thioredoxin system (Steffen et. al. 2001; Zeng et al. 2001). Its degradation is also accelerated by the presence of thiol, ascorbate, or copper. GSNO and related S-nitrosothiols in the central nervous system are recognized to serve as signaling molecules between endothelial or astroglial cells and neurons (Chiueh and Rauhala 1999; Lipton 2001). S-nitrosothiol signaling mediated by GSNO is of central importance in the normal response to hypoxia (Lipton et al. 2001). GSNO is present in micromolar concentrations in the rat brain (Kluge et al. 1997). Furthermore, it has been suggested that protein S-nitrosylation/denitrosylation may serve as a component of an apoptotic (Gu et al. 2002) or another signaling pathway (Choi and Lipton 2000; Stamler et al. 1997).

### C. 5-amino 4-imidazolecarboxamide ribotide (AICAR)

5-Aminoimidazole-4-carboxamide ribonucleoside (AICAR) has been used extensively to activate AMPK in various cell types (Sullivan et al., 1994; Corton et al., 1995). Once inside the cell, AICAR is phosphorylated to ZMP and mimics the multiple effects of AMP on the allosteric activation of AMPK without altering the levels of nucleotides (Corton et al., 1995). It not only induces allosteric activation, but also promotes phosphorylation and activation of the upstream kinase, AMPK kinase (Moore et al., 1991; Sullivan et al., 1994). The expression of AMPK alpha 1 and alpha 2 catalytic subunits have been reported in the developing mouse brain (Turnley et al., 1999), but their function has yet to be explored.

It is contemplated that AICAR can be used alone, or in combination with the other compounds disclosed in the specification, to treat or prevent inflammatory diseases and conditions.

### D. HMG-CoA Reductase Inhibitors

HMG-CoA reductase catalyzes the conversion of hydroxymethylglutaryl-CoA to mevalonic acid, an early rate-limiting step in cholesterol biosynthesis. Particular HMG-CoA reductase inhibitors that can be used with the present invention include statins. In clinical studies, statins reduce total cholesterol, LDL cholesterol, apolipoprotein B and triglyceride levels. Statins can also increase HDL levels. Statins that are contemplated as being useful with the present invention include, but are not limited to, atorvastatin, lovastatin, rosuvastatin, fluvastatin, pravastatin, simvastatin, and cerivastatin. The chemical formulas for these statins include:

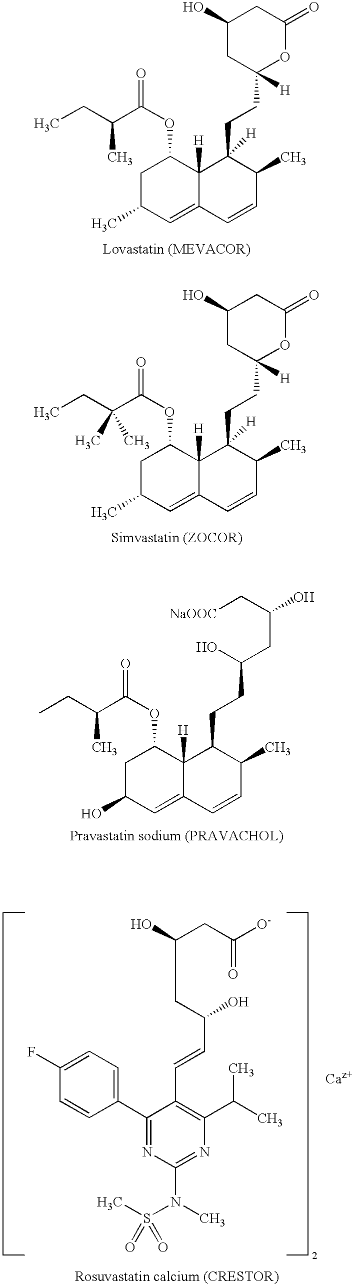

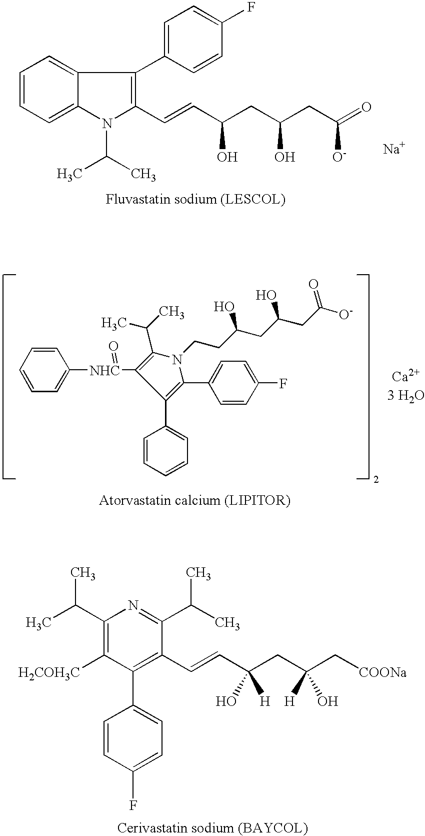

It is contemplated that HMG-CoA reductase inhibitors can be used alone, or in combination with the other compounds disclosed in the specification, to treat or prevent inflammatory diseases and conditions.

### E. D-threo-1-Phenyl-2-decanoylamino-3-morpholino-1-propanol HCl (D-PDMP)

D-PDMP is a glucosylceramide synthase and lactosylceramide synthase inhibitor. The molecular formula for D-PDMP is C₂₃H₃₈N₂₀₃HCl. D-PDMP includes a molecular weight of 427.1 and is soluble in water. The chemical formula for D-PDMP is:

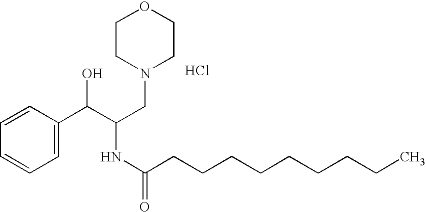

It is contemplated that D-PDMP can be used alone, or in combination with the other compounds disclosed in the specification, to treat or prevent inflammatory diseases and conditions.

### F. 1,5-(butylimino)-1,5-dideoxy-D-glucitol (Miglustat)

1,5-(butylimino)-1,5-dideoxy-D-glucitol (Miglustat) is an inhibitor of glucosylceramide synthase—a glucosyl transferase enzyme that plays a role in the synthesis of many glycosphingolipids. Miglustat is soluble in water. The molecular formula for Miglustat is C₁₀H₂₁NO₄ and has a molecular weight of 219.28. The chemical formula for Miglustat is:

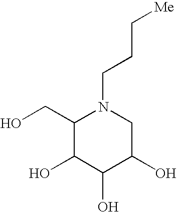

It is contemplated that Miglustat can be used alone, or in combination with the other compounds disclosed in the specification, to treat or prevent inflammatory diseases and conditions.

### G. Second Generation Compounds

In addition to the compounds described above, the inventor also contemplates that other sterically similar compounds may be formulated to mimic the key portions of these compounds. Such mimic compounds may be used in the same manner as a glutathione donor, AICAR, an activator of AMP-activated kinase, an HMG-CoA reductase inhibitor, D-PDMP, and/or Miglustat.

The generation of further structural equivalents or mimetics may be achieved by the techniques of modeling and chemical design known to those of skill in the art. The art of computer-based chemical modeling is now well known. Using such methods, a chemical compounds acting in a similar manner as a glutathione donor, AICAR, an activator of AMP-activated kinase, an HMG-CoA reductase inhibitor, D-PDMP, and/or Miglustat can be designed and synthesized. It will be understood that all such sterically similar constructs and second generation molecules fall within the scope of the present invention.

### H. Optimization in Therapy

A compound identified as having the ability to treat or prevent an inflammatory disease in a subject can be assayed by its optimum therapeutic dosage alone or in combination with another such compound. Such assays are well known to those of skill in the art, and include tissue culture or animal models for various disorders that are treatable with such agents.

Examples of such assays include those described herein and in U.S. Pat. No. 5,696,109. For instance, an assay to determine the therapeutic potential of molecules in brain ischemia (stroke) evaluates an agents ability to prevent irreversible damage induced by an anoxic episode in brain slices maintained under physiological conditions. An animal model of Parkinson's disease involving iatrogenic hydroxyl radical generation by the neurotoxin MPTP (Chiueh et al., 1992, incorporated herein by reference) may be used to evaluate the protective effects of iNOS or pro-inflammatory cytokine induction inhibitors. The neurotoxin, MPTP, has been shown to lead to the degeneration of dopaminergic neurons in the brain, thus providing a good model of experimentally induced Parkinson's disease (e.g., iatrogenic toxicity). An animal model of ischemia and reperfusion damage is described using isolated iron-overloaded rat hearts to measure the protective or therapeutic benefits of an agent. Briefly, rats receive an intramuscular injection of an iron-dextran solution to achieve a significant iron overload in cardiac tissue. Heart are then isolated and then subjected to total global normothermic ischemia, followed by reperfusion with the perfusion medium used initially. During this reperfusion, heart rate, and diastolic and systolic pressures were monitored. Cardiac tissue samples undergo the electron microscopy evaluation to measure damage to mitochondria such as swelling and membrane rupture, and cell necrosis. Comparison of measured cardiac function and cellular structural damage with or without the agent or iron-overloading after ischemia/reoxygenation is used to determine the therapeutic effectiveness of the agent.

### I. Purification Techniques

Various techniques suitable for use in purifying the compounds of the present invention will be well known to those of skill in the art. These include, for example, Polyacrylamide Gel Electrophoresis, High Performance Liquid Chromatography (HPLC), Gel chromatography or Molecular Sieve Chromatography and Affinity Chromatography. Examples of these and other techniques that can be used with the present invention can be seen in Sambrook et al., 2001.

The term “purified” as used herein, is intended to refer to a compound, isolatable from other compounds, wherein the compound is purified to any degree relative to its naturally-obtainable state. A purified compound, therefore, refers to a compound, free from the environment in which it may naturally occur.

### J. Pharmaceutical Composition and Routes of Administration

One embodiment of this invention includes methods of treating or preventing inflammatory diseases, by the delivery of anyone of a glutathione donor, AICAR, an AMP-activated kinase, an HMG-COA reductase inhibitor, D-PDMP, and/or Miglustat to a patient in need. These can compounds can be delivered in separate vehicles or can be comprised in one composition. An effective amount of the pharmaceutical compounds and compositions of the present invention, generally, is defined as that amount sufficient to detectably and repeatedly to ameliorate, reduce, minimize or limit the extent of the disease or its symptoms. More rigorous definitions may apply, including elimination, eradication or cure of disease.

1. Pharmaceutical Compositions

Pharmaceutical compositions of the present invention can include a glutathione donor, AICAR, an activator of AMP-activated kinase, an HMG-COA reductase inhibitor, D-PDMP, and/or Miglustat. The phrases “pharmaceutical or pharmacologically acceptable” refers to molecular entities and compositions that do not produce an adverse, allergic or other untoward reaction when administered to an animal, such as, for example, a human. The preparation of a pharmaceutical composition including a glutathione donor, AICAR, an activator of AMP-activated kinase, an HMG-COA reductase inhibitor, D-PDMP, and/or Miglustat will be known to those of skill in the art in light of the present disclosure, as exemplified by Remington's Pharmaceutical Sciences, 18th Ed. Mack Printing Company, 1990. Moreover, for animal (e.g., human) administration, it will be understood that preparations should meet sterility, pyrogenicity, general safety and purity standards as required by FDA Office of Biological Standards.

“Therapeutically effective amounts” are those amounts effective to produce beneficial results in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Except insofar as any conventional carrier is incompatible with the active ingredient, its use in the therapeutic or pharmaceutical compositions is contemplated.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The compositions of the present invention may comprise different types of carriers depending on whether it is to be administered in solid, liquid or aerosol form, and whether it need to be sterile for such routes of administration as injection.

The compositions may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target issue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments, the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments, the compositions are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition should be stable under the conditions of manufacture and storage, and preserved against the contaminating action of microorganisms, such as bacteria and fungi. It will be appreciated that exotoxin contamination should be kept minimally at a safe level, for example, less that 0.5 ng/mg protein.

2. Routes of Administration

The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrauterinely, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (Remington's, 1990).

### K. Combination Therapies

In order to increase the effectiveness of a treatment with the compositions of the present invention, such as composition comprising any combination of a glutathione donor, AICAR, an activator of AMP-activated kinase, an HMG-COA reductase inhibitor, D-PDMP, and/or Miglustat, it may be desirable to combine these compositions with other therapies effective in the treatment of inflammatory diseases or conditions.

The compositions of the present invention can precede or follow the other agent treatment by intervals ranging from minutes to weeks. It is contemplated that one may administer both modalities within about 12-24 h of each other and, more preferably, within about 6-12 h of each other. In some situations, it may be desirable to extend the time period for treatment significantly, where several days (2, 3, 4, 5, 6 or 7) to several weeks (1, 2, 3, 4, 5, 6, 7 or 8) lapse between the respective administrations.

Various combinations may be employed where a compositions including a composition contemplated by the present invention is “A” and the secondary agent, is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Material and Methods I

Reagents. Recombinant rat interferon gamma (IFN [symbol] [symbol] [symbol] and antibody against mouse macrophage iNOS was obtained from Calbiochem (CA). DMEM and FBS were from Life Technologies Inc. Lipopolysaccharide, (from Escherichia coli Serotype 0111:B4) was from Sigma (MO). Glucosylceramide, lactosylceramide, galactosylceramide, gangliosides and D-PDMP (C₂₃H₃₈N₂O₃.HCl; D-threo-1-Phenyl-2-decanoylamino-3-morpholino-1-propanol.HCl) were from Matreya Inc (PA). ¹⁴C-Galactose and ^{3H}UDP-Galactose was obtained from American Radiolabeled Chemicals (MO).

Cell Culture. Primary astrocyte-enriched cultures were prepared from the whole cortex of 1-day-old Sprague-Dawley rats as described earlier (Pahan et al., 1998). Briefly, the cortex was rapidly dissected in ice-cold calcium/magnesium free Hanks Balanced Salt Solution (HBSS) (Gibco, Grand Island, N.Y.) at pH 7.4 as described previously (Won et al., 2001). The tissue was then minced, incubated in HBSS containing trypsin (2 mg/ml) for 20 and washed twice in plating medium containing 10% FBS and 10 [symbol] g/ml gentamicin, and then disrupted by triturating through a Pasteur pipette following which cells were plated in 75-cm² culture flasks (Falcon, Franklin, N.J.). After incubation at 37° C. in 5% CO₂ for 1 day, the medium was completely changed to the culture medium (DMEM containing 5% FBS and 10 [symbol] g/ml gentamicin). The cultures received half exchanges with fresh medium twice a week. After 14-15 days the cells were shaken on the orbital shaker to remove the microglia at least 1 day and then seeded on multi-well tissue culture dishes. The cells were incubated with serum-free DMEM for 24 h prior to the incubation with drugs.

C6 rat glioma cells obtained from ATCC were maintained in Dulbecco's modified Eagle's medium (DMEM) (GIBCO, CA) containing 10% fetal bovine serum (FBS) (GIBCO) and 10 [symbol] g/ml gentamicin. All the cultured cells were maintained at 37° C. in 5% CO₂/95% air. At 80% confluency, the cells were incubated with serum free DMEM medium for 24 h prior to the incubation with LPS/IFN [symbol] and other chemicals.

Assay for NO production. Cells were cultured in 12-well plastic tissue culture plates. After the appropriate treatment, production of NO was determined by an assay of the culture supernatant for nitrite (Green et al., 1982). Briefly, 100 μl of culture supernatant was allowed to react with 100 μl of Griess reagent. The optical density of the assay samples was measured spectrophotometrically at 570 nm. Nitrite concentrations were calculated from a standard curve derived from the reaction of NaNO₂ in fresh media.

Western Blot Analysis. For iNOS protein, the cells were washed with cold Tris buffered saline (TBS; 20 mM Trizma base, and 137 mM NaCl, pH 7.5), lysed in 1×SDS sample loading buffer (62.5 mM Trizma base, 2% w/v SDS, 10% glycerol), following sonication and centrifugation at 1,5000×g for 5 min, the supernatant was used for the iNOS western immunoblot assay. The protein concentration of samples was determined with the detergent compatible protein assay reagent (Bio-Rad Laboratories, CA) using bovine serum albumin (BSA) as the standard. Sample was boiled for 3 min with 0.1 volumes of 10% O-mercaptoethanol and 0.5% bromophenol blue mix. Fifty μg of total cellular protein was resolved by electrophoresis in 8 or 12% polyacrylamide gels, electro-transferred to polyvinylidene difluoride (PVDF) filter and blocked with Tween 20 containing Tris-buffered saline [TBST; 10 mM Trizma base (pH 7.4), 1% Tween 20, and 150 mM NaCl] with 5% skim milk. After incubation with antiserum against iNOS (BD PharMingen, CA), or hRas (Upstate Biotechnology, CA), or phospho-specific MAPK (p42/44) (Signal Transduction), in PVDF buffer for 2 h at room temperature, the filters were washed 3 times with TBST buffer and then incubated with horseradish peroxidase conjugated anti-rabbit or mouse IgG for 1 h. The membranes were autoradiographed by using ECL-plus (Amersham Pharmacia Biotech) after washing with TBST buffer.

Nuclear Extraction and Electrophoretic mobility shift assay. Nuclear extracts from cells (1×10⁷ cells) were prepared using previously published method (Dignam et al., 1983) with slight modification. Cells were harvested, washed twice with ice-cold TBS, and lysed in 400 μl of buffer A containing, 10 mM KCl, 2 mM MgCl₂, 0.5 mM dithiothreitol, protease inhibitor cocktail (Sigma), and 0.1% Nonidet P40 in 10 mM HEPES, pH 7.9 for 10 min on ice. Following centrifugation at 5,000×g for 10 min, the pelleted nuclei were washed with buffer A without Nonidet P40, and re-suspended in 40 μl of buffer B containing 25% (v/v) glycerol, 0.42 M NaCl, 1.5 mM MgCl₂, 0.2 mM EDTA, 0.5 mM dithiothreitol, and Complete™ protease inhibitor cocktail (Roche) in 20 mM HEPES, pH7.9 for 30 min on ice. The lysates were centrifuged at 15,000×g for 15 min and the supernatants containing the nuclear proteins were stored at −70° C. until use. Ten μg of nuclear proteins was used for the electrophoretic mobility shift assay for detection of AP-1, NFκB, and C/EBP DNA binding activities. DNA-protein binding reactions were carried out at room temperature for 20 min in 10 mM Trizma base (pH 7.9), 50 mM NaCl, 5 mM MgCl₂, 1 mM EDTA, 1 mM dithiothreitol, 1 μg poly (dl-dC), 5% (v/v) glycerol, and approximately 0.3 pmol of NFκB (Santa Cruz Biotech) labeled with DIG-ddUTP using terminal deoxynucleotidyl transferase (Roche). Protein-DNA complexes were resolved from protein-free DNA in 5% polyacrylamide gels at room temperature in 50 mM Tris, pH 8.3, 0.38 M glycine, and 2 mM EDTA, and electroblotted onto positively charged nylon membranes. The chemiluminescence detection method for DIG-labeled probes is identical to the method described for the non-isotopic northern blot analysis in the preceding.

Transient Transfections and Reporter Gene Assay. 3×10⁵ cells/well were cultured in 6-well plates for one day before the transfection. Transfection was performed with plasmid concentration constant (2.5 μg/transfection) and 8 μl of Fugene transfection reagent (Roche Molecular Biochemicals). One day after transfection, the cells were placed in serum free media for overnight. Following appropriate treatment, the cells were washed with phosphate buffered saline (PBS), scrapped, and then resuspended with 100 μl of lysis buffer (40 mM of Tricine pH 7.8, 50 mM of NaCl, 2 mM of EDTA, 1 mM of MgSO₄, 5 mM of dithiothreitol, and 1% of Triton X-100). After incubation in room temperature for 15 min with occasional vortexing, the samples were centrifuged. The luciferase and β-galactosidase activities were measured by using luciferase assay kit (Stratagene, CA) and β-gal assay kit (Invitrogen, CA) respectively. The emitted light and optical absorbance was measured using Spectra Max/Gemini XG (Molecular Device, CA) and SpectraMax 190 (Molecular Device).

Quantification of Ras Activation. After stimulation, primary astrocytes in 6 well plates were washed with PBS and lysed in membrane lysis buffer (MLB; 0.5 ml of 25 mM HEPES, pH7.5, 150 mM NaCl, 1% Igepal CA-630, 0.25% sodium deoxycholate, 10% glycerol, 10 mM MgCl₂, 1 mM EDTA, 25 mM NaF, 1 mM of sodium orthovanadate, and EDTA free Complete™ protease inhibitor cocktail). After centrifugation (5,000×g) at 4° C. for 5 min, supernatant was used for Ras activation assay. One hundred μg of supernatant was used for binding with agarose conjugated Ras-binding domain (RBD) of Raf-1 which was expressed in BL21 (Invitrogen) Escherichia coli strain transformed by pGEX-2T-GST-RBD in the presence of 0.1 mM of IPTG as described previously (Herrmann et al., 1995). The binding reaction was performed at 4° C. for 30 min in MLB. Following washing with MLB three times, Ras-RBD complex were denatured by adding of 2×SDS sample buffer. Ras protein was identified by western immunoblot analysis with Ras antibodies from Upstate Biotechnology.

Measurement of lactosylceramide synthesis. Cultured cells were incubated in growth medium containing [¹⁴C] galactose (5 [symbol] Ci/ml) for 24 h as described previously. The medium was removed, and the cell monolayer was washed with sterile PBS to remove nonspecifically adsorbed radioactivity and fresh serum free medium was added. After the stimulation of cells with LPS/IFN [symbol] (1 [symbol] g/ml; 10 U/ml) for various durations cells were then harvested and washed with ice cold PBS and lysed by sonication. Following protein quantification, 2000 g of protein was used for extraction of lipids using Chloroform:Methanol:HCl (100:100:1). The organic phase was dried under nitrogen. Glycosphingolipids were resolved by high performance thin layer chromatography using chloroform/methanol/0.25% KCl (70:30:4, v/v/v) as the developing solvent. The chromatographic plate was dried in air and stained with iodine vapors. The gel area corresponding to LacCer was scraped, and radioactivity was measured employing “liquiscint” (NEN Life Science Products) as a scintillating fluid.

Identification and analysis of lactosylceramide purified from C6 cells. Lactosylceramide from LPS treated cells was resolved by a silica gel 60 TLC plate. Fatty acid methyl ester (FAME) was prepared as described earlier (Khan et al., 1998; Pahan et al., 1998). FAME was analyzed by gas chromatography (Shimadzu, GC 17A gas chromatograph) on silica capillary column and quantified as a percentage of total fatty acids identified. Mass spectrometry data were recorded as Finnegan LCQ classic (ion trap quadrupole) mass spectrometer.

GalT-2 activity assay. The activity of GalT-2 was measured using [³H]UDP-galactose as the galactose donor and GlcCer as the acceptor as described previously (Yeh et al., 2001). Briefly, cells were harvested in PBS and cell pellets were suspended in Triton X-100 lysis buffer. Cell lysates were sonicated and following protein quantification, 100 [symbol] g of cell lysate was added to reaction mixture containing 20 [symbol] M of cacodylate buffer (pH 6.8), 1 mM Mn/Mg, 0.2 mg/ml Triton X-100 (1:2 v/v), 30 nmol of GluCer and 0.1 mmol of UDP-[³H]galactose in a total volume of 100 [symbol] l. The reaction was terminated by adding 10 [symbol] l of 0.25M EDTA, 10 [symbol] l of 0.5M KCl and 500 [symbol] l of Chloroform/Methanol (2:1 v/v) and the products were separated by centrifugation. The lower phase was collected and dried under nitrogen. Following resolution on HPTLC plates, the gel was cut out and radioactivity was measured in a scintillation counter. Assay without exogenous GluCer served as blank and their radioactivity counts were subtracted from all respective data points.

Gal T-2 antisense oligonucleotides. A 20-mer antisense oligonucleotide of the following sequence (5′-CGCTTGAGCGCAGACATCTT-3′) targeted against rat lactosylceramide synthase (GalT-2) were synthesized by Integrated DNA Technology. A scrambled oligonucleotide (5′-CTGATATCGTCGATATCGAT-3′) was also synthesized and used as control. Cells were counted and plated a day before transfection and the following day were treated with Oligofectamine (Invitrogen)-oligonucleotide complexes (200 nM oligo) under serum free conditions. 2d following transfection the transfected cells were stimulated with LPS/IFN [symbol] (1 [symbol] g/ml) and levels of nitric oxide were checked 24 hr following stimulation. iNOS mRNA and protein levels were checked at 6 hrs and 24 hrs respectively, following stimulation of transfected cells.

RT-PCR amplification. Following total RNA extraction using TRIzol (GIBCO) as per manufacturer's protocol, single stranded cDNA was synthesized from total RNA. 5 [symbol] g total RNA was treated with 2U DNAse I (bovine pancreas, Sigma) for 15 min at room temperature in 18 ul volume containing 1×PCR buffer and 2 mM MgCl₂. It was then inactivated by incubation with 201 of 25 mM EDTA at 65° C. for 15 min; 2 [symbol] l of random primers were added and annealed to the RNA according to the manufacturer's protocol. cDNA was synthesized in a 50 [symbol] l reaction containing 5 [symbol] g of total RNA and 50-100U reverse transcriptase by incubating the tubes at 42° C. for 45 min. PCR amplification was conducted in 25 [symbol] l of reaction mixture with 1.0 [symbol] l of cDNA, 0.5 mM of each primer and under the manufacturer's Taq polymerase conditions (Takara, takara Shuzo Co. Ltd, Japan). The sequence of primers used for PCR amplification are as follows; iNOS, (Forward-5′ CTC CTT CM AGA GGC AAA AAT A 3′, Reverse-5′ CAC TTC CTC CAG GAT GTT GT 3′), GalT-2 (Forward-5′ TGG TAC MG CTA GAG GC 3′, Reverse-5′ GCA TGG CAC ATT GM C-3′), GAPDH (Forward-5′ CGG GAT CGT GGA AGG GCT MT GA 3′, Reverse-5′ CTT CAC GM GTT GTC ATT GAG GGC A3′). The PCR program included preincubation at 95° C. for 4 min, amplification for 30 cycles at 94° C. for 1 min plus 50° C. annealing for 1 min plus 74° C. extension for 1 min and a final 74° C. for 10 min extension. 10 ul of the PCR products were separated on 1.2% agarose gel and visualized under UV.

Real-time PCR. Total RNA isolation from rat spinal cord sections was performed using Trizol (GIBCO, BRL) according to the manufacturer's protocol. Real-time PCR was conducted using Biorad iCycler (iCycler iQ Multi-Color Real Time PCR Detection System; Biorad, Hercules, Calif., USA). Single stranded cDNA was synthesized from total RNA. 5 [symbol] g total RNA was treated with 2U DNAse I (bovine pancreas, Sigma) for 15 min at room temperature in 18 ul volume containing 1×PCR buffer and 2 mM MgCl₂. It was then inactivated by incubation with 2 μl of 25 mM EDTA at 65° C. for 15 min. The primer sets for use were designed (Oligoperfect™ designer, Invitrogen) and synthesized from Integrated DNA technologies (IDT, Coralville, Iowa, USA). The primer sequences for iNOS (forward) 5′-GM AGA GGA ACA ACT ACT GCT GGT-3′, iNOS (Reverse) 5′-GM CTG AGG GTA CAT GCT GGA GC, GAPDH (Forward) 5′-CCT ACC CCC MT GTA TCC GTT GTG-3′ and GAPDH (Reverse) 5′-GGA GGA ATG GGA GTT GCT GTT GAA-3′. IQTM SYBR Green Supermix was purchased from BIO-RAD (BIO-RAD Laboratories, Hercules, Calif.). Thermal cycling conditions were as follows: activation of DNA polymerase at 95° C. for 10 min, followed by 40 cycles of amplification at 95° C. for 30 sec and 58.3° C. for 30 sec. The normalized expression of target gene with respect to GAPDH was computed for all samples using Microsoft Excel data spreadsheet.

Induction of SCI in rats. Sprague-Dawley female rats (225-250 g weight) were purchased (Harlan laboratories, Durham, N.C.) for induction of SCI. All rats were given water and food pellets ad libitum and maintained in accordance with the ‘Guide for the Care and Use of Laboratory Animals’ of the US Department of Health and Human Services (National Institutes of Health, Bethesda, Md., USA). For the induction of SCI in rats a clinically relevant weight-drop device was used as described earlier (Gruner, 1992). Briefly, rats were anesthetized by intraperitoneal (i.p.) administration of ketamine (80 mg/kg) plus xylazine (10 mg/kg) followed by laminectomy at T12. While the spine was immobilized with stereotactic device, injury (30 g/cm force) was induced by dropping a weight of 5 gm from a height of 6 cm onto an impounder gently placed on the spinal cord. Sham operated animals underwent laminectomy only. Upon recovery from anesthesia, animals were evaluated neurologically and monitored for food and water intake. However, no prophylactic antibiotics or analgesics were used in order to avoid their possible interactions with the experimental therapy of SCI.

Treatment of SCI. Within 10 min after induction of SCI, rats received the glycosphingolipid inhibitor, D-PDMP (Matreya Inc, Pleasant Gap, Pa.). D-PDMP was dissolved in 5% Tween 80 in saline and diluted with sterile saline (0.85% NaCl) at the time of intraperitoneal (i.p.) administration to SCI rats. Animals (six per group) were randomly selected to form 4 different groups: vehicle (5% Tween 80 in saline) treated sham (laminectomy only) and SCI (5% Tween 80 in saline), and D-PDMP (20 mg/kg in 5% Tween 80) treated Sham and SCI. A single dose of D-PDMP was administered every 24 hrs after the first dose (which was given at 10 mins following SCI) until 72 hrs after injury. Animals were sacrificed under anesthesia 1 h, 4 h, 12 h, 24 h, 48 h and 72 h following treatment.

Preparation of spinal cord sections. Rats were anesthetized and sacrificed by decapitation. Spinal cord sections with the site of injury as the epicenter were carefully extracted from vehicle treated Sham and SCI as well as D-PDMP treated sham and SCI animals. Tissue targeted to be used for RNA and protein extraction was immediately homogenized in Trizol (GIBCO, BRL), snap frozen in liquid nitrogen and stored at −80° C. until further use. Total RNA was extracted as per manufacturer's protocol and used for cDNA synthesis as described earlier. Sections of spinal cord to be used for histological examination as well immunohistochemistry were fixed in 10% neutral buffered formalin (Stephens Scientific, Riverdale, N.J.). The tissues were embedded in paraffin and sectioned at 4- [symbol] M thickness.

Immunohistochemical analysis. Spinal cord sections were deparaffinized, sequentially rehydrated in graded alcohol percentages. Slides were then boiled in antigen unmasking fluids (Vector Labs, Burlingame, Calif.) for 10 minutes, cooled in the same solution for another 20 minutes and then washed 3× for 5 min each in Tris-sodium buffer (0.1 M Tris-HCL, pH-7.4, 0.15 M NaCl) with 0.05% Tween-20 (TNT). Sections were treated with Trypsin (0.1% for 10 min) and immersed for 10 min in 3% hydrogen peroxide to eliminate endogenous peroxidase activity. Sections were blocked in Tris sodium buffer with 0.5% blocking reagent (TNB) (supplied with TSA-Direct kit, NEN Life Sciences, Boston Mass.) for 30 min to reduce non-specific staining. For immunofluorescent double-labeling, sections were incubated overnight with anti-iNOS antibody (1:100, mouse monoclonal, Santa Cruz, Calif.) followed by antibodies against the astrocyte marker, GFAP (1:100, rabbit polyclonal, DAKO, Japan) for 1 hr. Anti-iNOS was visualized using fluorescein-isothiocyanate (FITC) conjugated anti-mouse IgG (1:100, Sigma) and GFAP using tetramethylrhodamine isothiocyanate (TRITC) conjugated anti-rabbit IgG (1:100, Sigma). The sections were mounted in mounting media (EMS, Fort Washington, Pa.) and visualized by immunofluorescence microscopy (Olympus) using Adobe Photoshop software. Rabbit polyclonal IgG was used as control primary antibody. Sections were also incubated with conjugated FITC anti-rabbit IgG (1:100, Sigma, St. Louis, Mo.), or TRITC conjugated IgG (1:100) without the primary antibody as negative control. H&E staining was carried out as described by (Kiernan, 1990). Luxol fast blue PAS was carried out according to (Lassmann and Wisniewski, 1979).

Fluorescent TUNEL assay. TUNEL assay was carried out using APOPTAG Fluorescein In Situ Apoptosis Detection Kit (Serological Corporation, Norcross, Ga.) according to manufacturer's protocol. For double labeling, sections were incubated with mouse anti-neuronal nuclei 1:100 (NeuN, Chemicon, USA). Sections were incubated with TRITC conjugated mouse IgG 1:100 (Sigma), mounted in mounting media and visualized by fluorescence microscopy.

Statistical analysis. The data was statistically analyzed by performing the Student Newman-Keuls test.

### Example 2

### Results I

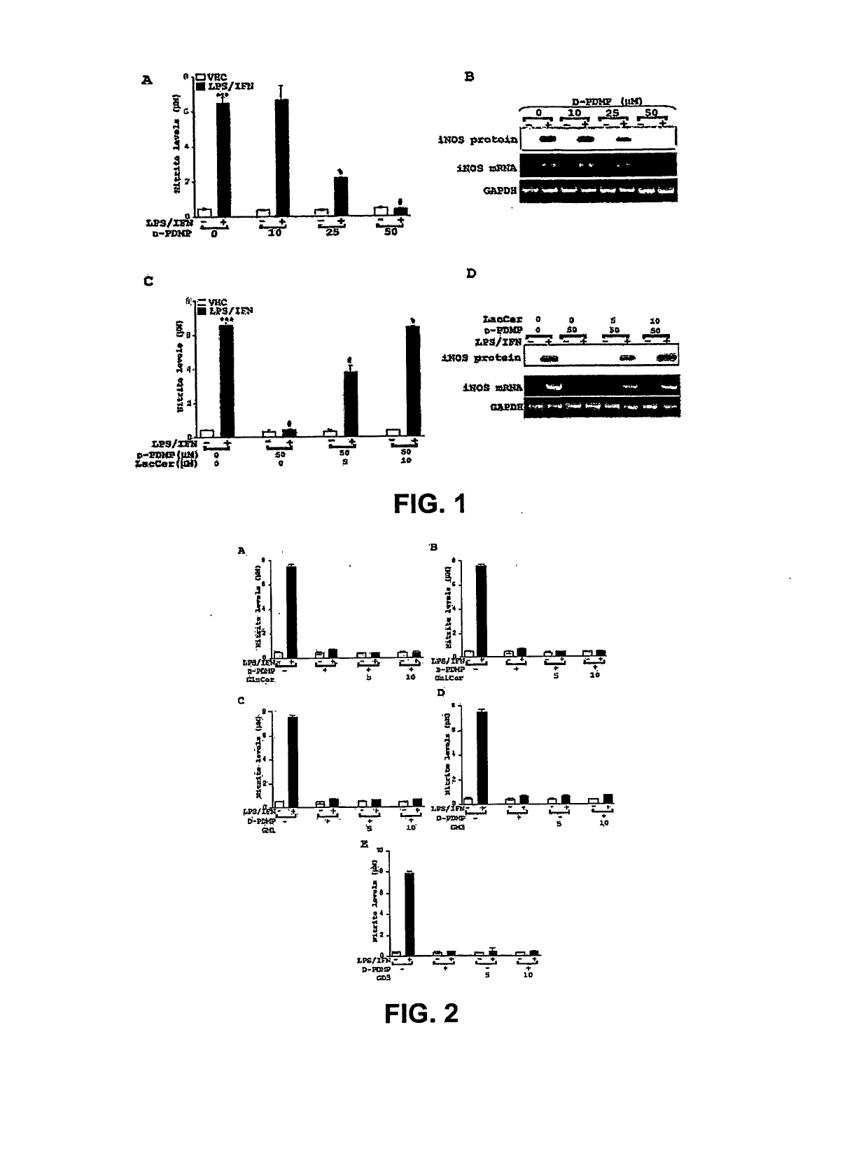

LPS/IFN [symbol] -induced NO production and iNOS gene expression is mediated by GSLs. LPS/IFN [symbol] stimulation of primary astrocytes resulting in iNOS gene expression is a complex multi-step process. The present study tested whether GSLs were somehow involved. Primary astrocytes pretreated for 0.5 h with several concentrations of the glycosphingolipid inhibitor D-PDMP (0, 10, 25 and 50 [symbol] M) followed by stimulation with LPS/IFN [symbol] [symbol] (1 [symbol] g/ml; 10 U/ml) showed a dose dependent decrease in production of nitric oxide (NO) (FIG. 1A) as well as mRNA and protein levels of iNOS (FIG. 1B). However, in the presence of increasing doses of LacCer, D-PDMP mediated inhibition of NO production (FIG. 1C) and iNOS gene expression (FIG. 1D) was blunted. To prove that this was a LacCer specific effect, other glycosphingolipid derivatives were also exogenously supplemented. However, the presence of Glucer (FIG. 2A), GalCer (FIG. 2B) and the various gangliosides-GM₁ (FIG. 2C), GM₃ (FIG. 2D) and GD₃ (FIG. 2E) did not reverse D-PDMP mediated inhibition of LPS/IFN [symbol] induced NO production as LacCer supplementation did. These studies indicate that a metabolite of the glycosphingolipid pathway, LacCer, may play a role in the regulation of LPS/IFN [symbol] mediated induction of iNOS gene expression and NO production.

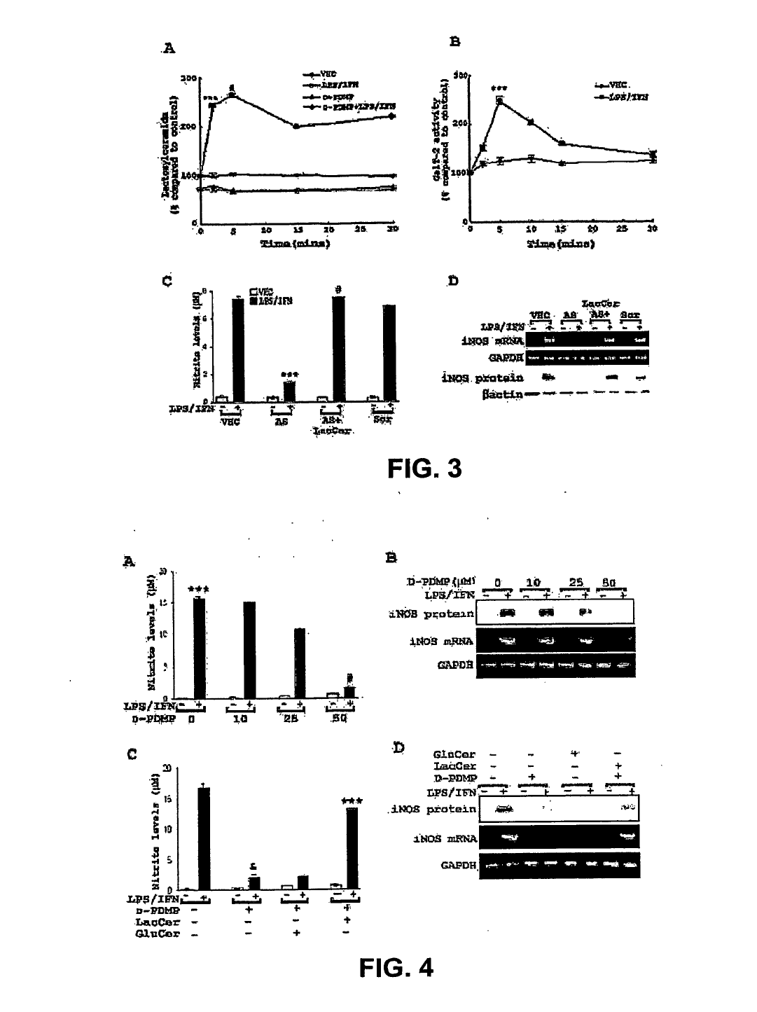

LPS/IFN [symbol] stimulation results in altered levels and lipid composition of lactosylceramide. To understand the mechanism of LPS/IFN [symbol] induced iNOS gene expression by LacCer the in situ levels of lactosylceramide were quantified. ¹⁴C labeled LacCer was resolved and characterized by Rf value using commercially available standard LacCer by high performance thin layer chromatography as described in Materials and Methods. As shown in FIG. 3A, a sharp increase in LacCer levels was observed within 2-5 minutes following stimulation with LPS/IFN [symbol] . Upon LPS/IFN [symbol] stimulation, LacCer levels increased ˜1.5 fold of those observed in unstimulated cells. Inhibition of lactosylceramide synthase (GalT-2, enzyme responsible for LacCer biosynthesis) by D-PDMP inhibited this increase in LacCer biosynthesis following LPS/IFN [symbol] stimulation. Additionally, when GalT-2 activity was assayed following LPS/IFN [symbol] stimulation, a rapid increase in enzyme activity with peak at 5 min following LPS/IFN [symbol] stimulation was observed (FIG. 3B). The role of GalT-2 and its product LacCer in iNOS gene regulation was further confirmed by using antisense DNA oligomers against rat GalT-2 mRNA and a sequence-scrambled oligomer as a control. As shown in FIG. 3C, antisense DNA oligomer reduced significantly the LPS/IFN [symbol] -mediated NO production and iNOS mRNA and protein levels (FIG. 3D). Furthermore, to investigate the possible role of LacCer and GalT-2 in iNOS gene regulation LacCer isolated and purified from these experiments was also investigated for its compositional and structural confirmation. Mass spectrometric analysis of LacCer from stimulated cells had agreement with 3 major fatty acids (18:0, 56.2%; 18:1, 26.4%; 16:0, 12.9%) in LacCer. LacCer consisting of 18:0 had the diagnostic present as m/z 889 (M, 1.1%), m/z 890 (M+H, 1.4%) and m/z 740 (M-[5×OH+2×CH₃OH], 41.6%). Similarly, 16:0 species of LacCer had the significant peaks present at m/z 861 (M+, 0.8%), 862 (M+H, 1.2%), m/z 860 (M−H, 1.1%) and m/z 711 (860-[5×OH+2×CH₃OH], 51.9%). The species of LacCer consisting of oleic acid (18:1) had a significant peak present at m/z 888 (M+H, 1.8%) and m/z 739 (888-[5×OH+2×CH₃OH], 100%). Two more important peaks present were at m/z 342 (M-sphingolipid backbone, 4.4%) and m/z 529 (M-Lac backbone-H₂O, 1.5%). LPS/IFN [symbol] stimulated cells had altered fatty acid profile as revealed by fatty acid methyl ester analysis by gas chromatography. All the saturated long chain fatty acids were found increased including 14:0 (167%), 16:0 (65.8%), 18:0 (7.3%) and 20:0 (5.7%) as compared with control cells. Taken together, the data from the GC and MS corroborated and confirmed the structure of purified compound as LacCer having major fatty acids as stearic, oleic and palmitic acid.

LacCer regulates LPS/IFN [symbol] -induced expression of iNOS In C6 glioma cells. To compare the role of LacCer in iNOS gene expression in C6 glioma cells, C6 cells were pretreated with increasing doses of D-PDMP (10, 25, 50 [symbol] M) for 0.5 hr before LPS/IFN [symbol] stimulation. NO production and iNOS protein and mRNA levels were measured after the incubation of C6 cells with LPS/IFN [symbol] Das described in the legend of FIG. 4. The LPS/IFN [symbol] induced NO production (FIG. 4A) and iNOS protein and mRNA levels (FIG. 4B) are inhibited in the presence of increasing doses of D-PDMP. However, upon the supplementation of exogenous LacCer, the inhibition is reversed evident by NO production (FIG. 4C) as well as iNOS mRNA and protein expression (FIG. 4D) whereas GluCer had no effect. These studies show that the LacCer mediated regulation of LPS-induced iNOS is similar in astrocytes and C6 glial cells.

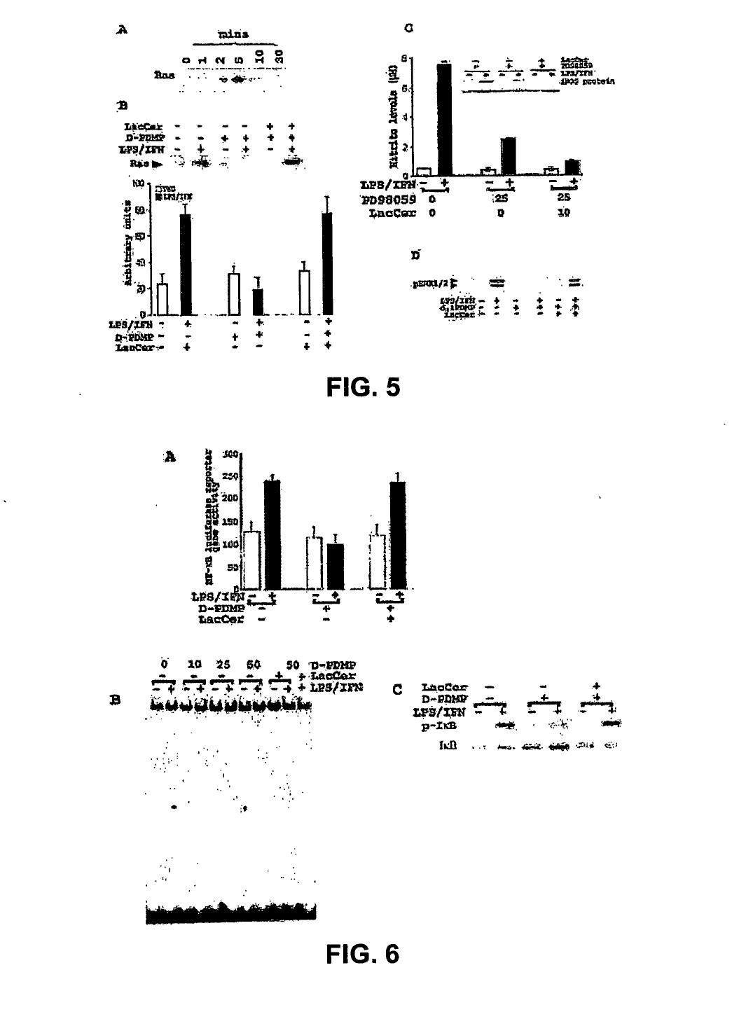

Activation of small GTPase Ras and ERK1/2 is involved in LacCer-mediated regulation of iNOS gene expression. To understand the role of LacCer in LPS/IFN [symbol] induced cellular signaling for induction of iNOS, the effect of LacCer on activation of small GTPases was investigated. The activation of small GTPase Ras is known to be critical for LPS/IFN [symbol] [symbol] induced iNOS gene expression (Pahan et al., 2000). Ras activation: was investigated by the use of GST-conjugated Raf-1 RBD (Ras binding domain). Upon LPS/IFN [symbol] [symbol] stimulation rapid activation of Ras was observed (FIG. 5A). The maximal LPS/IFN [symbol] mediated activation of Ras (observed within 2-5 mins following stimulation) was reduced by pretreatment with D-PDMP and this was fully reversed by exogenous supplementation of LacCer (FIG. 5B). These studies indicate that LacCer plays a role in the activation of Ras by LPS/IFN [symbol] resulting in the induction of iNOS gene expression. Furthermore, activation of extracellular regulated kinases 1&2 (which are downstream targets of Ras) was also observed upon LPS/IFN [symbol] stimulation. Pretreatment with D-PDMP inhibited the LPS/IFN [symbol] [symbol] induced phosphorylation of ERK 1/2 which was reversed in the presence of exogenous LacCer (FIG. 5D). Additionally, inhibition of a kinase responsible for ERK phosphorylation and activation, MEK1/2 by PD98059 resulted in inhibition of NO production and iNOS expression proving the involvement of ERK pathway in iNOS gene expression (FIG. 5C). Supplementation of exogenous LacCer had no effect on PD98059 inhibition of iNOS gene expression thus placing LacCer upstream as a second messenger molecule mediating regulation of LPS/IFN [symbol] induced iNOS gene expression through the Ras/MEK/ERK.

The role of NF- [symbol] B in LacCer mediated regulation of iNOS gene expression. To further understand the mechanism of LacCer-mediated iNOS gene regulation, the involvement of I [symbol] B/NF- [symbol] B pathway, which is known to be necessary for the induction of iNOS (Nunokawa et al., 1996; Pahan et al., 1998; Taylor et al., 1998; Keinanen et al., 1999), was examined. To test this possibility the effect of D PDMP on luciferase activity was observed in [symbol] B-repeat luciferase transfected cells. LPS/IFN [symbol] induced luciferase activity was abolished by D-PDMP treatment and was effectively bypassed by exogenous LacCer (FIG. 6A). As shown in FIG. 6B, NF- [symbol] B DNA binding activity tested by electrophoresis mobility shift assay was inhibited by increasing doses of D-PDMP but was reversed in the presence of exogenous LacCer. Specificity of NF- [symbol] B probe binding was proven by using 50× cold probe, which out-competed labeled NF- [symbol] B binding activity. As I [symbol] B phosphorylation and degradation is required for NF- [symbol] B activation and translocation to the nucleus, phosphorylated I [symbol] B levels were also examined. Decreased phosphorylation of I [symbol] B was observed in the presence of D-PDMP. However, when LacCer was added, the levels of phosphorylated I [symbol] B were increased which correlated with increased NF- [symbol] B nuclear translocation and DNA binding activity (FIG. 6C). These studies delineated the mechanism of LacCer mediated transcriptional regulation of LPS/IFN [symbol] induced iNOS gene expression to be through the I [symbol] B/NF- [symbol] B pathway.

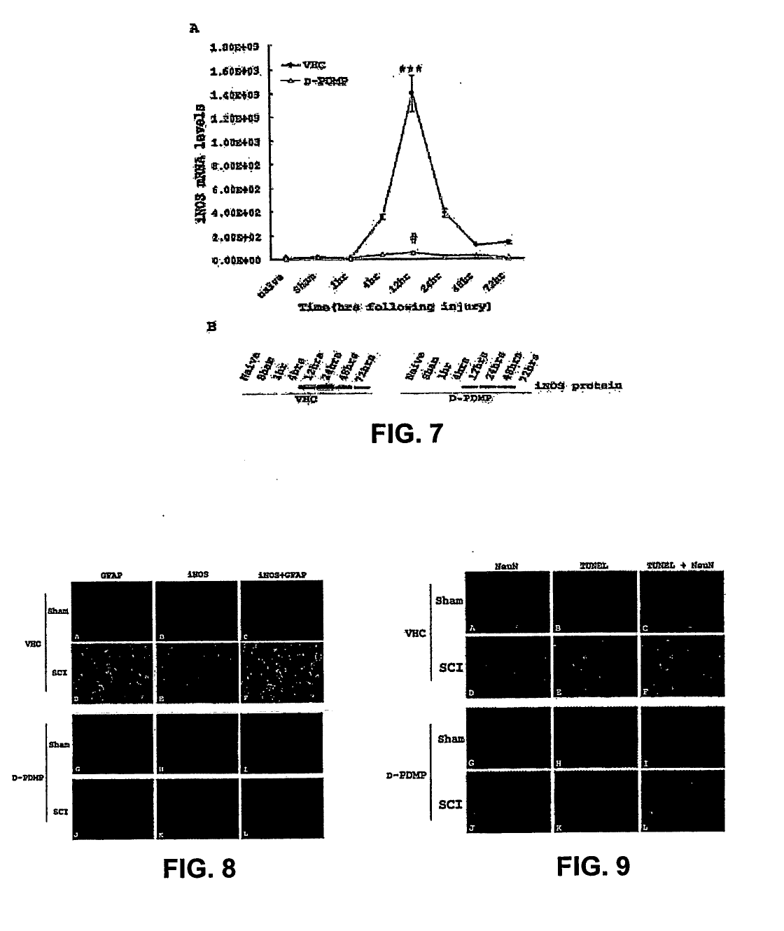

Efficacy of D-PDMP in controlling iNOS induction in spinal cord injury (SCI). To test the physiological relevance of previous observations and further investigate the role of LacCer in the induction of iNOS in neuro-inflammatory disease, the effect of D-PDMP in the rat spinal cord injury model was examined. Spinal cord injury in rats has been shown to result in rapid invasion of the lesion and the surrounding area by iNOS positive reactive astrocytes and macrophages (Wada et al., 1998a; Wada et al., 1998b). As shown in FIG. 7, a robust induction of iNOS mRNA measured by real time PCR (FIG. 7A) and protein expression (FIG. 7B) is observed 12 hrs followed SCI as compared to the Naïve or Sham operated animals. D-PDMP (20 mg/Kg) treatment within 10 minutes following SCI markedly suppresses this increase in iNOS expression. Double-immunofluorescence analysis of spinal cord sections from the site of injury of vehicle (VHC) and D-PDMP treated SCI rats showed a significant increase in GFAP (FIG. 8D) and iNOS (FIG. 8E) levels and their co-localization (FIG. 8F) 24 hrs following injury whereas D-PDMP treated SCI rats showed significantly reduced GFAP (FIG. 8J) as well as iNOS (FIG. 8K) expression demonstrating the efficacy of D-PDMP in vivo in controlling iNOS gene expression and the reactive transformation of astrocytes. These studies indicated involvement of glycosphingolipids in iNOS gene expression by reactive astrocytes at the site of lesion in an in vivo model of SCI and possible other neuro-inflammatory diseases and the efficacy of glycosphingolipid depletion by D-PDMP in inhibiting a iNOS induction, a major inflammatory event that worsens secondary damage.

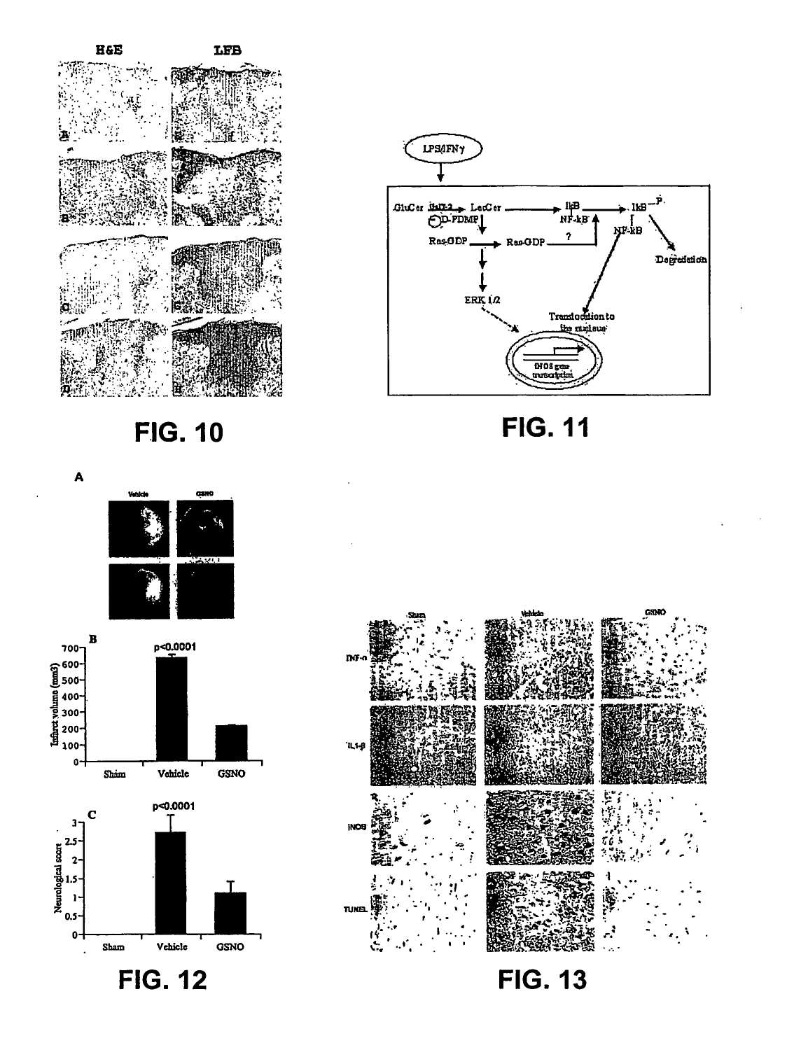

Attenuation of apoptosis and demyelination by inhibition of iNOS gene expression following SCI by D-PDMP. With respect to spinal cord impairment following trauma at the molecular levels, NO has been reported to be closely involved in the development of post-traumatic cavitation, neuronal death, axonal degeneration and myelin disruption. Significantly numerous TUNEL-positive cells were scattered in the lesion following SCI (FIG. 9E) some of which were also identified by double immunofluorescence staining as neurons using an anti-neuronal nuclei (NeuN) antiserum (FIGS. 9D and 9F). D-PDMP had a dual beneficial effect in the rat model of SCI. It could inhibit iNOS expression following SCI and furthermore as shown in FIGS. 9J-9L provided protection against apoptosis of neurons and other cells as well. This is of significant importance as no adverse effect of D-PDMP was observed on neuronal survival in sham operated animals (FIGS. 9G-9I) showing that the dose administered effectively inhibited iNOS without any obvious adverse effects which also translated in reduced SCI related pathology in terms of neuronal loss. Furthermore, as shown in FIG. 10, SCI induced white matter vacuolization and tissue necrosis observed by histological examination of injured rat spinal cord sections (FIG. 10B) was inhibited in tissue sections of SCI rats in which iNOS was inhibited by D-PDMP (FIG. 10D). The weight-drop injury is known to also result in myelin vacuolization resulting in locomotor dysfunction of the hindlimbs (Suzuki, et al., 2001). LFB staining of spinal cord sections for myelin from vehicle treated SCI rats showed profound demyelinization (FIG. 10F) which was also attenuated by D-PDMP mediated iNOS inhibition (FIG. 10H). Taken together these studies document that inhibition of inflammatory events such as iNOS expression in this study by D-PDMP has beneficial effects in animal model of SCI. They also underline the importance of glycosphingolipids in neuro-inflammatory disease.

### Example 3

### Discussion I

Nitric-oxide mediated pathophysiology is common to a number of neuroinflammatory diseases including stroke and spinal cord injury (SCI). As it is not completely known which factors induce and regulate iNOS gene expression in inflammatory disease, in this study the involvement of glycosphingolipids and demonstrated a novel pathway of iNOS gene regulation through LacCer mediated events involving Ras/ERK1/2 and the I [symbol] -B/NF- [symbol] B pathway in primary astrocytes has been investigated. These conclusions are based on the following findings. (1) LPS/IFN [symbol] [symbol] induced iNOS gene expression and LacCer production was inhibited by D-PDMP, a glycosphingolipid synthesis inhibitor. The addition of exogenous lactosylceramide, and not any other glycosphingolipid, reversed the inhibition of iNOS gene expression by D-PDMP. (2) LPS/IFN [symbol] stimulated GalT-2 activity within 5 minutes and rapidly increased the levels of intracellular lactosylceramide. Furthermore, knockdown of GalT-2 using antisense oligonucleotides resulted in decreased NO production and iNOS expression. (3) The pathway involved in iNOS regulation by LacCer is triggered by the small GTPase-Ras since D-PDMP abolished LPS induced Ras activation. ERK1/2 kinases further mediated iNOS expression as MEK1/2 inhibitor PD98059 inhibited LPS/IFN [symbol] mediated iNOS gene expression. (4), LacCer mediated transcriptional regulation of iNOS is through the I [symbol] -B/NF- [symbol] B pathway. D-PDMP inhibited LPS/IFN [symbol] -mediated induction of reporter gene activity of BB repeated minimal promoter as well as NF- [symbol] B transactivation and I [symbol] B degradation. As illustrated in FIG. 11, the following model is proposed for the events associated with LacCer mediated regulation of LPS/IFN [symbol] induced iNOS gene regulation. LPS/IFN [symbol] stimulation activated LacCer synthase (GalT-2), and increased intracellular LacCer levels. This translates into activation of the small G-protein Ras and the downstream extracellular regulated kinases 1&2 (ERK1/2) both of which have been demonstrated earlier to mediate cytokine induced iNOS gene expression and NF- [symbol] B activation (Pahan et al., 1998; Marcus et al., 2003). Additionally, whether through Ras activation or events mediated by itself, LacCer is able to trigger the I [symbol] B-NF [symbol] B pathway. NF- [symbol] B is an established major transcription factor for iNOS gene expression (Pahan et al., 1998). Phosphorylation of I [symbol] B results in its degradation, thus allowing NF- [symbol] B, which was sequestered by I [symbol] [symbol] in the cytosol, to be translocated into the nucleus and initiate iNOS gene expression. The data presented in this study identify LacCer, a ceramide derivative, as a signaling molecule in iNOS gene expression.

Since the discovery of the sphingomyelin (SM) cycle, several inducers (1a,25-dihydroxyvitamin D3, radiation, antibody crosslining, TNF [symbol] , IFN [symbol] , IL-1 [symbol] , nerve growth factor and brefeldin A) have been shown to be coupled to sphingomyelin-ceramide signaling events (Hannun, 1994; Kolesnick et al., 1994; Kanety et al., 1995; Linardic et al, 1996). Several studies support a role for hydrolysis of SM as a stress activated signaling mechanism in which ceramide plays a role in growth suppression and apoptosis in various cell types including glial and neuronal cells (Brugg et al., 1996; Wiesner and Dawson, 1996). Impairment of mitochondrial function resulting in enhanced production of reactive oxygen species (ROS) and decrease in mitochondrial glutathione thus generating oxidative stress has been delineated as one of the major causes of ceramide-induced cytotoxicity/apoptosis. The induction of manganese superoxide dismutase (MnSOD) by the SM signal transduction pathway has been identified as one of the ways by which ceramide-induced oxidative stress is controlled in primary astrocytes (Pahan et al., 1999). Amongst the agents causing oxidative stress, NO production can suppress growth and is an important candidate to induce apoptosis/cytotoxicity of neurons and oligodendrocytes in neurodegenerative diseases. It has recently been demonstrated that ceramide generated as result of neutral sphingomyelinase activation potentiates the LPS- and cytokine-mediated induction of iNOS in astrocytes and C6 glioma cells (Pahan et al, 1998). Furthermore, ceramide-mediated iNOS gene expression is shown to be through the Ras/ERK/NF- [symbol] B pathway (Pahan et al., 1998). Although ceramide itself does not induce iNOS gene expression and production of NO, it markedly stimulates the cytokine-induced expression of iNOS and NO production suggesting that sphingomyelin-derived ceramide generation may be an important factor in cytokine-mediated cytotoxicity in neurons and oligodendrocytes in neuroinflammatory disorders. Moreover, inhibition of LPS- and ceramide-induced expression of iNOS by antioxidant inhibitors of NF- [symbol] B activation (e.g N-acetyl cysteine and purrolidine dithiocarbamate) in astrocytes suggests a role for cellular redox in the ceramide-LPS or proinflammatory cytokine induced activation of NF- [symbol] B and induction of iNOS (Singh et al., 1998). The maintenance of the thiol/oxidant balance by N-acetyl cysteine (NAC), which serves as a scavenger of ROS and a precursor of GSH (natural anti-oxidant), also blocks cytokine-mediated iNOS expression and cermaide production through SM hydrolysis, thus preventing primary astrocytes and oligodendrocytes cell death by ceramide and NO (Singh et al., 1998). Furthermore, the efficacy of NAC treatment to protect against injury in a rat model of focal cerebral ischemia by inhibiting the expression of pro-inflammatory cytokines such as TNF [symbol] , induction of iNOS and cell death is also proven (Sekhon et al., 2003).

Instead of viewing enzymes of sphingolipid metabolism as isolated signaling modules, these pathways are now accepted to be highly interconnected with the product of one enzyme serving as a substrate for the other. This is also true of ceramide generated through the SM cycle or de novo, as ceramide can be converted into other bioactive molecules such as sphingosine, sphingosine-1-phosphate or glycosphingolipids. The complexity of these bioactive sphingolipids is accentuated by growing evidence of the presence of ceramide and other derivatives such as LacCer and gangliosides in lipid-enriched microdomains within membranes. These microdomains, called ‘lipid rafts’, have a number of receptors and signaling molecules localized within or associated with them thus making them hotspots for signaling events (Hakomori and Handa, 2003). The metabolic interconnections of ceramide and other lipids mediators such as sphingosine, sphingosine-1-phosphate (S-1-P) and glycosphingolipids make predicting the specific actions of these intermediates and the enzymes regulating their levels rather complex. For example, while sphingosine has pro-apoptotic effects like ceramide depending on cell type (Spiegel and Merrill, 1996), its rapid conversion to S-1-P has proliferative properties antagonistic to those of sphingosine and ceramide (Spiegel and Milstien, 2000). Of the glycosphingolipids, glucosylceramide and lactosylceramide, respectively, have been shown to promote the drug resistance state (Liu et al., 1999) and to mediate oxidized LDL and TNF [symbol] effects on superoxide formation, the activation of MAP kinase and the induction of proliferation in aortic smooth muscle cells (Chatterjee, 1998). It was sought to decipher the complexity of cytokine-mediated iNOS gene expression and further dissect the role of sphingolipids in this process. These studies defined the critical role of glycosphingolipids in iNOS gene regulation besides their other known functions in other cell systems. GalT-2 activation and LacCer biosynthesis was found to be critical for LPS- and cytokine-induced expression of iNOS. Although LacCer has been shown to mediate inflammatory events in other cell systems and influence ROS generation, apoptosis and cell proliferation, the role of LacCer in iNOS gene expression has not been established so far.

The novel mechanism of LacCer mediated iNOS gene regulation presented in this study found physiological relevance in CNS trauma when D-PDMP inhibited iNOS expression in the spinal cord in the rat model of spinal cord injury. D-PDMP was also able to inhibit neuronal cell loss by apoptosis and alleviate demyelination. As with most neurodegenerative conditions including spinal cord injury, the amount of spared tissue after injury along with apoptosis blocking therapies have been found to be beneficial for behavioral outcome and recovery following injury (Blight, 1983; Young, 1993; Liu et al., 1997), identification of LacCer as a key mediator of cytokine-induced iNOS gene expression underlines a major target with therapeutic potential to block apoptosis and iNOS expression in neurological diseases.

The other reported biological functions of LacCer, which include mediation of cytokine effects in inflammatory events such as generation of reactive oxygen species (Yeh et al., 2001), neutrophil adherence to endothelial cells by adhesion molecule expression (Arai et al., 1998; Bhunia et al., 1998), cell proliferation (Bhunia et al., 1997) and neutrophil activation (Iwabuchi and Nagaoka, 2002), are common to those observed during neuroinflammation. Thus, the involvement of LacCer in these events likely involves regulated adhesion molecules expression that results in the breakdown of the blood brain barrier and infiltration of immune cells, such as neutrophils, which synthesize proinflammatory cytokines and activate the resident microglia and astrocytes leading to ROS generation, NO production, neuronal apoptosis, demyelination and gliosis, which has a profound negative effect in injury and subsequent functional recovery (Hays, 1998; Akiyama et al., 2000a; Akiyama et al., 2000b). As the beneficial effects of antioxidant therapy using NAC and immunomodulation of the immune response by statins and diseases such as ischemia and EAE, respectively, is already known (Stanislaus et al., 2001; Sekhon et al., 2003), establishing a role for LacCer in mediating neuroinflammatory events such as ROS generation, neutrophil activation and infiltration, in addition to the role in iNOS induction as reported in this study, permits LacCer level modulation to be a multi-pronged tool to curb neuroinflammation.

In conclusion, this study reports for the first time the role of lactosylceramide in induction of iNOS in inflammatory disease. These studies identify a new therapeutic target of glycosphingolipid modulation for amelioration of pathophysiology in neuroinflammatory disorders.

### Example 4

### Materials and Methods II

Reagents. An ApopTag® plus peroxidase in situ detection kit (S7101) was obtained from Intergen (CITY, N.Y.). Mouse monoclonal TNF-antibody (SC-7317) and rabbit polyclonal IL-1 antibody (SC-7884) were obtained from Santa Cruz Biotechnology, Inc. (Santa Cruz, Calif.). Rabbit polyclonal iNOS antibody (N32030-050) was obtained from Transduction Laboratories (San Diego, Calif.). GSNO was purchased from World Precision Instruments, Inc. (Sarasota, Fla.). DMEM (4.5 gm glucose/L), RPMI 1640 medium, fetal bovine serum and Hanks balanced salt solution were from Life Technologies (Grand Island, N.Y.). All other chemicals and reagents used were purchased from Sigma (St. Louis, Mo.) unless stated otherwise.

Animals. A total of 60 male Sprague-Dawley rats (Charles River Laboratories, Wilmington, Mass.) weighing 250-300 g were used in this study. All animals received humane care in compliance with the Medical University of South Carolina's guidance and the National Research Council's criteria for humane care as outlined in ‘Guide for the Care and Use of Laboratory Animals’. Body temperature was monitored by a rectal probe and maintained at 37±0.5° C. by a homeothermic blanket control unit (Harvard Apparatus, Holliston, Mass.). Cranial temperature and mean arterial blood pressure were measured by HSE Plugsys TAM-D and TCAM (Harvard Apparatus) respectively.

Experimental design and administration of drugs. All animal procedures were approved by the Medical University of South Carolina Animal Review Committee and were in accordance with the guidelines for animal use published by the National Institute of Health. The animals were divided into three groups: (i) control (sham-operated) group (Sham, n=20); (ii) ischemia (20 min) and reperfusion (24 h) group (Vehicle, n=20); (iii) GSNO treatment group (GSNO, n=20). In the treatment group, GSNO (1 mg/kg body weight) solution in saline (˜250 l) was slowly infused by femoral vein cannulation at the time of reperfusion. The rats in the ischemia (vehicle) and control (sham) groups were administered the same volume of normal saline instead of GSNO.

Focal cerebral ischemia model. Rats were fasted overnight but allowed free access to water before the experiments. Rats were anesthetized with an intraperitoneal injection of xylazine (10 mg/kg body weight) and an intramuscular injection of ketamine hydrochloride (100 mg/kg). A rectal temperature probe was introduced, and a heating pad maintained the body temperature at 37±0.5° C. Right MCA was occluded as described in the inventor's earlier publication (Sekhon et al. 2003). The surgical procedure was completed in 15 min and did not involve significant blood loss. At the end of ischemic period, the nylon monofilament was withdrawn, the common carotid artery clamps were removed, and reperfusion through the common carotid artery was confined microscopically. The animals were then allowed to recover from anesthesia on a warming pad. The animals were sacrificed after a specified period of reperfusion time. Brains of the rat were divided in two parts, identified as the ischemic hemisphere (ipsilateral) and the ischemia-unaffected (contralateral) regions and then immediately either used for analysis or frozen in liquid nitrogen and stored at −70° C. for analysis later.

Measurement of physiological variables. The physiological variables were measured before, during MCA occlusion, at reperfusion and 30 min after reperfusion. The rectal temperature was monitored and maintained at about 37 to 37.6° C. Regional cerebral blood flow (rCBF) was examined using laser Doppler flowmeter (Perimed Sweden and Oxford Optronix Ltd., Oxford, UK) in experimental animals and sham controls.

Evaluation of ischemic infarct and neurological score. A 2,3,5,-triphenyltetrazolium chloride (TTC) staining technique was used for the evaluation of ischemic infarct followed by image acquisition by computer. Briefly, after an overdose of pentobarbital, the rats were killed by decapitation after 24 h of reperfusion. The brains were quickly removed and placed in ice-cold saline for 5 min. Six serial sections from each brain were cut at 2-mm intervals from the frontal pole by Brain Matrix (Brain Tree Scientific). The sections were incubated in 2% TTC (Sigma, Mo.) and dissolved in saline for 15 min at 37° C. The stained brain sections were stored in 10% formalin and refrigerated at 4° C. for further processing and storage. Coronal sections (2 mm) were placed on a flat bed color scanner (HP scan jet 5400 C) connected to a computer. The infarct area, outlined in white, was acquired by image-analysis software (Photoshop 4.0 Adobe System) and measured by NIH image software. Neurological evaluation was performed by an observer blinded to the identity of the group. Neurological deficits were assessed at 30 min, 24 h, and 72 h after reperfusion (before sacrifice) and scored as follows: 0, no observable neurological deficit (normal); 1, failure to extend left forepaw on lifting the whole body by tail (mild); 2, circling to the contralateral side (moderate); 3, leaning to the contralateral side at rest or no spontaneous motor activity (severe). The animals not showing neurological deficits at the above time-points were excluded from the study as described in the inventor's earlier publication (Sekhon et al. 2003). In a subset of animals treated with saline or GSNO, CBF was measured before, during MCAO, and 3 hr after start of reperfusion. Each animal, under anesthesia, was placed in a stereotaxic apparatus and a needle probe was placed at bregma with the following coordinates (anterioposterior, −1.0 mm; lateral, −4.0 mm).

Measurement of Caspase-3 Activity in rat brain. Caspase-3 enzyme activity assay was carried out as described earlier (Haq et al. 2003). Briefly, the reaction mixture contained 50 g of cytosolic protein prepared from rat brain homogenates and 500 M Ac-DEVD-AMC (caspase-3 substrate II, fluorogenic; Caibiochem Cat# 235425) in 900 l of buffer B (100 mM HEPES, pH 7.4; 20% glycerol; and 2 mM dithiothreitol). The enzyme reaction was initiated by adding the substrate to the tissue extract and incubated at 37° C. The caspase-3 like activity was measured using a spectrofluorometer at an excitation wavelength of 380 nm and an emission wavelength of 460 nm for detecting the shift in fluorescence upon cleavage of AMC fluoropore.

Cell culture. Primary rat astrocytes were prepared from 1-3 day old postnatal Sprague-Dawley rat pups and maintained in DMEM (4.5 gm glucose/L) with 10% o fetal bovine serum (FBS) and antibiotics. Based on GFAP (glial fibrillary acidic protein) positive immunostaining, astrocytes were determined to be more than 95% pure. BV2 cell is a microglia cell line derived from murine primary microglia provided by Dr. Michael McKinney and maintained in DMEM (4.5 gm glucose/L) supplemented with 10% FBS and antibiotics. Cytotoxic effects of treatments were determined by measuring the metabolic activity of cells with 3-(4,5-dimethyl thiazol-2-yl)-2,5-diphenyl tetrazolium bromide (MTT) and LDH release assay (Roche).

Western blot analysis. Fresh or frozen brain tissue or cultured cells were used for the Western blot analysis. Tissues (brain) were homogenized in an ice-cold buffer containing 20 mM Tris. pH 7.4; 150 mM NaCl; 1 mM EDTA; 1 mM EGTA; 1% Triton; 2.5 mM Na pyrophosphate; 1 mM vanadate; 1 g leupeptin. The homogenates (160 g protein each) were treated with cold acetone, vortexed, and stored at −70 C for 4 h. The samples were centrifuged at 12,000×g for 10 min to precipitate the protein. The dry pellets were then boiled for 5 min in loading buffer. Equal amounts (40 g of protein per lane) of protein was subjected to SDS-PAGE analysis and transferred to nitrocellulose (Amersham). Samples from the cultured cells for immunoblot were prepared as described earlier (Giri et al. 2002). Briefly, the cells were harvested and then lysed in ice-cold lysis-buffer (50 mM Tris-HCl, pH 7.4, containing 50 mM NaCl, 1 mM EDTA, 0.5 mM EGTA, 10% glycerol, and protease inhibitor cocktail). The samples were centrifuged at SEE P. 12 for 10 min. Supernatant (50 g protein/lane) was analyzed by SDS-PAGE and blotted to nitrocellulose (Amersham). Blots were blocked for 1 h in 5% nonfat dry milk-TBS-0.1% Tween 20, washed, and then incubated overnight with iNOS antibody (1:1000) in 5% BSA-TBS-0.1% Tween 20 at 4° C. and then washed. This was followed by incubation for 1 h with rabbit secondary peroxidase conjugated antibody (1:5,000, Sigma). Immunoreactivity was detected using the enhanced chemiluminescence detection method according to the manufacturer's instructions (Amersham Pharmacia Biotech) and subsequent exposure of the membrane to X-ray film. Densitometric analysis was performed on a Bio-Rad densitometer (Model GS-670, imaging densitometer). Protein concentration was determined using a commercially available protein assay dye (Bradford reagent) from Bio-Rad.

Transcriptional assays. Primary astrocytes or microglial cell line (BV2) were transiently transfected with NF-B—or iNOS-luciferase reporter gene (1.5 g/well) with -galactosidase by lipofectamine-2000 (Invitrogen) for astrocytes and lipofectamine-Plus (Invitrogen) for BV2 cells, according to the manufacturer's instructions in 12-well plates as described (Giri et al. communicated). For co-transfection studies, cells were transfected with reporter in the presence or absence of HA-IKKa or HA-IKK or p65 or p50 expression vector. Total DNA (3 g/well) was kept constant and pcDNA3 was used to normalize all groups to equal amounts of DNA. Luciferase activity was determined using a luciferase kit (Promega).

Apoptosis by TUNEL Assay. Apoptosis was detected by TUNEL (TdT-mediated dUTP nick end labeling) assay. Briefly, sections were deparaffinized with xylene and rehydrated through three changes of graded alcohol and incubated in phosphate buffer saline (PBS) for 15 min at room temperature and then in 20 g/ml proteinase K for 15 min at room temperature directly on the side. The ApopTag® plus peroxidase kit (Intergen Company) was used for detection of apoptosis. Endogenous peroxidase activity in the brain sections was blocked by incubation with 3% H₂0₂ in PBS for 5 minutes followed by incubation for 10 seconds with equilibration buffer.

The sections were incubated for 60 min at 37° C. with terminal deoxynucleotidyl transferase (TdT) in reaction buffer. The reaction was terminated by incubation with stop/wash buffer at room temperature. Sections were then incubated with peroxidase conjugated anti-digoxigenin antibody (affinity purified sheep polyclonal) for 30 min at room temperature and the reaction was developed with diaminobenzidine (DAB) substrate for 4 min at room temperature. Sections were counter-stained with methyl green for 30 sec and mounted in Permount (Fisher Scientific, Fair Lawn, N.J.). Double labeling was used to identify TUNEL positive cells by developing the peroxidase reaction with the fluorescent substrate Cyanine 3 Tyramide (peroxidase substrate supplied with TSA-Direct kit, NEN Life Sciences, Boston, Mass.) in place of DAB, followed by incubation with neuronal specific enolase antibody (rabbit polyclonal 1:100, Chemicon, Temecula, Calif.), and visualized with anti-rabbit FITC (1:100, Vector Labs, CA).

Immunohistochemistry. Cytokine (TNF- & IL-1) and iNOS expression was detected by immunohistochemical analysis using specific antibodies. Paraffin embedded sections from the formalin fixed brain tissues were stained for TNF-, IL-1 and NOS. In brief, the brain tissue sections were deparaffinized, sequentially rehydrated in graded alcohol, and then immersed in phosphate-buffered saline (PBS, pH 7.4). Slides were then microwaved for 2 min in antigen unmasking solution (Vector Labs, CA), cooled and washed 3 times for 2 min in PBS. Sections were immersed for 25 min in 3% hydrogen peroxide in distilled water to eliminate endogenous peroxidase activity, then blocked in immuxohistochemical grade 1% bovine serum albumin in PBS for 1 h and diluted goat serum for 30 min to reduce non-specific staining. Sections were incubated overnight with primary mouse monoclonal TNF-antibody (1:50, Bio Source), IL-1 antibody (1:25, Santa Cruz Biotechnology, CA) and rabbit polyclonal iNOS antibody (1:10, Transduction Labs, CA) diluted in blocking buffer and then rinsed 3 times for 6 min in PBS containing 0.1% Tween-20. iNOS and IL-1 was detected with anti-rabbit biotinylated antibody and TNF- with anti-mouse followed by an avidin-biotin HRP complex (Vectastain ABC-Elite Kit, Vector Labs, CA) with diaminobenzidine as substrate. The slides were then dehydrated through a graded series of alcohol and mounted in Permount and coverslipped. All the sections were analyzed using an Olympus microscope and images were captured using a digital video camera (Optronics) controlled by Adobe Photoshop (Adobe Systems, CA).

For immunofluorescent double-labeling, sections were incubated first with anti-iNOS (1:10) followed by macrophage marker ED1 (1:100, mouse monoclonal, ARU0151 from Biosource, Camarillo, Calif.) or astrocyte marker GFAP (1:100, mouse monoclonal, clone 6F-O1 cat # YM-3012 from Accurate, Westbury, N.Y.). Anti-iNOS was visualized using Texas Red conjugated anti-rabbit IgG (1:100, Vector Labs, CA) and ED 1 or GFAP using FITC conjugated anti-mouse IgG (1:100, Vector Labs, CA). Rabbit or mouse polyclonal IgG was used as control primary antibodies. Sections were also incubated with FITC or Texas Red conjugated IgG without the primary antibody as negative control. After washing, slides were air dried and mounted with aqueous mounting media (Vector Labs). Slides were examined for immunofluorescence using an Olympus microscope equipped for epifluorescence with dual wavelength filter and Adobe Photoshop software. Individual color channels (red or green) were separated with Adobe Photoshop software.

Statistical analysis. All values are expressed as mean ±SD. Comparisons among means of groups were made with a two-tailed Student's t test for unpaired variables. Differences among groups were considered significant when p<0.05.

### Example 5

### Results II

Effect of GSNO on reduction of infarction and on recovery of neurological score. TTC-stained representative sections (numbers 3 and 4 of a total of 6 sections arranged from cranial to caudal regions) from saline-treated ischemic brains (vehicle) and GSNO-treated (GSNO) ischemic brains are presented in FIG. 12A. Treatment with GSNO, compared with vehicle, reduced the infarct as measured by the disappearance of significant areas of the brain's white region. The infarct volume (FIG. 12B), which was based on all 6 slices, was found to decrease significantly. There was a significant difference in the neurological scores between ischemic and GSNO-treated animals (FIG. 12C). Twenty min of MCA occlusion and 24 hr of reperfusion led to a neurological score of 2.70±0.48. GSNO-treated animals had an average neurological score of 1.10±0.32. The selection of dose of GSNO (1 mg/kg body weight) is based on maximal brain protection (infarct volume). This dose had no effect on resting blood pressure, intracranial pressure, and other physiological parameters. However, administration of GSNO after the onset of ischemia was associated with significantly increased cerebral blood flow (CBF) 128.0% vs 11.2% (average value from 2 animals in each group) 3 h after is ischemia. The survival of animals were also monitored up to 7 days after ischemia both in GSNO-treated and untreated groups. While all the treated animals (n=7) survived up to 7 days and remained healthy and free from neurological deficits, the untreated animals (n=7) died within 3 days after ischemia.

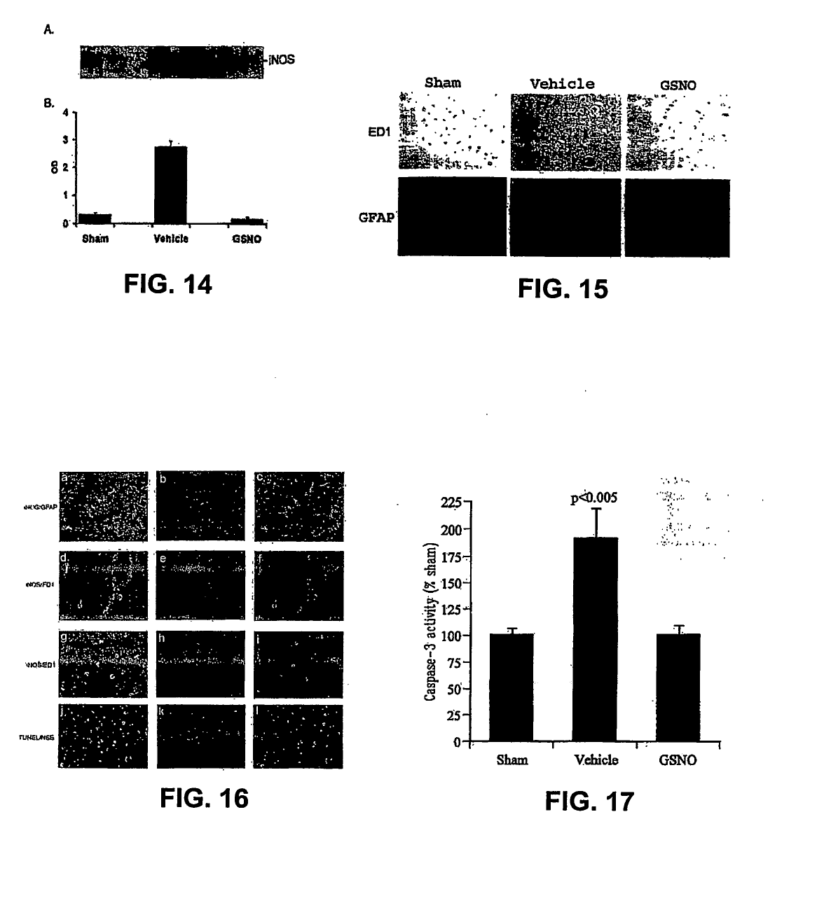

Effect of GSNO on the induction of cytokines and iNOS. TNF-α-mediated induction of iNOS after brain ischemia and reperfusion is related to the production of substantial amounts of NO. NO then reacts with 02⁻ to form ONOO⁻, a potent oxidant, which is directly implicated in cell death and indirectly causative via generation of hydroxyl radicals. TNF-α (FIGS. 13A-C) and IL-1β (FIGS. 13D-F) were found to be highly expressed after 24 h of reperfusion in the ipsilateral hemisphere (mainly cortex) of the ischemic brain and this expression was significantly reduced in GSNO-treated animals. The expression of iNOS (FIGS. 13G-I) detected by immunostaining after 24 h reperfusion followed a similar pattern as TNF-α and IL-1β, and treatment with GSNO reduced drastically the number of iNOS positive cells. The sham-operated group had no iNOS positive cells present in any examined region of the brain. The presence of expression of iNOS in the ipsilateral hemisphere and its absence in the GSNO-treated ipsilateral hemisphere of ischemic brains were also supported by Western blot analysis as shown in FIGS. 14A and 14B. The expression of iNOS was found mainly in macrophage/microglia as the staining for iNOS merged with the expression of ED-1 (FIG. 16D-16I). Macrophages/microglia expressing iNOS were present in the cortex region (FIG. 16G-16I) as well as in vessels (FIG. 16D-16F). The expression of iNOS also merged with GFAP, a marker for activated astrocytes (FIG. 16A-16C), thereby indicating the participation of activated astrocytes in iNOS-induced nitrosative stress.

Effect of GSNO on the expression of ED-1 and GFAP. Experimental evidence suggests that ischemic damage and the progression of the infarction proceed at a slow pace until the involvement of inflammatory mediators. Inflammatory mediators, secreted by either infiltrating blood bome cells or by activated glial cells, enhance oxidative and nitrosative stresses and induce apoptotic cell death. EDI was used to detect cells of monocyte origin, including activated microglia. There was significant specific staining for EDI (FIG. 15B) in the penumbra region of ipsilateral hemisphere (vehicle). Treatment with GSNO reduced the number of EDI positive cells (FIG. 15C). The sham group had no staining for EDI (FIG. 15A). In ischemia, the infarct is surrounded by a large number of hypertrophic astrocytes expressing high levels of glial fibrillary acidic protein (GFAP, an astrocyte-specific cytoskeletal protein). GFAP-positive astrocytes were found increased significantly in ischemic cortex region as shown in FIG. 15E. Treatment with GSNO decreased the number of activated astrocytes (FIG. 15F).

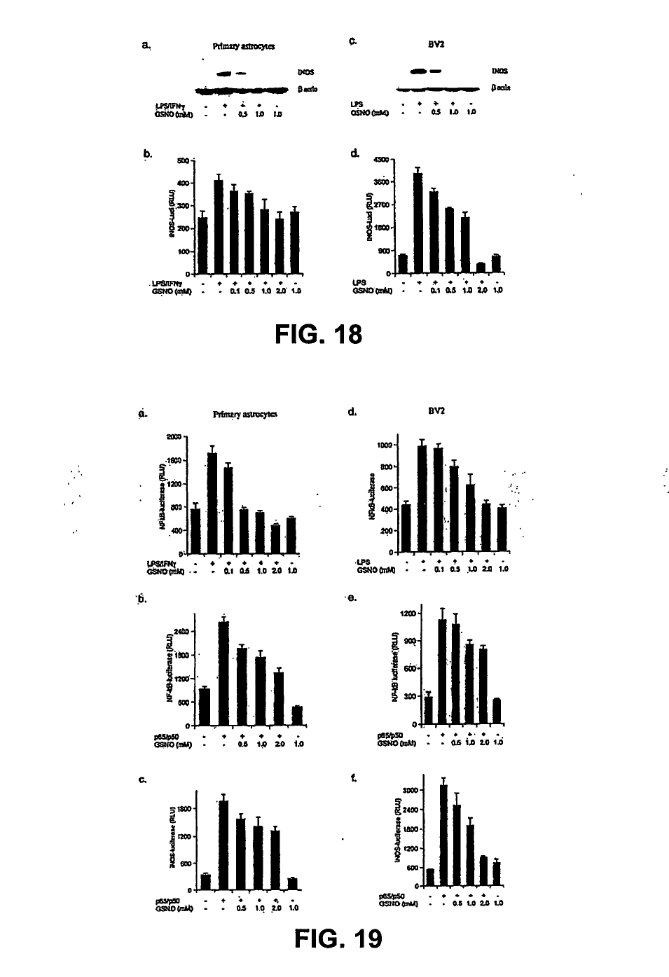

Effect of GSNO on apoptotic cell death and caspase-3 activity. DNA fragmentation as an indicator of apoptosis was determined by transferase-mediated d-UTP-labeled nick end labeling (TUNEL) assay. DNA fragmentation in ipsilateral hemisphere especially around the border of infarct was increased significantly (FIGS. 13J-13L). GSNO treatment resulted in a decreased number of apoptotic cells. Control brain as well as the contralateral hemisphere of the ischemic brain did not show TUNEL positive cells. The fact that the TUNEL positive cells were mainly neurons was confirmed by merging the TUNEL staining with the neuron specific marker NSE (FIGS. 16J-16L). Furthermore, the caspase-3 activity was found to be significantly increased in the ipsilateral hemisphere of ischemic brain as compared to sham operated, and this activity returned to a basal level in GSNO treated brain (FIG. 17).

Effect of GSNO on cytokine-induced expression of iNOS in rat primary astrocytes and microglia (BV2). The production of NO in response to cytokines has been shown to be important in the pathobiology of cerebral ischemia. In order to investigate the mechanism involved in inhibition of iNOS by the treatment with GSNO, rat primary astrocytes and microglia were used for in vitro studies, as these cells are involved in the propagation of inflammation in the brain after ischemic insult. The cells were pretreated with different concentrations (0.1 to 2 mM) of GSNO and then treated with either LPS (1 μg LPS/ml) in case of BV2 or LPS (1 μg LPS/ml)+IFN-γ (50 U IFN-γ/ml) for astrocytes. After 24 h, the cells were analyzed for iNOS protein by Western blot. Pretreatment with GSNO inhibited the expression of iNOS both in astrocytes (FIG. 18A) and BV2 (FIG. 18C) in a dose-dependent manner. GSNO (1 mM) alone had no effect on iNOS expression. The inhibitory effect of GSNO on cytokine-induced iNOS expression was further confirmed by iNOS luciferase activity assay both in astrocytes (FIG. 18B) and BV2 (FIG. 18D) cells.

Effect of GSNO on cytokine-induced NF- [symbol] B luciferase activity in rat primary astrocytes and BV2. To understand the mechanism of GSNO-mediated down regulation of iNOS expression, the effect of GSNO on LPS/IFN-γ or LPS-mediated NF- [symbol] B activation in astrocytes and BV2 cells respectively was investigated. NF- [symbol] B consists of a p65/p50 heterodimer and is retained in cytoplasm by its association with IγB in non-stimulated cells. Cytosolic NF- [symbol] B/IγB complex dissociates and free NF- [symbol] B translocates to the nucleus and regulates the transcription of NF- [symbol] B responsive genes including iNOS after stimulation of cells. Phosphorylation of IγB by the upstream kinase IKK is essential for the dissociation of IγB from NF- [symbol] B. Activation and translocation of NF- [symbol] B to nucleus (Hallenbeck 2002; Han et al. 2003) have been shown to be critical for the expression of iNOS and pro-inflammatory cytokines (TNFα, IL-1β and IL-6β). The inhibitory effect of GSNO on NF- [symbol] B activity was further analyzed in cells transfected with the NF- [symbol] B luciferase vector, by monitoring the reporter activity in response to LPS/IFN-γ or LPS challenge. Treatment with different concentrations of GSNO (0.1 to 2 MM) decreased the LPS/IFN-γ or LPS-dependent activation of NF- [symbol] B luciferase reporter activity in rat primary astrocytes and BV2 cells, respectively (FIGS. 19A, 19D). To investigate the direct effect of GSNO on NF- [symbol] B luciferase activity, the expression vectors of p65 and p50 subunits of NF- [symbol] B along with NF- [symbol] B luciferase reporter were cotransfected in primary astrocytes and BV2 cells. After 48 h of transfection, cells were treated with different concentrations of GSNO followed by LPS or LPS/IFNγ for 5 h. GSNO treatment inhibited p65/p50 mediated luciferase activity in primary astrocytes as well as in BV2 cell line (FIGS. 19B, 19E) in a dose-dependent manner. GSNO also attenuated p65/p50 mediated iNOS-luciferase in these cells (FIG. 19C, 19F) further suggesting that GSNO also mediated its effect directly on NF- [symbol] B subunits and modified their ability to bind to DNA for the transcription of pro-inflammatory genes participating in injury. The effect of GSNO on IKK or IKK-mediated iNOS- and NF- [symbol] B luciferase activity were also examined in astrocytes and BV2 cells. Treatment of astrocytes and BV2 cells with GSNO inhibited IKK and IKK mediated iNOS-luciferase and NF- [symbol] B luciferase activity in dose dependent manner (data not shown) suggesting GSNO might be affecting the up stream of IKKs to inhibit NF- [symbol] B pathway.

### Example 6

### Discussion II

Treatment with GSNO inhibited the expression of TNF-α, IL-1 and iNOS and reduced apoptosic neuronal cell death in the ipsilateral hemisphere of the brain in a rat model of experimental stroke. This in turn resulted in protective effects both in terms of reduction of infarction (FIGS. 12A-12B) and improvement in neurological score (FIG. 12C). The conclusion of neuroprotection by GSNO is based on the observations that GSNO treatment inhibited the activation of astrocytes and microglia/macrophage and reduced inflammation. The treatment also inhibited the induction of iNOS expression and reduced apoptosis of neurons. Furthermore, the treatment in vitro with GSNO inhibited cytokine-induced iNOS induction and cytokine-mediated NF- [symbol] B luciferase activity both in rat primary astrocytes and BV2 cells (FIG. 18-19).

Evidence now suggests that stroke has inflammatory components and may be amenable to treatment by anti-inflammatory agents (Barone and Feuerstein, 1999). TNF-(Hallenbeck, 2002) and IL-1β (Rothwell, 2003) are considered to be responsible for the accumulation of inflammatory cells in the injured brain and play a complex but significant role in stroke pathobiology. As a matter of fact, inflammation has been identified a major mechanism of injury during reperfusion in a model of focal cerebral ischemia (Kato et al. 1996; St oil et al. 1998). The inventors have already shown that inhibition of cytokines and iNOS by the antioxidant, N-acetylcysteine, is neuroprotective (Sekhon et al., 2003; Sekhon 2002). This has been further proved by other studies, which show the compounds that inhibit iNOS are protective in focal cerebral ischemia even when administered after the insult (Ding-Zhou et al., 2002; Zhu et al., 2002). Mice deficient in the iNOS gene (Zhao et al., 2000) show reduction in infarct volumes compared with respective controls. Aminoguanidine, a selective iNOS inhibitor, suppresses iNOS activity in mice with brain ischemia to levels equivalent to those seen in iNOS knockout mice, confirming that this enzyme is involved in ischemic injury (Sugimoto and Iadecola 2002). L-arginine has been shown to increase ischemic injury in wild-type mice but not in iNOS-deficient mice suggesting that L-arginine used by iNOS to produce NO is toxic in ischemic injury (Zhao et al., 2003). The expression of iNOS has been shown in many cell types in brain after ischemia/reperfusion (Dimagl et al., 1999). Evidence has been extended for the presence of expression of iNOS in human brain after ischemic infarction (Forster et al., 1999). The expression of iNOS in macrophage/microglia (EDI) and in activated astrocytes (GFAP) was identified by immunohistochemistry in ipsilateral hemisphere of ischemic animals (FIGS. 16A, 16G). EDI positive cells showing iNOS expression were also present in vessels (FIG. 16D) as has been observed by others. Inhibition of inflammation by anti-inflammatory/neuroprotective agents including NO donors and iNOS inhibitors has been an attractive hypothesis regarding cerebral ischemia. Both sodium nitroprusside (SNP) and spermine/NO have been shown to protect brain from injury in a rat model of focal cerebral ischemia (Salom et al., 2000). SNP is a salt that protects brain probably by the effect of NO through guanylyl cyclase and cGMP (Zhang et al., 1994a). The protection offered by SIN-1, a donor of NO and O′ has been disputed and argued that its effect is dependent on ischemic brain acidosis (Coert et al., 2002). A neuroprotective agent, GSNO, was used in this study. It is recognized as NO-donor, antioxidant and S-nitrosylating agent (Chiueh 2002). The results suggest that in focal cerebral ischemia, treatment by GSNO not only protected the brain from ischemic injury, but also ameliorated the inflammation by inhibition of expressions of TNFα, IL-1β and iNOS in brain resident activated cells and infiltrated blood borne cells.

GSNO is a stable metabolite of glutathione and NO, and is formed during the oxygen-dependent oxidation of NO in the presence of GSH (Hogg, 2000; Jourd'heuil et al., 2003; Schrammel et al., 2003). It is present in high concentrations in rat brain and releases NO slowly and regulates nitrosylation/denitrosylation under physiological conditions (Kiuge et al., 1997). Its decay is considered complex and is dependent on several factors (Ford et al., 2002; Zeng et al., 2001). GSNO has been shown to be a potent inhibitor of platelet aggregation (Langford et al., 1994) and reduces embolization in human (Molloy et al., 1998). It is highly effective in rapidly reducing the frequency of embolic signals in a set group of patients (Kaposzta et al., 2002a; Kaposzta et al., 2002b). It can reverse acute vasocontriction and prevent ischemic brain injury after subarachnoid hemorrhage (Sehba et al., 1999). Systemic administration of GSNO during balloon injury and intracoronary radiation resulted in reduction of thrombosis rate in swine (Vodovotz et al., 2000). GSNO inhibits clotting factor XIII to stop platelet aggregation (Catani et al., 1998). In addition, GSNO is a more potent antioxidant than GSH against ONOO⁻ and HO. There is substantial evidence that S-nitrosylation/transnitrosylation serves as an important mediator of NO-related bioactivity, both in NOS containing cells and in other cells via intercellular signaling. Recently, it has been documented that GSNO, like NO, 0₂ and H₂0₂, is a signaling molecule serving between endothelial or astrocytes and neurons (Chiueh and Rauhala 1999). Rauhala et al. have also documented neuroprotection by GSNO of brain dopamine neurons from oxidative stress (Rauhala et al., 1998). In the present study, the administration of GSNO even after the onset of occlusion protected not only the brain from infarction but also improved neurological score. The protection provided by GSNO in this acute stroke model may be explained in terms of multimodal involvement of GSNO itself and its metabolites including NO and GSH (Chiueh and Rauhala 1999). GSNO has the capacity to modulate blood vessel tone (Rodriguez et al., 2003). NO released from GSNO may have preserved, at least in part, the endothelial function through binding with guanylyl cyclase, hence increasing cGMP level and cerebral blood flow. Cerebral blood flow was monitored up to 3 h after ischemia and found a significant increase in cerebral blood flow (data not shown). A new dimension to NO signaling is the direct cGMP-independent action by RSNO in general and by GSNO in particular through S-nitrosylation/denitrosation and transnitrosation (Foster et al. 2003). However, the main focus was to investigate the anti-inflammatory effect of GSNO in acute stroke. With this aim, brain cells including rat primary astrocytes and BV2 cell line (microglia lineage) were treated. GSNO inhibited the iNOS expression dose dependently as is evident in FIG. 18. Inhibition was further confirmed by iNOS-luciferase activity (FIG. 18C-18D). Further, the inventor investigated the mechanism of iNOS inhibition by GSNO and found this to be mediated by NF- [symbol] B as shown by inhibition of NF- [symbol] B luciferase activity by GSNO in a dose dependent manner. GSNO inhibited cytokine-induced expression of iNOS gene, perhaps through a mechanism involving NF- [symbol] B inactivation in rat primary astrocytes and BV2 cell. This effect may decrease the damage to cells by NF- [symbol] B responsive inflammatory genes. Inhibition of NF- [symbol] B by S-nitrosylation of thiol group of p50 using S-nitrosocysteine has been documented in murine macrophages and human respiratory cells (Marshall and Stamler, 2001). At least two targets of NO inhibition of NF- [symbol] B activating pathway exist, one in cytoplasm (possibly the IKK complex) and the other in nucleus (p50-p65) depending on cell type (Marshall and Stamler 2002). It was hypothesize that, in this case, GSNO nitrosylated p50-p65. Nitrosylation of p50 inhibited the binding of p50-p65 to DNA in the promotor region of iNOS as shown in FIG. 7. This may be the first report indicating that GSNO inhibited iNOS expression in rat primary astrocytes involving the NF- [symbol] B pathway. Although, inhibition of cytokine-mediated activation of NF- [symbol] B and chemokines by GSNO in keratinocytes has been reported previously (Giustizieri, 2002). Cell death in focal cerebral ischemia/reperfusion occurs both by necrosis as well as by apoptosis (Kametsu et al., 2003; Yao et al., 2001). The ratio of necrosis versus apoptosis is dependent on several major factors. Time of ischemic events, type of occlusion, age and predisposition to risk factors are major determinants of stroke severity and apoptotic cell death (Love, 2003). The regulatory role of NO as apoptotic or anti-apoptotic is complex and involves different mechanism (Chung et al., 2001; Kim et al., 2001; Kim et al., 1999). Intervention by anti-apoptotic drugs to rescue the cells from apoptosis is one of the primary achievable goals in clinic (Waldmeier 2003). Because oxidative DNA damage precedes DNA fragmentation after experimental stroke in rat brain (Cui et al., 2000), antioxidant and neuroprotective agents may be used to protect DNA from oxidation and fragmentation. This objective led to the use of a drug that may have anti-apoptotic along with anti-ischemic properties. A significant number of apoptotic neurons were observed (FIGS. 16J-16L) in penumbra as shown by TUNEL (FIGS. 13J-13L) and activation of caspase-3 (FIG. 17), a hallmark of mitochondria-routed apoptosis (Davoli et al. 2002; Mohr et al. 1997), in experimental cerebral ischemia (Namura et al. 1998). Activation of caspase-3 involves its denitrosylation. It has been shown that caspase-3 remains inactivated in its nitrosylated form. The Fas apoptotic pathway has been shown to activate denitrosylation of caspase-3 (Mannick et al. 1999) leading to its activation. The treatment with GSNO in the inventor's model decreased the ischemia/reperfusion-induced activation of caspase-3 (FIG. 17) and reduced the number of TUNEL positive neurons as seen in FIGS. 13 and 16. To check whether the effect of GSNO is mediated through glutathione (GSH), the inventor treated the animals with exogenous GSH. The administration of GSH directly (up to 150 mg/kg body weight) after the onset of ischemia had no protective effect. Cerebral ischemia promotes activation of glial cells (resident microglia and astrocytes), and infiltration of blood-borne cells including neutrophils and macrophages (Stoll et al., 1998). Activated astrocytes (GFAP positive) and activated microglia/macrophages (ED-1 positive) were found in infarct and peri-infarct areas (FIG. 15). Because normal astrocytic function has been identified as critical for support of neuronal survival in acute stroke (Anderson et al., 2003), the abnormally in astrocytic metabolism including activation is considered to participate in injury. The infiltration of macrophage and neutrophils is dependent on endothelial function, which is compromised under hypoxic and ischemic conditions, and is preserved by NO and NO donors (Johnson et al., 1998). In a low or no oxygen/glucose conditions, eNOS is unable to provide the required NO for proper endothelial function. NO produced by eNOS in picomolar amounts is involved in preservation of endothelial function, cerebral blood flow and vasodilatation through guanylyl cyclase-cGMP pathway and/or nitrosylation/transnitrosylation of cysteine residue of proteins and small peptides (Tseng et al., 2000). In a reoxygenated ischemic brain with compromised endothelial functions, activated astrocytes and microglia/macrophages become the major source of iNOS and ROS in addition to cytokines and eicosanoids. Once induced, iNOS releases a burst of NO (nanomolar amounts) that may react with 0′ to form ONOO⁻. It is now clear that the role of NO is different in the presence of excessive ROS/0₂′ compared to the physiological levels of ROS/0₂⁻′ (Davis et al., 2001). In the absence of 0₂⁻′, NO may terminate the initiation and propagation of free radicals including lipid peroxide by several mechanism including regulation of enzymatic activity of lipid metabolizing enzymes, participation in cell signaling and binding to redox-active metal center to inhibit the generation of hydroxyl radicals. On the other hand, NO may either act in concert with ROS or react with ROS/0₂ to produce ONOO⁻ and HO′. The later two radicals are actively involved in corrupting DNA, proteins and lipids leading to cell death in ischemic injury. The protection provided by low dose of GSNO (1 mg/kg body weight) even after onset of focal cerebral ischemia indicates its ability to inhibit the activation of inflammatory cells (FIG. 15) and production of inflammatory mediators (FIG. 13). Inhibition of binding of subunits of NF- [symbol] B to DNA were certainly found in the in vitro studies. In addition to involvement of NF- [symbol] B, it is proposed that GSNO may exerted its anti-inflammatory effects through nitrosylation of the NMDA receptor and caspase-3. S-nitrosylation of NMDA (Lipton et al., 2002) receptor and caspase-3 (Mannick et al., 2001) is a well established mechanism of inhibition of injury in brain ischemia. Inhibition of iNOS expression in cell cultures via subunits p65/p50 of NF- [symbol] B clearly indicates that the neuroprotection does not depend on potential effects of GSNO on blood vessels and circulatory cells only, but it also exerts a direct anti-inflammatory effect. Further, the treatment in vivo with GSNO inhibited the ischemia/reperfusion-induced activity of caspase-3, thereby indicating the ability of GSNO to inhibit caspase-3 mediated cell death. Later, the window of treatment (up to 6 h) v/s protection provided by GSNO to brain from injury was checked in the model. GSNO remained highly protective when administered within 1.5 h, at a dose of 1 mg/kg body weight after the onset of ischemia in terms of infarction and neurological score (data not shown).

Regardless of the precise protective mechanism of GSNO against stroke, GSNO is a desirable neurorescue agent because it is easy to obtain and administer, is innocuous, and most importantly, is anti-apoptotic and anti-inflammatory. GSNO has been previously used and is well tolerated in both animals and human. Although GSNO in ischemia is anti-inflammatory and anti-apoptotic, the mechanism involved in neuroprotection by GSNO requires more studies, both in vivo and in vitro.

### Example 7

### Materials and Methods III

Cell culture and Reagents: Primary rat astrocytes and microglia were prepared from 1-3 day old postnatal Sprague-Dawley rat pups (McCarthy and de Vellis, 1980) and maintained in DMEM (4.5 gm glucose/L) with 10% fetal bovine serum (FBS) and antibiotics. Based on GFAP (glial fibrillary acidic protein) and MAC1 staining, astrocytes and microglia were more than 95% pure. Peritoneal macrophages were isolated and cultured in RPMI 1640 supplemented with heat inactivated 1% FBS medium (Pahan et al., 1997). BV2 is a microglia cell line derived from murine primary microglia provided by Dr. Michael McKinney (Mayo Clinic, Jacksonville, Fla.) and maintained in DMEM (4.5 gm glucose/L) supplemented with 10% FBS and antibiotics. DMEM (4.5 gm glucose/L), RPMI 1640 medium, fetal bovine serum and Hanks balanced salt solution were from Life Technologies (Grand Island, N.Y.). LPS (Escherichia coli, serotype 055:B5), GGPP, FPP, AICAR, mevalonate and protease inhibitor cocktail were from Sigma (St. Louis, Mo.). Antibodies against iNOS were obtained from Upstate (Waltham, Mass.). [γ-³²P] ATP (3000 Ci/mmol) and [γ-³²P]dCTP (3000 Ci/mmol) were from NEN (Boston, Mass.). Antibodies for p65, p50, IKKa, C/EBP-α, -β, -γ, -δ and oligonucleotides for NF-κB and C/EBP were from Santa Cruz (Santa Cruz, Calif.). Recombinant TNF-α, IL-1β, IFN-γ and ELISA kits for TNFα, IL-1β, IL-6 and IFN-γ were from R&D Systems (Minneapolis, Minn.). TRIZOL and transfection reagents (lipofectamine 2000, lipofectamine Plus and oligofectamine) were from Invitrogen (Carlsbad, Calif.). CAT ELISA, β-galactosidase, MTT and LDH kits were obtained from Roche (Nutley, N.J.). The enhanced chemiluminescence (ECL)-detecting reagents were purchased from Amersham Pharmacia Biotech (Arlington Heights, Ill.). Luciferase assay system was from Promega (Madison, Wis.). Gene expression arrays for inflammatory cytokines were from Superarray (Bethesda, Md.). Antibodies against phospho specific as well as nonphospho-p42/44, p38, JNK1/2 and AMPK were from Cell Signaling (Beverly, Mass.). NF- [symbol] B-luciferase, iNOS-Luciferase (3.2 kb) and AMPKα2 dominant negative expression vector (D157A) were kindly provided by Dr. W. J. Murphy, Dr. Zhang and Dr. David Carling, respectively. The expression vector for HA-IKKa was a gift from Dr. Zheng-Gang Liu. The iNOS (−1486/+145)-luciferase and iNOS-C/EBPdel-luciferase were kindly provided by Dr. Bruce C. Kone (Houston). The iNOS (−234/+31)-luciferase and iNOS (−331/+31NF-γ B mutated)-luciferase were kind gift from Dr. Mark A. Perrella (Boston).

Nitrite concentration: Synthesis of NO was determined by assay of culture supernatants for nitrite, a stable reaction product of NO with molecular oxygen as mention before (Pahan et al., 1997; Giri et al., 2002). Briefly, supernatants were mixed with an equal volume of the Griess reagent in 96 well plates, gently shaken and read in microplate reader at 570 nm. Nitrite concentrations were calculated from a standard curve derived from the reaction of NaNO₂ in the assay.

Immunoblot Analysis: Cells were harvested in ice-cold lysis buffer (50 mM Tris-HCl, pH 7.4, containing 50 mM NaCl, 1 mM EDTA, 0.5 mM EGTA, 10% glycerol and protease inhibitor cocktail) and protein was estimated using Bradford reagent (Bio-Rad, USA). Fifty microgram of total protein/lane was separated by SDS-PAGE and blotted to nitrocellulose (Amersham Pharmacia Biotech). Blots were blocked for 1 h in 5% nonfat dry milk-TBS-0.1% Tween 20 and incubated overnight with primary antibody (1:1000) in 5% BSA-TBS-0.1% Tween 20 at 4° C. This was followed by incubation of 1 h with appropriate secondary peroxidase conjugated antibody (1:10,000, Sigma). Immunoreactivity was detected using the enhanced chemiluminescence detection method according to the manufacturer's instructions (Amersham Pharmacia Biotech) and subsequent exposure of the membrane to X-ray film.

Fatty acid and cholesterol biosynthesis: Astrocytes grown in 6 well plate (˜80% confluency) and preincubated in serum-free media with AICAR for 2 h received [2-¹⁴C]acetate (5 μCi/well). After 2 h, the cells were washed twice with PBS and scraped off. Incorporation of labeled acetate in fatty acid and cholesterol was analyzed as mentioned earlier (Khan et al., 2000).

Antisense experiments: To decrease the levels of endogenous AMPK, astrocytes were pretreated for 48 h with 25 μm (of phosphothiorated antisense (AS) oligonucleotide (5′ CGCCCGTCGTCGTGCTTCTGC-3′) directly against both the [symbol] α1 and α2 [symbol] subunits of AMPK (Culmsee et al., 2001) [symbol] [symbol] [symbol] missense (MS) oligonucleotide (5′ CTCCCGGCTTGCTGCCGT-3′) was used in control cultures. Oligonucleotides were transfected with Oligofectamine™ reagent as per manufacturer's instructions.

AMPK and IKKα/ [symbol] assays: AMPK activity was assayed in primary rat astrocytes as described (Kim et al., 2001). For IKKα/ [symbol] assays, primary astrocytes were pretreated with AICAR (1 mM) and then stimulated with LPS (1 [symbol] g/ml-1) for 30 min. Cells were washed with cold PBS and lysed in lysis buffer (50 mM Tris-HCl, pH 7.4, containing 50 mM NaCl, 1 mM EDTA, 0.5 mM EGTA, 10% glycerol and protease inhibitor cocktail), Approximately, 200 μg of cell extracts was incubated with anti-IKKα/β antibody (Santa Cruz) for 2 h and then, added 30 μl of protein A/G PLUS agarose for further 1 h at 4° C. The immune complexes were washed twice in lysis buffer and twice in kinase buffer (20 mM HEPES, pH 7.5, 10 mM MgCl₂) and incubated at 30° C. in 30 μl of kinase buffer containing 20 mM β-glycerophosphate, 20 mM p-nitrophenyl phosphate, 1 mM dithiothreitol, 50 μM Na₃V0₄, 20 μM ATP, and 5 μCi of [γ-³²P] ATP. Approximately, 2 μg of GST-IκBα fusion protein (Santa Cruz) was used as substrate in each reaction. Reactions were stopped after 30 min by denaturation in SDS loading buffer. Proteins were resolved by SDS-polyacrylamide gel electrophoresis, and substrate phosphorylation was visualized by autoradiography.

Electrophoretic Mobility Shift Assay (EMSA): Nuclear extracts from stimulated or unstimulated astrocytes were prepared EMSA was performed as described previously (Giri et al., 2002) with NF-kB and C/EBP consensus sequences, which were end labeled with [γ-³²P] ATP. Nuclear extracts were normalized based on protein concentration and equal amount of protein (5 μg) was loaded. DNA-protein complexes were resolved on 5% non-denaturing PAGE in 45 mM Tris, 45 mM boric acid, 1 mM EDTA (0.5×TBE) and run at 11V/cm. The gels were dried and then autoradiographed at −70° C. using X-ray film.

Northern blot, gene array analysis and RT-PCR for cytokines expression: Total RNA was extracted with Trizol (Gibco) according to the manufacturer's protocol. Northern blot for iNOS was performed with 15 μg of RNA per reaction as described previously (Pahan et al., 1997). β-actin was used as control for RNA loading. Gene expression array for inflammatory cytokines (TNFα, IL-1β, IL-6 and IFN-γ) was used according to the manufacturer's protocol (Superarray). Signal quantitation was determined using imaging densitomter (Bio-Rad Lab, Hercules, Calif.). For RT-PCR, RNA was isolated from treated rat brain cerebral cortex by extracting in Trizol as above. cDNA was prepared from 5 μg of total RNA using poly dT as a primer and Moloney murine leukemia virus reverse transcriptase (Promega) as per manufacturer's instructions. 2 μl of cDNA was used to amplify the following products [given as product name, expected size, and forward (F) and reverse (R) primers used]: iNOS, 730 bp. (F) 5′-CTCCTTCAAAGAGGCAAAAATA-3′, (R) 5′-CACTTCCTC CAGGATGTTGT-3′; IL-1β, 623 bp. (F) 5′GCTGACAGACCCCAAAAGATT-3′, (R) 5′-TGTGCAGACTCAAACTCCACTT-3′; TNF-α, 473 bp. (F) 5′-CAGGGCAATGAT CCCAAAGTA-3′, (R) 5′-GCAGTGAGATCATCTTCTCGA-3′; GAPDH, 528 bp. (F) 5′-ACCACCATGGAGAAGGCTGG-3′, (R) 5′-CTCAGTGTAGCCCAGGATGC-3′. PCR products were visualized by electrophoresis in a 1.2% agarose gel containing 0.5 μg ethidium bromide and photographed with the UVP Bio-doc system (Upland, Calif.).

Cytokine assay. The levels of TNFα, IL-1β and IFN-γ were measured in culture supernatant as well as in serum by using enzyme linked immunosorbent assay (ELISA) using protocols supplied by the manufacturer (R&D Systems, MN).

Transcriptional assays: Primary astrocytes or microglial cell line (BV2) were transiently transfected with NF- [symbol] B—or iNOS-luciferase reporter gene with [symbol] -galactosidase in the presence or absence of dominant negative AMPKα2 or HA-IKK [symbol] by lipofectamine-2000 (astrocytes) and lipofectamine-Plus (BV2, Invitrogen) according to the manufacturer instructions. pcDNA3 was used to normalize all groups to equal amounts of DNA. Luciferase activity was determined using a luciferase kit (Promega).

Animals and LPS treatment: The use of animals was in accordance with the Guide for the Care & Use of Laboratory Animals (National Institute of Health, Pub. No. 86-23) and protocol approved by Medical University of South Carolina, Institutional Animal Care and Use Committee (IACUC). Female Sprague-Dawley rats (200-250 g; Jackson Laboratory, Bar Harbor, Me.) were group housed at room temperature under 12 h:12 h light:dark conditions with ad libitum food and water. Animals were injected intraperitoneally (i.p.) with AICAR (100 mg/kg. body weight) 30 min prior to LPS treatment (0.5 mg/kg. body weight) dissolved in 0.9% saline or 0.9% sterile saline alone. After 6 h, cerebral cortex was isolated and frozen in liquid nitrogen followed by 70° C. until further use.

Cell Viability: Cytotoxic effects of treatments were determined by measuring the metabolic activity of cells with 3-(4,5-dimethyl thiazol-2-yl)-2,5-diphenyl tetrazolium bromide (MTT) and LDH release assay (Roche).

Statistical analysis: Results shown represent means ±S.D. Statistical analysis was performed by ANOVA by the Student-Neuman-Keuls test using GraphPad InStat software (San Diego, Calif.).

### Example 8

### Results III

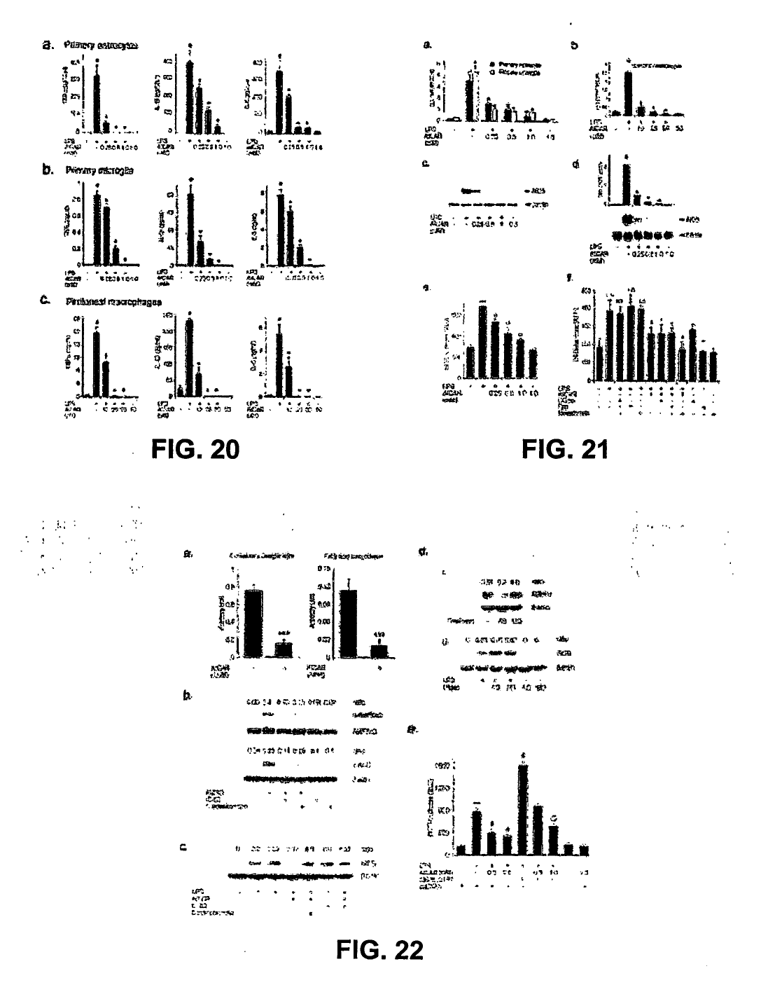

AICAR down regulates LPS-induced expression of pro-inflammatory cytokines in brain glial cells and peritoneal macrophages: Activated astrocytes, microglia and macrophages are the major sources of NO and cytokines production and actively participate in inflammatory disease (Benveniste, 1997). Rat primary astrocytes, microglia and peritoneal macrophages were pretreated with different concentrations of AICAR and then exposed to LPS (1 μg/ml). Bacterial LPS markedly induced the production of pro-inflammatory cytokines (TNFα, IL-1β and IL-6) in astrocytes (FIG. 20a), microglia (FIG. 20b) and macrophages (FIG. 20c) determined by ELISA. AICAR alone had no effect on the production of cytokines; however, it strongly inhibited the LPS-induced production of TNFα, IL-1β and IL-6 in the supernatants of these cells in a dose dependent manner (FIG. 20). Inhibition in cytokine production was accompanied by decreased mRNA expression (data not shown). Interestingly, macrophages were more sensitive to AICAR as micromolar concentration of AICAR was sufficient to inhibit pro-inflammatory cytokines production as compared to astrocytes and microglia (FIG. 20c). AICAR or LPS had no effect on cell viability as tested by LDH and MTT assays (data not shown).

AICAR inhibits LPS-induced NO production and iNOS gene expression in brain glial cells: Along with the production of pro-inflammatory cytokines, NO production in response to cytokines has been shown to be important in the pathophysiology of a number of inflammatory diseases (Smith et al., 1999). Rat primary astrocytes, microglia and macrophages were pretreated with different concentrations of AICAR and then exposed to LPS (1 μg/ml). LPS induced NO production (measured as nitrite) 10-fold higher as compared to untreated cells. AICAR treatment inhibited LPS-induced nitrite production in primary astrocytes, microglia and peritoneal macrophages in a dose dependent manner (FIGS. 21A and B). Similar to cytokine production, macrophages were more sensitive to AICAR treatment compared to primary astrocytes and microglia (FIG. 20c).

To understand the inhibitory mechanism of AICAR on LPS-mediated nitrite production, the effect of AICAR on iNOS protein and mRNA level in primary rat astrocytes was examined. Consistent with the production of nitrite, LPS-induced expression of iNOS was inhibited by AICAR at the mRNA as well as the protein levels (FIGS. 21C and D). The inventor next examined the activation of iNOS promoter in primary astrocytes in response to LPS treatment and the effect of the AICAR on that activity. A plasmid containing a 3,2-kb portion of the rat iNOS promoter attached to the luciferase gene (iNOS-Luc) was introduced into sub-confluent cultures of primary astrocytes by transient transfection. After 24 h, the cultures were pretreated with different concentration of AICAR (0.25 to 1 mM) followed by LPS treatment for further 6 h (FIG. 21E). The iNOS promoter activity was substantially (2.5-fold) stimulated upon incubation with LPS (FIG. 21e). However, it significantly inhibited by AICAR treatment in a dose-response manner. Previous reports (Hardie, 1992) as well as the inventor's experiment demonstrates that AICAR significantly inhibits cholesterol biosynthesis in primary astrocytes (FIG. 22a). Further, the inventor investigated, if any intermediate(s) or metabolite(s) of cholesterol biosynthesis pathway may be responsible for anti-inflammatory effect of AICAR. Addition of mevalonate, geranylgeranyl pyrophosphate (GGPP) and farnesyl pyrophosphate (FPP) did not reverse the inhibitory effect of AICAR on LPS induced iNOS-luciferase activity in primary astrocytes (FIG. 21f).

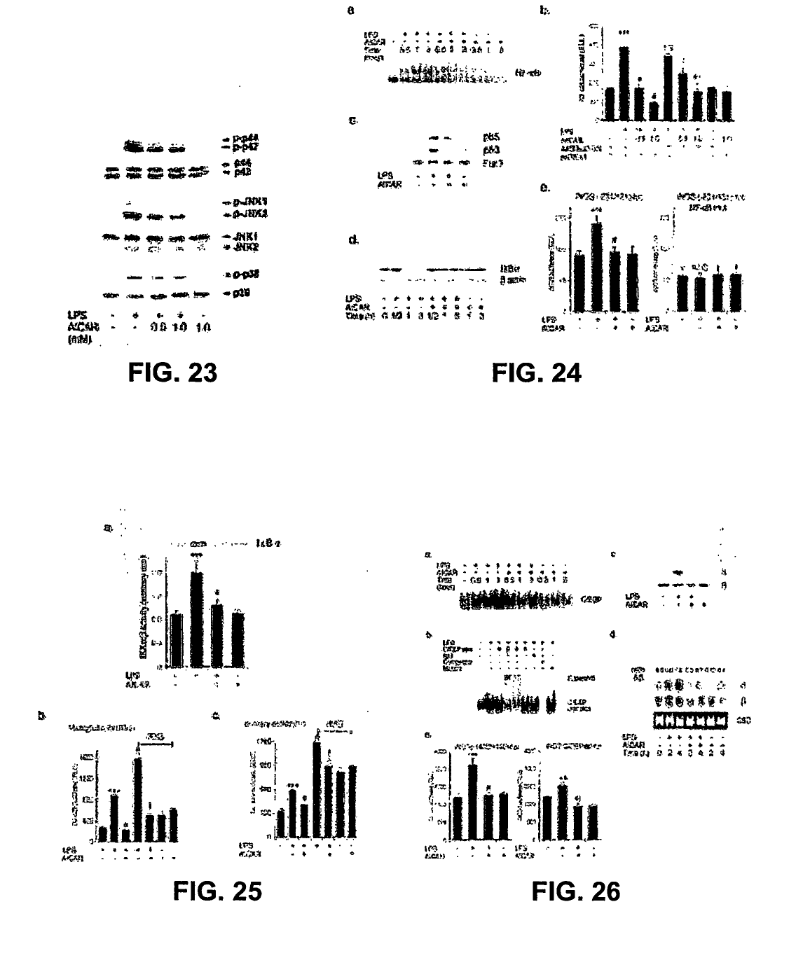

AICAR inhibits iNOS gene expression via activation of AMP-activated protein kinase (AMPK): AICAR mediates its effects by activating AMPK; it was imperative to establish the role of AMPK in regulation of the inflammatory process. Once activated, AMPK down regulates ATP consuming pathways such as fatty acid and cholesterol synthesis by phosphorylating acetyl-CoA-carboxylase (ACC) and HMG-CoA reductase the inventor observed the same when treatment of primary astrocytes with AICAR (1 mM) resulted in a significant inhibition of cholesterol and fatty acid biosynthesis (˜80%) (FIG. 22a). Treatment of astrocytes with AICAR also induced the phosphorylation of ACC (FIG. 22b) demonstrating that AICAR induced AMPK activity. AMPK is not only allosterically activated by AMP, but is also the target of the upstream kinase AMP-activated protein kinase kinase (AMPKK), and the phosphorylation of AMPK by AMPKK is necessary for its full activity (Moore et al., 1991; Hawley et al., 1996; Hardie et al., 1998; Stein et al., 2000). Phosphorylation of AMPK in AICAR treated astrocytes clearly demonstrating that AICAR not only induced AMPK activity (induced phosphorylation of ACC) but also induced upstream kinase (AMPKK) activity (to induce phosphorylation of AMPK) (FIG. 22b). Treatment of astrocytes with AICAR induced the phosphorylation of AMPK and ACC (FIG. 23b), which was blocked by 5-iodotubercidin and IC51, inhibitors of adenosine kinase (ADK) that blocks the formation of ZMP (5′-phosphorylated form of AICAR) from AICAR. ZMP mimics the effect of AMP in the allosteric activation of AMPK without altering the levels of AMP, ADP and ATP (Corton et al., 1995). AICAR induced phosphorylation of AMPK can be blocked by ADK inhibitors (FIG. 22b) and in turn reverse the inhibitory effect of AICAR on LPS induced iNOS protein expression (FIG. 22c).

To further elucidate the role of AMPK in regulating iNOS expression, the inventor employed an antisense oligonucleotide against a sequence near the translation initiation site of the mRNA encoding the catalytic subunits (AMPKα1 and α2 [symbol] [symbol] of AMPK (Culmsee et al., 2001). Transfection with 25 μM phosphothiorated antisense (AS) of primary astrocytes decreased the catalytic subunit AMPKα1 whereas missense (MS) had no effect [FIG. 22d (i)]. Next, the inventor examined the effect of the AMPKα2 subunit antisense oligonucleotide on LPS mediated iNOS gene expression. AMPKα2 subunit antisense treatment alone did not have any effect on iNOS expression but it significantly induced the LPS mediated iNOS protein expression in primary astrocytes [FIG. 22d (ii)]. These experiments provide clear evidence of involvement of AMPK in the regulation of inflammatory responses. In order to define a direct role of AMPK in the inflammatory process and to decipher the molecular mechanism/pathway of AMPK in this function, microglial cell line (BV2) was employed. This cell line derived from mouse primary microglia, is ease of transfection and produces the pro-inflammatory cytokines and mediators in response to LPS (Kim et al., 2002; Su et al., 2003). In transient transfection studies, LPS significantly induced (˜4 fold) iNOS-luc activity in BV2 cell line and pretreatment of AICAR in a dose-dependent manner decreased the luciferase activity (FIG. 22e). In co-transfection experiments, the inventor tested the effect of expression of dominant negative (DN) AMPK α2 [symbol] [symbol] D157A [symbol] [symbol] (Stein et al., 2000) on iNOS promoter activity. DN AMPK α2 not only resulted in a significant increase in LPS induced iNOS-Luc activity but also significantly reversed the AICAR induced inhibition in iNOS-Luc activity (FIG. 22e). These experiments clearly demonstrate a correlation between AMPK and the expression of inflammatory mediators in brain glial cells.

Mitogen activated protein kinases (ERK1/2, p38 and JNK1/2) are known to play a regulatory role in the expression of pro-inflammatory mediators (Arbabi and Maier, 2002). Therefore, the effect of AICAR on LPS-mediated activation of ERK1/2, JNK1/2 and p38 MAPKs in rat primary astrocytes was investigated. Consistent with the documented role of MAPKs, LPS induced the phosphorylation of all three MAPKs, whereas AICAR inhibited the LPS mediated phosphorylation of these kinases by 40-60% (FIG. 23). AICAR alone had no effect on the phosphorylation of these MAP kinases.

AICAR attenuates the inflammatory response by inhibiting nuclear translocation of LPS-induced NF-κB and C/EBP: To understand the mechanism of AICAR mediated downregulation of the inflammatory process, the inventor investigated the effect of AICAR on LPS mediated NF-κB activation. In unstimulated cells, NF-kB consists of a p65/p50 heterodimer and is retained in cytoplasm by its association with IκB. After stimulation of cells with various agents, the cytosolic NF-κB/IκB complex dissociates and free NF-κB translocates to nucleus and regulates the transcription of various genes. Phosphorylation of IkBα by the upstream kinase IKK is essential for the dissociation of IκBα from NF-κB and its degradation (Ghosh and Karin, 2002). Activation of NF-κB has been shown to be critical for the expression of iNOS and pro-inflammatory cytokines (TNFα and IL-6) (Zagariya et al., 1998; Zhang et al., 1998; Hu et al., 2000). The role of AICAR was investigated in LPS mediated activation of NF-κB in primary astrocytes. As shown in FIG. 24a, LPS treatment activates and translocates NF-κB into nucleus within 30 min and this is sustained up to 3 h after treatment. Pretreatment with AICAR significantly reduced the LPS induced DNA binding activity of NF-κB (FIG. 24a). The possible role of AMPK in the regulation of NF-κB transcriptional activity was investigated by co-transfecting a NF-κB-dependent transcriptional reporter (3×NF-κB-luc) with an expression vector encoding dominant negative AMPK in BV2 cells. As depicted in FIG. 24b, AICAR pretreatment significantly inhibited LPS induced NF-κB-Luc activity. Although, the dominant negative AMPKα2 had no effect on LPS induced NF-κB-Luc activity but it significantly reversed the AICAR induced inhibition (FIG. 24b). AICAR induced inhibition in LPS mediated NF-κB nuclear translocation was consistent with the results of immunoblot analysis of nuclear extracts for p65 and p50 (members of the NF-κB family)(FIG. 24c). Moreover, these conclusions are further supported by the inhibition of degradation of IkBα [symbol] by AICAR treatment (FIG. 24d).

In addition to this, microglial cells (BV2) were transfected with the iNOS-luci (−234/+31) vector, a construct strictly dependent on NF-kB activation. As FIG. 24e shows, AICAR completely abolished the luciferase activity induced by LPS treatment. Interestingly, the use of a fragment of the NOS-2 promoter deleted in the k-luci] completely abolished the activity of the promoter, reflecting the necessity of this motif for expression of the reporter gene in response to LPS stimulation (FIG. 24e) and AICAR mediates its effect via down regulating NF-κB pathway.

IκBα is phosphorylated by the IKK complex containing catalytic subunits (IKK a [symbol] & β) and the IKKγ or NEMO regulatory subunits, at sites that trigger its ubiquitin-dependent degradation (Ghosh and Karin, 2002). To determine whether IKKa/β could be the target for AICAR action, astrocytes were pre-incubated with AICAR (1 mM) followed by LPS treatment. As documented in FIG. 25a, LPS stimulated the IKKα/β activity and this stimulation was significantly blocked by AICAR treatment. To confirm these observation, the inventor co-transfected wild type expression vector of IKKβ [symbol] with NF-κB-Luc in microglial cell (BV2) and primary astrocytes cells. AICAR treatment significantly inhibited IKKβ [symbol] mediated NF-κB-Luc activity in BV2 cells and primary astrocytes (FIGS. 25b and c). These observations clearly demonstrate that AICAR inhibits NF-κB DNA binding as well as its transcriptional activity by inhibiting some unknown upstream molecule(s) of IKKs.

In addition to NF-κB, C/EBP-binding motifs have been identified in the functional regulatory regions of various pro-inflammatory genes such as IL-6, IL-1β, TNFα, IL-8, IL-12, granulocyte colony stimulating factor (G-CSF), iNOS, lysozyme, myeloperoxidase, neutrophil elastase and granulocyte-macrophage receptor (Poli, 1998). Therefore, C/EBP DNA binding activity was examined by EMSA at different time periods (varying from 0.5 to 3 h) in primary astrocytes treated with LPS and/or AICAR. LPS induced the nuclear translocation of C/EBP and AICAR abolished the LPS induced C/EBP DNA binding activity whereas AICAR alone had no effect on the nuclear translocation of C/EBP (FIG. 26a). Further, to identify the C/EBP protein(s) responsible for the C/EBP complex, supershift assays with antibodies specific for C/EBP-α, -β, -δ and -ε were performed. Only the IgGs specific for C/EBP-β and -δ significantly super shifted C/EBP complex (FIG. 26b). The nuclear translocation of C/EBP-β and -δ was examined by immuno blot of nuclear extracts of LPS and LPS/AICAR treated primary astrocytes. C/EBP-β was constitutively expressed and localized in the nucleus of untreated cells and its level was not modulated with LPS and/or AICAR (FIG. 26c). On the other hand, high levels of C/EBP-δ were observed in the nuclear extract of LPS treated cells as compared to untreated cells and translocation of C/EBP-δ was completely inhibited by AICAR treatment (FIG. 26c). It was of interest to examine whether AICAR inhibited the translocation of C/EBP δ into the nucleus or its expression in primary rat astrocytes. For this, the inventor examined the expression of C/EBP-δ in primary astrocytes treated with LPS and AICAR. LPS induced mRNA expression of C/EBP-δ and AICAR attenuated the LPS mediated C/EBP-δ expression (FIG. 26d). To demonstrate that C/EBP plays an important role in the regulation of iNOS gene expression in glial cells, the inventor employed iNOS2 promoter lacking the −150 to −142 C/EBP box (iNOS-C/EBPdel-luc). Microglial (BV2) cells transfected with iNOS-luc and treated with LPS exhibited significant increase in normalized luciferase activity and AICAR treatment completely abolished this luciferase activity (FIG. 26e). In contrast, cells transfected with iNOS-C/EBPdel-luc generated slight increase in normalized luciferase activities after LPS stimulation (FIG. 26e). These results indicated the role of C/EBP in regulation of iNOS gene expression in response to LPS. Taken together, the observations document that AICAR inhibited the LPS-induced C/EBP nuclear translocation by down regulating the expression of C/EBP δ.

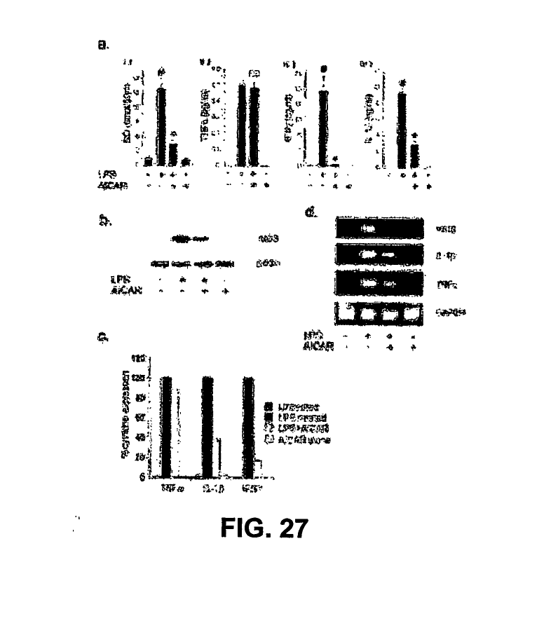

AICAR inhibits the production of pro-inflammatory cytokines and nitrite in LPS-treated rats: Since AICAR exhibited anti-inflammatory properties in cultured cell (by inhibiting the nuclear translocation of NF-κB and C/EBP), it was of further interest to examine the same effect of AICAR in vivo. It is well established that expression of pro-inflammatory cytokines, such as TNFα, IL-1β and IFN-γ, are induced by intraperitoneal injection of LPS in vivo (Hesse et al., 1988). Therefore, the inventor examined the effect of AICAR on serum cytokines levels in LPS injected rats. The levels of TNFα, IL-1β and IFN-γ were measured in serum 6 h post LPS injection. As shown in FIG. 27a, LPS efficiently induced pro-inflammatory cytokines (TNFα, IL-1β and IFN-γ) whereas pretreatment with AICAR almost abolished LPS mediated increased levels of IL-1β and IFN-γ in serum. However, it had no effect on the levels of TNFα. AICAR treatment also significantly inhibited LPS induced expression of iNOS in peritoneal macrophages isolated from these animals (FIG. 27b). Further, the inventor examined the effect of AICAR on expression of these cytokines in spleen by gene array analysis. Similar to the observations in serum, intraperitoneal injection of LPS significantly induced the expression of TNFα, IL-1β and IFN-γ message in spleen (FIG. 27c). The mRNA expression of IL-1β and IFN-γ was significantly decreased by AICAR while no significant change was observed in the expression of TNFα in spleen (FIG. 27c). Neither saline nor AICAR alone induced a detectable signal for these cytokines.

Models of peripheral immune challenge or peripheral inflammation have been shown to induce the expression of pro-inflammatory cytokines within the brain (Pitossi et al., 1997), therefore, the inventor examined the expression of TNFα, IL-1β and iNOS in cerebral cortex of LPS injected rats treated or untreated with AICAR. LPS induced expression of TNFα, IL-1, and iNOS in the cerebral cortex while AICAR treatment significantly reduced the expression of these molecules (FIG. 27d). These findings document that similar to cultured glial cells, AICAR was also effective in attenuating the expression of pro-inflammatory molecules (except TNFα) in an animal model (FIG. 27).

### Example 9

### Discussion III

AMP-activated protein kinase (AMPK) was originally identified through its ability to phosphorylate and inhibit the key enzymes involved in biosynthetic pathways, such as acetyl CoA-carboxylase (fatty acid synthesis) and HMG CoA-reductase (isoprenoid and cholesterol biosynthesis) (Moore et al., 1991; Vincent et al., 1991; Hardie and Carling, 1997; Hardie et al., 1998; Winder and Hardie, 1999). Since cholesterol metabolites have been recently reported to attenuate the inflammatory process (Pahan et al., 1997; Kwak et al., 2000), the inventor examined the possible role of AMPK in the induction of the inflammatory process in cultured cells as well as in LPS injected animals. Several lines of evidence presented in this manuscript clearly support the conclusion that activation of AMPK by AICAR down regulates LPS mediated induction of pro-inflammatory cytokines, iNOS and nitric oxide (NO) production in rat primary astrocytes, microglia and peritoneal macrophages by inhibiting the nuclear translocation of NF-κB and C/EBP transcription factors, thereby demonstrating the involvement of AMPK in the regulation of expression of inflammatory mediators. This study also suggests the therapeutic use of AICAR or other pharmacological activators of AMPK for inflammatory diseases. Although AICAR inhibits pro-inflammatory cytokines in tissue culture and CNS of LPS injected rats but why it did not affect LPS induced TNF [symbol] [symbol] levels [symbol] in serum, can't be explained at this time.

AICAR induced the phosphorylation and activation of AMPK and inhibition of ACC and HMG-CoA reductase suggesting the activation of AMPK and its upstream kinase (AMPKK). HMG-CoA reductase inhibitors such as statins have been reported to be immunomodulatory and anti-inflammatory (Pahan et al., 1997; Kwak et al., 2000). However, the inventor found that the mechanism of action of AICAR/AMPK is not through the mevalonate pathway since addition of mevalonate and other metabolites did not reverse the inhibitory effect of AICAR on LPS-induced NO production. Since MAPKs are known to play an important role in the expression of pro-inflammatory molecules such as TNFα, IL-1β, IL-6, IL-8, COX-2 and iNOS (Arbabi and Maier, 2002), the observed inhibition of LPS-induced activation of all three MAPKs (ERK1/2, p38 and JNK1/2) by AICAR indicates a role for AMPK in the regulation of these signaling pathways. AMPK has been reported to regulate the endothelial growth factor (EGF) and insulin growth factor (IGF) mediated ERK pathway by phosphorylation of Raf-1 Ser621 (Sprenkle et al., 1997; Kim et al., 2001). In contrast to the inventor's observations, the activity of p38 MAPK has been shown to be activated by AMPK in a rat liver derived nontransformed cell line (Xi et al., 2001). This may be one of the cell specific functions of AMPK.

In the inventor's experimental conditions, the specificity of AICAR to activate AMPK is documented by number of experiments as follows; i.) Dominant negative form of AMPK [symbol] [symbol] [symbol] reversed the AICAR induced inhibition in iNOS- and NF- [symbol] B-luciferase activity. ii.) Inhibitors of adenosine kinase (5′-iodotubercidin and IC51) were not only able to reverse the inhibition induced by AICAR on iNOS protein expression but also inhibited the AICAR induced phosphorylation of AMPK and ACC. iii.) Down regulation of catalytic subunits of AMPK by antisense oligonucleotides induced the expression of iNOS protein levels in primary astrocytes. Recently, AMPK [symbol] 2 knockout mice have been reported (Viollet et al., 2003) and these studies in those mice will definitely define the role of AMPK in the regulation of inflammatory cytokines.

The possibility that AMPK is a component of the transcriptional regulatory complexes is yet to be explored. Recently, p300, a transcriptional coactivator has been reported to be a substrate of AMPK in vivo and in vitro and upon phosphorylation its interaction with other nuclear receptors such as PPARγ, thyroid receptor, RAR and RXR were dramatically reduced (Yang et al., 2001). the inventor's study clearly demonstrated that AMPK regulates the transcriptional activity of NF-κB and C/EBP. The activation of AMPK inhibits nuclear translocation as well as transcriptional activity of NF-κB by inhibiting LPS-induced IKKα/ [symbol] activity and phosphorylation/degradation of IκBα indicating that AMPK targets the NF-κB pathway upstream of IKKs. On the other hand, AICAR not only inhibited nuclear translocation of C/EBP but also down regulated the LPS-induced expression of C/EBP-δ in primary astrocytes. These observations identify the C/EBP pathway as one of the potential candidates for therapeutics against inflammatory disease since C/EBP is known to regulate the expression of TNFα, IL-1β, IL-6, iNOS, IL-8, IL-12 and GM-CSF (Poli, 1998).

Since, pro-inflammatory cytokines (TNFα, IL-1β and IL-6) and NO have been implicated in the pathogenesis of demyelinating and neurodegenerative diseases (Benveniste, 1997; Smith et al., 1999; Torreilles et al., 1999; Bauer et al., 2001), the inventor's results provide a potentially important mechanism whereby an activator of AMPK may prevent or ameliorate neural injury. AMPK is a heterotrimeric protein kinase consisting of a catalytic α subunit and noncatalytic β and γ subunits (Hardie and Carling, 1997; Hardie et al., 1998; Winder and Hardie, 1999). There are different isoforms for each subunit, termed α1 or α2, β1 or β2 and γ1, γ2 or γ3 that have been described (Kemp et al., 1999). Immunostaining for AMPK documented that the expression of α2 AMPK subunit in brain was confined mainly to neurons and white matter astrocytes (Turnley et al., 1999). Normally, most astrocytes express low levels of AMPK, but its expression increases (mainly [symbol] 2 and [symbol] 2) when there is an increase in metabolic activity, such as during reactive gliosis (Turnley et al., 1999), in which astrocytes become enlarged, migrate to the site of injury and release a variety of cytokines and growth factors. The observed higher expression of AMPK in reactive astrocytes and identification of co-localization of AMPK isoforms in the nucleus indicate that AMPK may also have other functions in addition to the regulation of energy metabolism (Salt et al., 1998; Turnley et al., 1999). These findings are consistent with the role of AMPK in the inflammatory process reported in this study. AMPK has been recently reported to have a protective function during glucose deprivation in neurons (Culmsee et al., 2001) and has been shown to protect astrocytes (Blazquez et al., 2001) and thymocytes (Stefanelli et al., 1998) from apoptosis and necrosis. Recently, a novel function of AMPK in neurodegeneration and APPL/APP processing has been demonstrated in Drosophila, which could be mediated through HMG-CoA reductase and cholesterol ester (Tschape et al., 2002). All these observations suggesting a crucial role of AMPK in CNS and supporting the inventors study. It strongly indicates that AMPK plays an important role, as an anti-inflammatory molecule and may be exploited as a target molecule for anti-inflammatory drugs such as AICAR. Moreover, AICAR has been previously used as a drug for treating Lesch-Nyhan syndrome at a relatively high dose (100 mg/kg body weight) safely and without any side effects (Page et al., 1994). The safety, tolerance and pharmacokinetics of intravenous doses of 10-100 mg/kg of AICAR in health men have previously been reported (Dixon et al., 1991). AICAR has a high clearance and is poorly bioavailable with oral administration.

In summary, the studies described in this manuscript document a novel role of AMPK in inflammatory disease. AMPK may be an interesting target for neuroprotective drugs in inflammatory conditions such as multiple sclerosis, Alzheimer's, stroke and other neurodegenerative diseases.

### Example 10

### Statins as Therapeutics for Inflammatory Diseases

This example provides, among other things, data showing that statins can be used to treat or prevent inflammatory diseases. Table 1, for example, shows that the combination of Lovastatin and an inhibitor of FPP decaroxylase (e.g., NaPA) inhibits LPS-induced production of nitric oxide, TNFα, II-1β, and IL-6 in Rat Primary Astrrocytes, Microglia, and Macrophages.

TABLE 1*

| | Production | | | |
|---|---|---|---|---|
| | of NO or | | LPS + | |
| Cells | Cytokines | LPS Only | Lovastatin | LPS + NaPA |
| Astrocytes | NO | 25.3 +/− 3.2 | 5.2 +/− 0.4 | 5.4 +/− 0.6 |
| | TNF-α | 5.3 +/− 0.8 | 0.3 +/− 0.05 | 0.4 +/− 0.06 |
| | Il-1β | 10.4 +/− 1.5 | 0.8 +/− 0.1 | 1.1 +/− 0.2 |
| | IL-6 | 136.5 +/− 16.8 | 0.8 +/− 0.1 | 1.1 +/− 0.2 |
| Microglia | NO | 81.2 +/− 6.9 | 5.9 +/− 0.4 | 6.9 +/− 0.9 |
| | TNF-α | 14.5 +/− 2.1 | 0.9 +/− 0.1 | 1.3 +/− 0.2 |
| | Il-1β | 28.2 +/− 3.4 | 2.1 +/− 0.3 | 2.4 +/− 0.2 |
| | IL-6 | 295.6 +/− 33.5 | 7.8 +/− 1.1 | 9.3 +/− 1.2 |
| Macro- | NO | 118.5 +/− 12.5 | 7.2 +/− 0.9 | 9.5 +/− 0.7 |
| phages | TNF-α | 18.6 +/− 2.3 | 1.2 +/− 0.1 | 1.7 +/− 0.2 |
| | Il-1β | 34.6 +/− 4.5 | 2.3 +/− 0.3 | 3.1 +/− 0.4 |
| | IL-6 | 350.0 +/− 27.6 | 8.3 +/− 0.6 | 10.2 +/− 1.4 |

*Cells preincubated with 10 μM lovastatin or 5 mM NaPA for 8 h in serum free condition was stimulated with 1.0 μg/ml of LPS. After 24 h of incubation, concentration of NO, TNF-α, IL-1β and IL-6 were measured in supernatants as mentioned above. NO is expressed as n mol/24 h/mg protein whereas TNF-α, IL-1β and IL-6 are expressed as ng/24 h/mg protein. Data are expressed as the mean =/− SD of three different experiments.

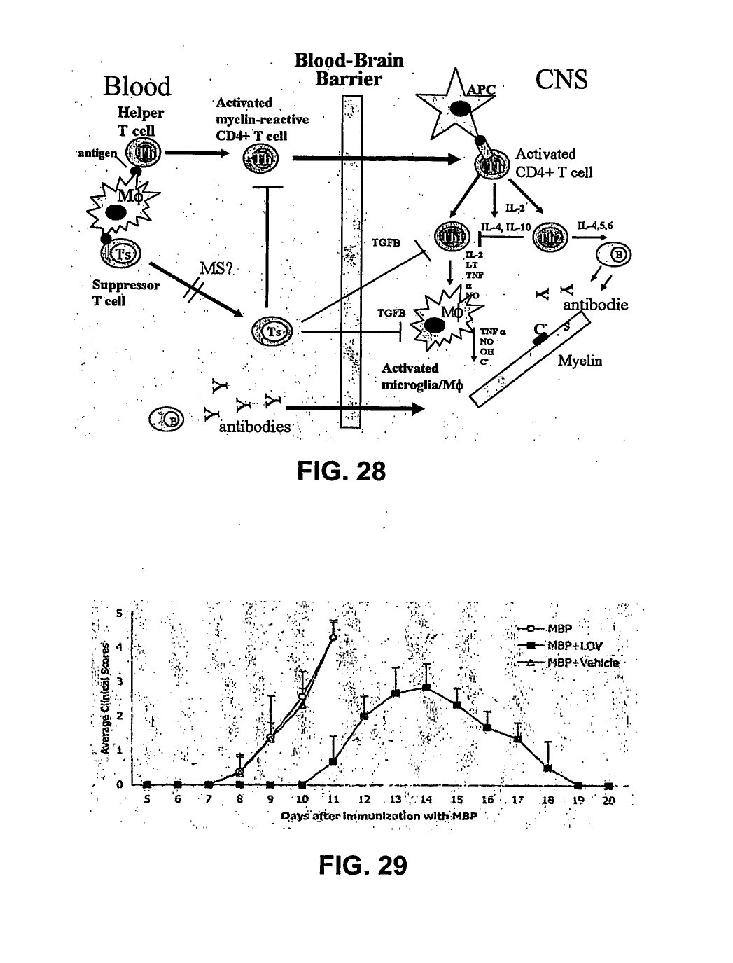

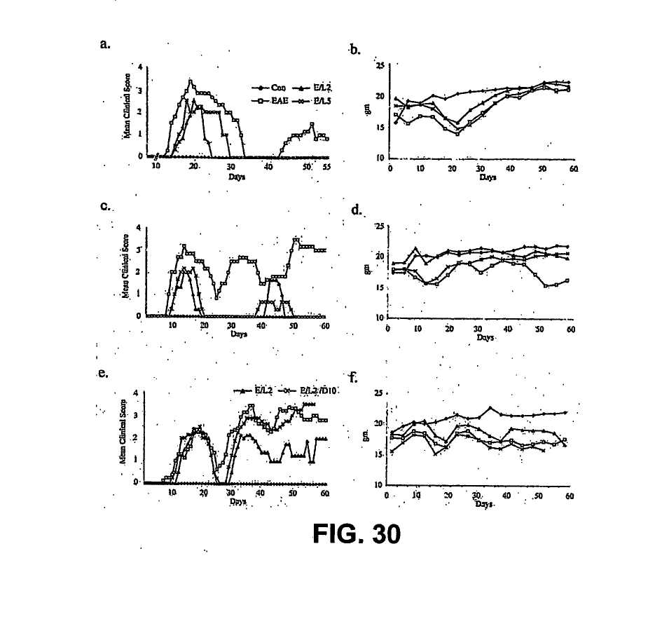

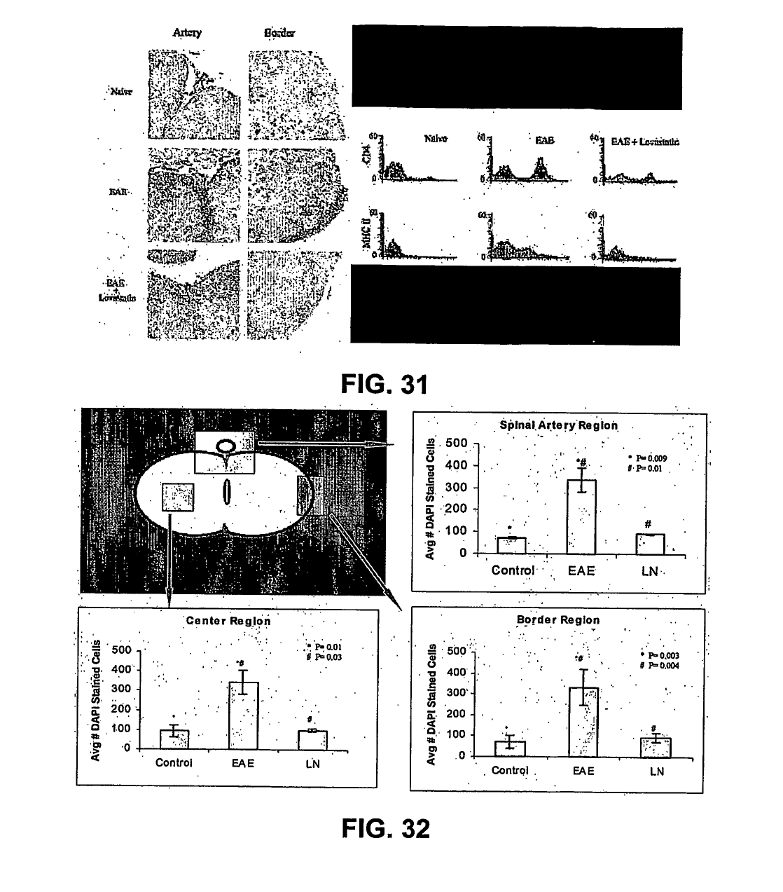

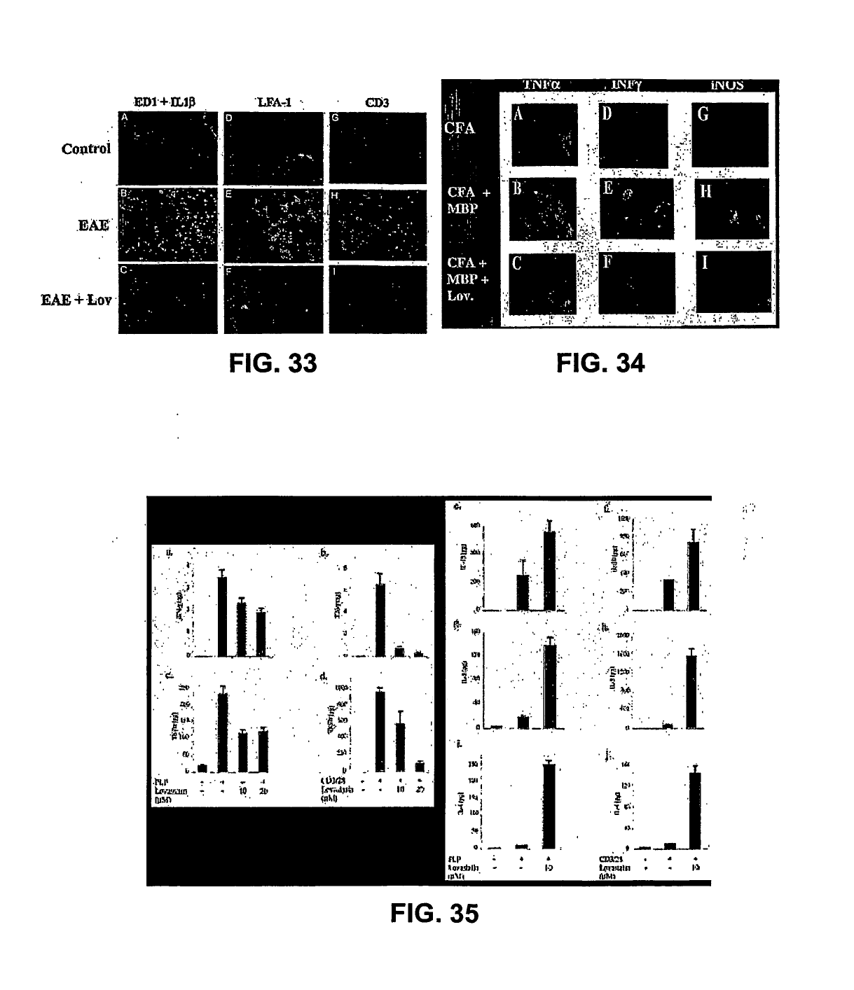

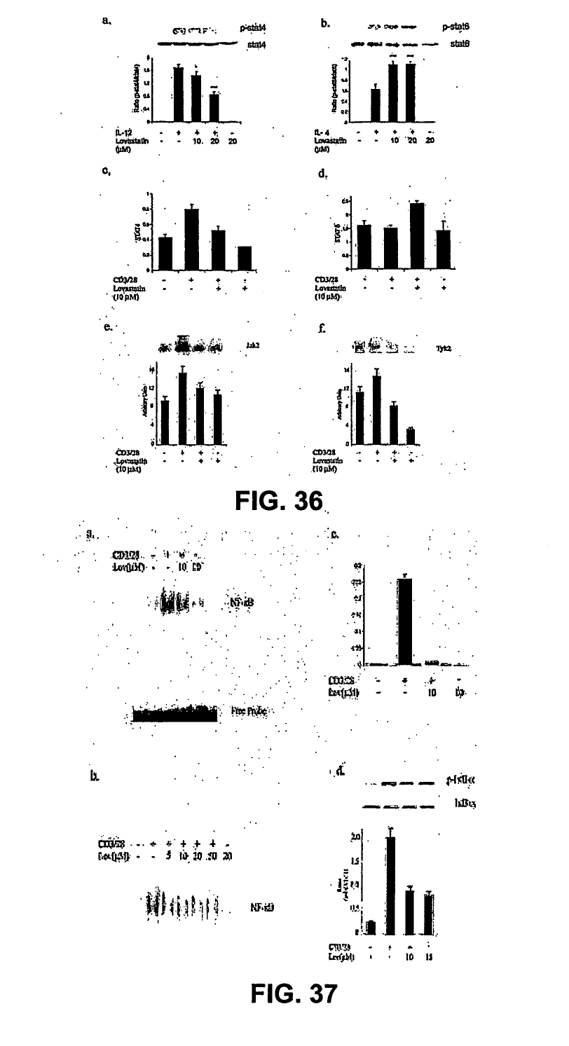

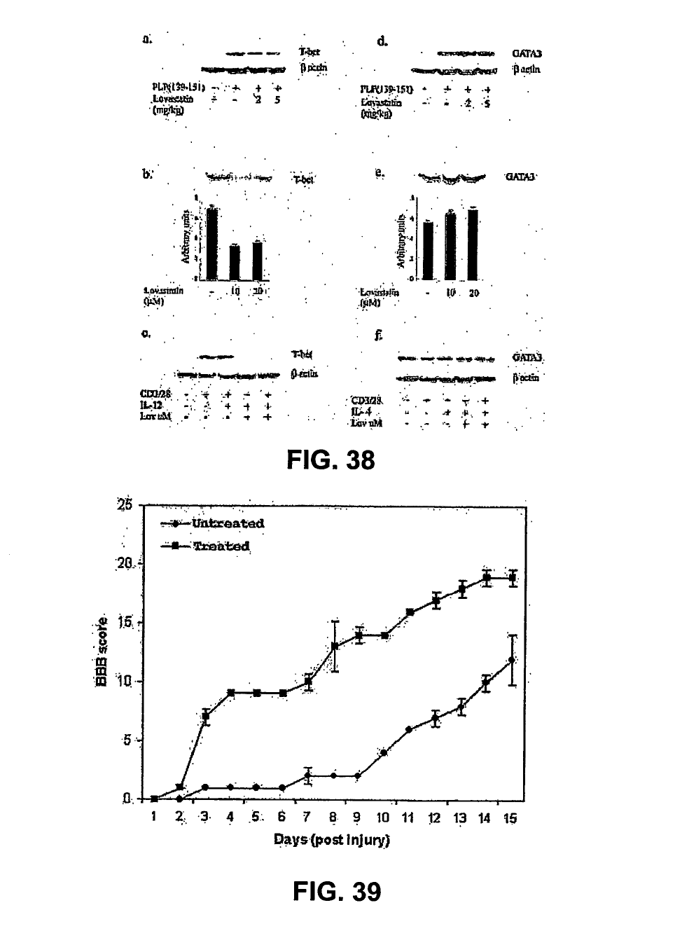

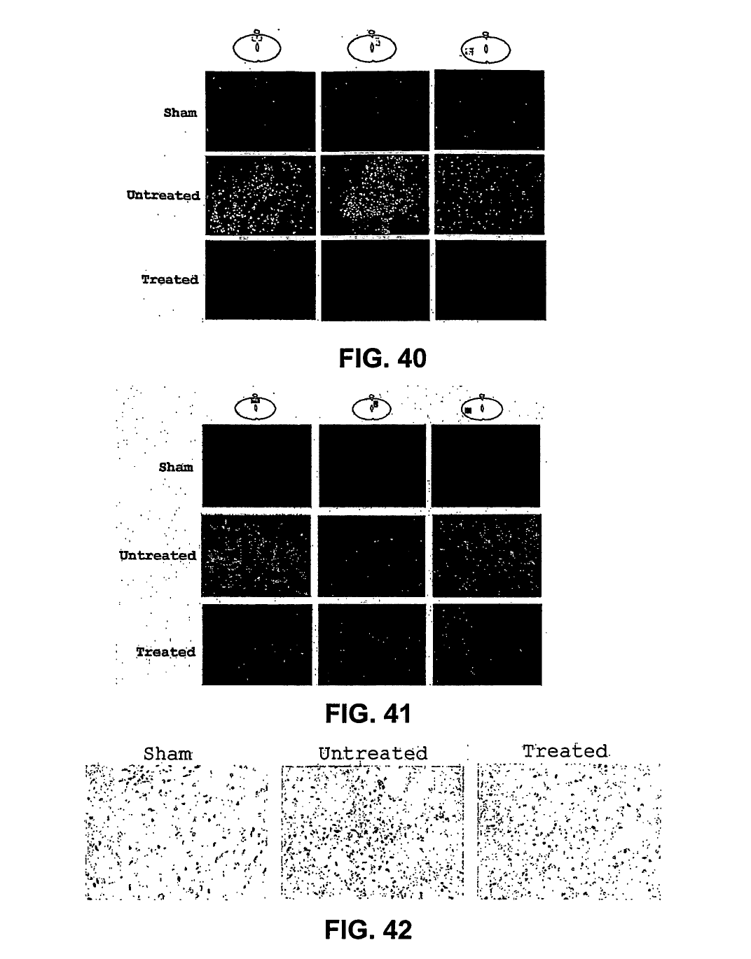

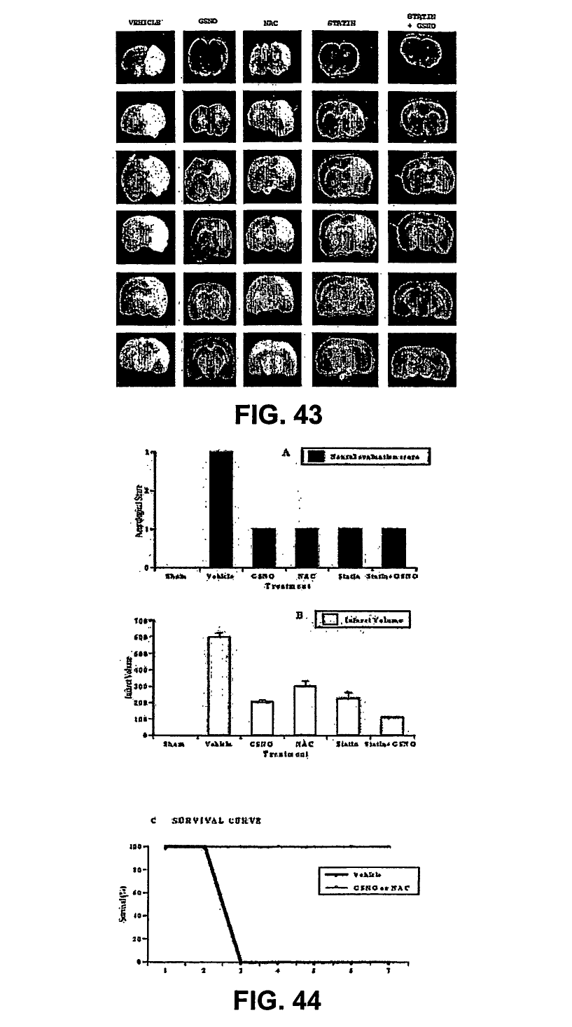

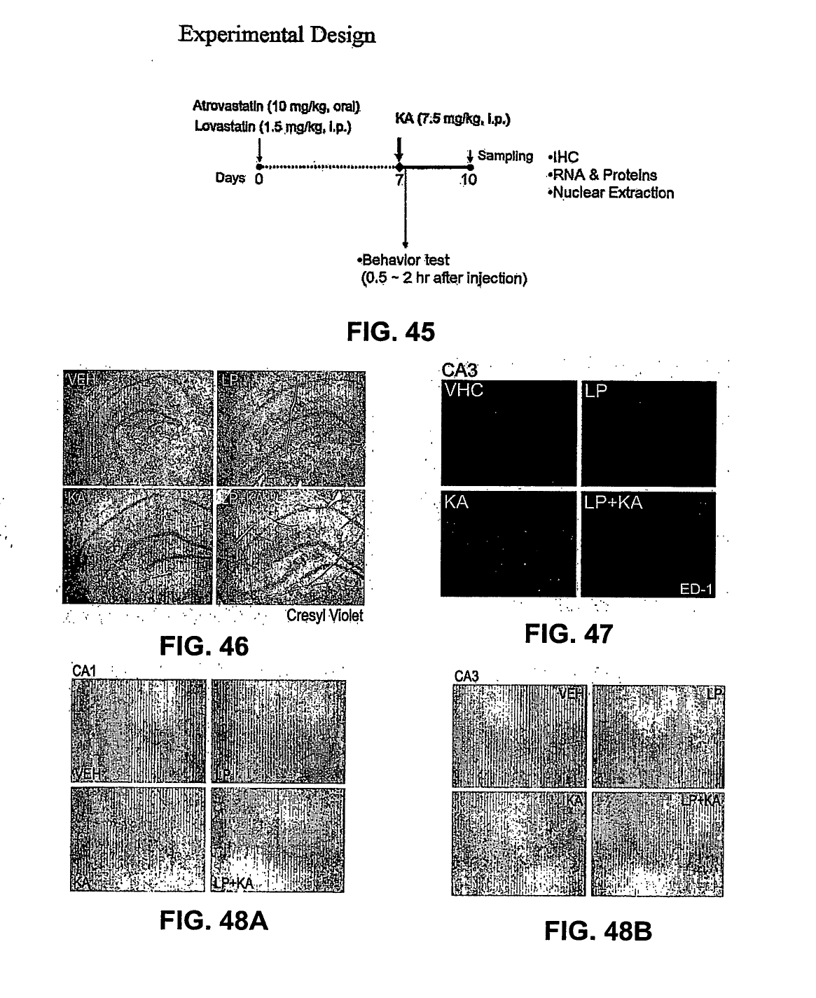

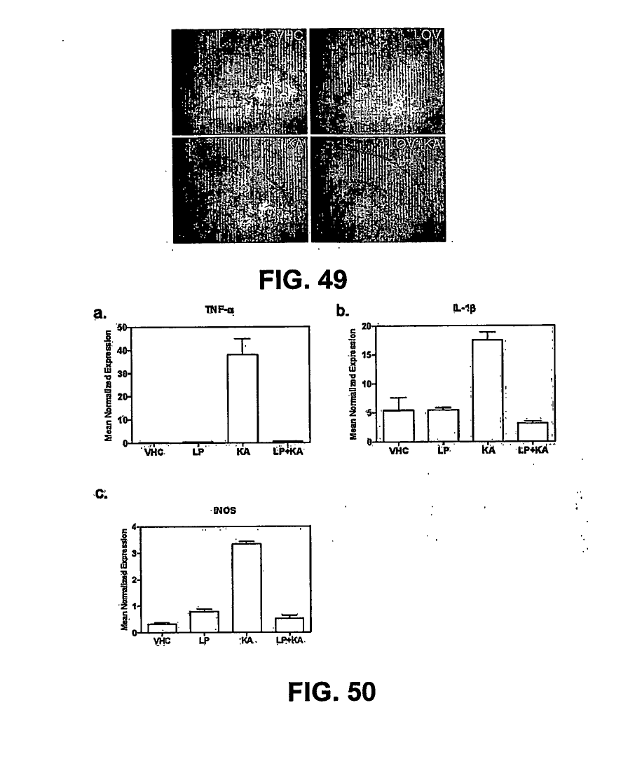

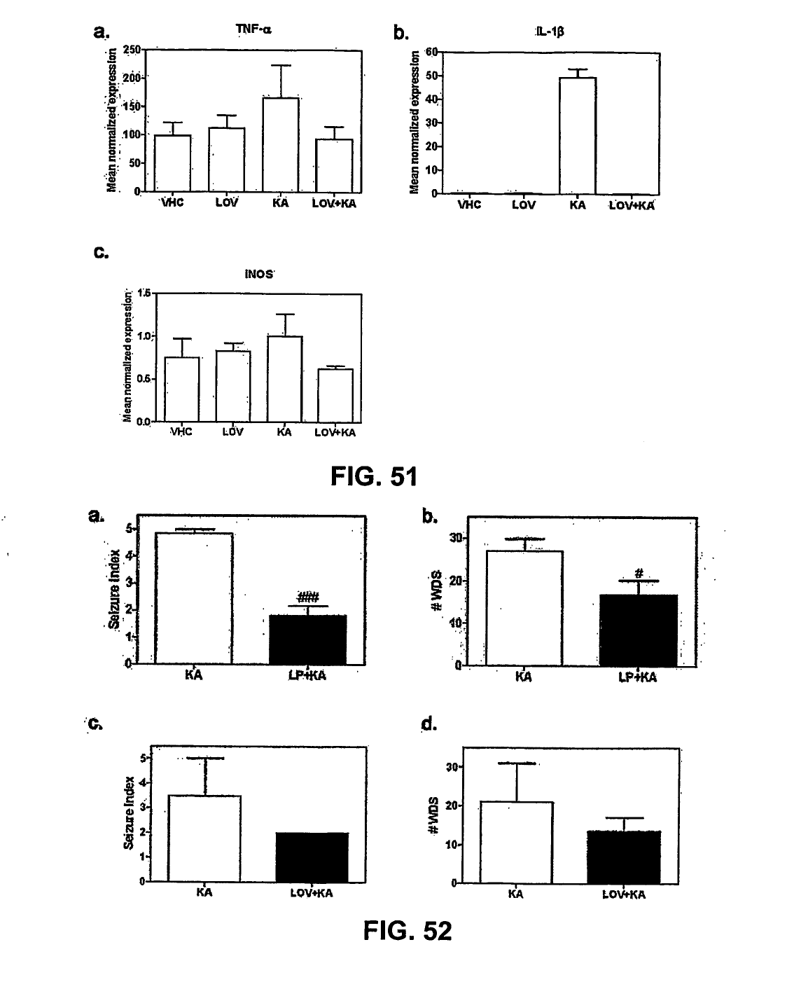

FIGS. 28-52 provide additional data that show the effectiveness of statins in treating a variety of inflammatory diseases such as multiple sclerosis, spinal cord injury, stroke, and kinic acid induced seizures. Additionally, Table 2 provides data concerning the treatment of multiple sclerosis (MS) with statins as compared to two other approved MS drugs. The known drugs that were used were IFN-β and glatiramer acetate (GA) and their effects were compared with the combination of a statin+GSNO in a stroke model.

TABLE 2

| | | | Statin + |
|---|---|---|---|
| Biology | IFN-β | GA | GSNO |
| Antigen presentation | | | reduced |
| Decreased expression of MHCII | Yes | No | Yes |
| Reduced level of co-stimulatory molecules | Yes | No | Yes |
| Inhibition of clonal expression of | Yes | Yes | Yes |
| co-stimulatory molecules | | | |
| Interferences with T-cell activation | Yes | Yes | Yes |
| Decrease inflammatory cytokines | Yes | Yes | Yes |
| Th1 to Th2 deviation | Yes | Yes | Yes |
| Leukocyte trafficking across the blood | Yes | No | Yes |
| brain barrier (BBB) | | | |
| Decrease expression of adhesion | Yes | No | Yes |
| molecules | | | |
| Inhibition of chemokine expression | Yes | No | Yes |
| Inhibition of MMPs | Yes | No | Yes |
| Excludes leukocytes from entering CNS | Yes | No | Yes |
| Protection of BBB | Yes | No | Yes |
| Down regulates the expression of | | | Yes |
| cytokines, iNOS and CNS | | | |
| Antibodies | Neutralizing | Insert | Inert |
| Neuroprotection | Not clear | Yes | Yes |

With respect to spinal cord injuries, a preferred model is the sprague-dawley rats. Injury is induced by dropping a 5 gm weight from a 6 cm height to create approximately a 30 g-cforce therapeutically relevant injury. The extent of the injury and recovery assessed was performed by a 21 point blood brain barrier neurological score. Spinal cord is then extracted and processed for immunocytochemistry and mRNA protein expression. Subsequently, the experimental setup can be described as follows:

[image]

The routs of administration was intraperitoneal (IP) for D-PDMP and gavage for atorvastatin. Spinal cord tissue was extracted from the animal at 1 h, 4 h, 24 h, 48 h, and 1 week following injury. Injured animals were observed for neurological scoring for 15 days. FIGS. 39-42 include data concerning the effect of atorvastatin on spinal cord injury in rats.

All of the compositions and/or methods and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and/or apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Akiyama et al., Alzheimer Dis. Assoc. Disord. 14 Suppl., 1:S47-53, 2000a.
Akiyama et al., Neurobiol. Aging, 21:383-421, 2000b.
Anderson et al., Neurochem. Res., 28:293-305, 2003.
Arai et al., Circ. Res., 82:540-547, 1998.
Arbabi and Maier, Crit. Care Med., 30:S74-S79, 2002.
Bagasra et al, Proc. Natl. Acad. Sci. USA, 92:12041-12045, 1995.
Bal-Price and Brown, J. Neurosci., 21:6480-6491, 2001.
Barber et al., Adv. Neuro., 192:151-164, 2003.
Barone and Feuerstein, J. Cereb. Blood Flow Metab., 19:819-834, 1999.
Bauer et al., Glia, 36:235-243, 2001.
Benveniste et al., Chem. Immunol., 69:31-75, 1997.
Berisha et al., Proc. Natl. Acad. Sci. USA, 91:744-749, 1994.
Berti et al., J. Cereb. Blood Flow Metab., 22:1068-1079, 2002.
Bhunia et al., J. Biol. Chem., 272:15642-15649, 1997.
Bhunia et al, J. Biol. Chem., 273:34349-34357, 1998.
Blazquez et al., FEBS Lett., 489:149-153, 2001.
Blight, Neuroscience, 10:1471-1486, 1983.
Bo et al., Ann. Neurol., 36:778-786, 1994.
Bolanos and Almeida, Biochem. Biophys. Acta, 1411:415-436, 1999.
Boughton-Smith et al., Lancet, 342:338-340, 1993.
Bredesen, Ann. Neurol., 38:839-851, 1995.
Brugg et al., J. Neurochem., 66:733-739, 1996.
Brune et al., Eur. J. Pharmacol., 351:261-272, 1998.
Buisson et al., Br. J. Pharmacol., 106:766-767, 1992.
Bull et al., J. Invest. Derm., 103:435, 1994.
Burney et al., Mutat. Res., 424:37-49, 1999.
Cartier et al., Proc. Natl. Acad. Sci. USA, 92:1674-1678, 1995.
Catani et al., Biochem. Biophys. Res. Commun., 249:275-278, 1998.
Chatterjee, Arterioscler. Thromb. Vasc. Biol., 18:1523-1533, 1998.
Chen and Rosazza, Biochem. Biophys. Res. Commun., 203:1251-1258, 1994.
Chiueh and Rauhala, Free Radic. Res., 31:641-650, 1999.
Chiueh et al. Synapse., 11:346-348, 1992.
Chiueh, Pediatr. Res., 51:414, 2002.
Choi and Lipton, Cell Mol. Life. Sci., 57:1535-1541, 2000.
Chung et al., Biochem. Biophys. Res. Commun., 282:1075-1079, 2001.
Coert et al., J. Neurosurg., 97:914-921, 2002.
Corbett and McDaniel, Methods Enzymol., 268:398-408, 1996.
Corton et al., Eur. J. Biochem, 229:558-565, 1995.
Cross et al., J. Clin. Invest, 93:2684-2690, 1994.
Cui et al., Faseb J., 14:955-967, 2000.
Culmsee et al., J. Mol. Neurosci., 17:45-58, 2001.
Davis et al., Annu. Rev. Pharmacol. Toxicol., 41:203-236, 2001.
Davoli et al., Neuroscience, 115:125136, 2002.
Dawson et al., NIDA Res. Monogr., 136:258-271; discussion 271-253, 1993.
Devlin, J. et al., Transplantation, 58:592-595, 1994.
Dignam et al., Methods Enzymol., 101:582-598, 1983.
Dimagl et al., Trends Neurosci., 22:391-397, 1999.
Ding-Zhou et al., Eur. J. Pharmaco., 1457:137-146, 2002.
Dixon et al., J. Clin. Pharmacol., 31:342-347, 1991.
Eisieik and Leijersfam, Diabetes & Metabolism, 20:116-122, 1994.
Evans et al., Infec. Imm., 60:4133-4139, 1992.
Feigin et al., Lancet Neuro., 12:43-53, 2003.
Fenyk-Melody et al., J. Immunol, 160:2940-2946, 1998.
Fem, Prog. Brain Res., 132:405-411, 2001.
Ford et al., J. Biol. Chem., 277:2430-2436, 2002.
Forste et al, Acta Neuropathol. (Berl), 97:215-220, 1999.
Foster et al., Trends Mol. Med., 9:160-168, 2003.
Gebicke-Haerter, Microsc Res. Tech., 54:47-58, 2001.
Ghosh and Karin, Cell [Suppl.], 109:S81-S96, 2002.
Gilg et al, J. Neuropathol. Exp. Neurol., 59:1063-1069, 2000.
Ginsberg and Busto, Stroke, 20:1627-1642, 1989.
Giri, FASEB J., 16:661-672, 2002.
Giustizieri et al., Am. J. Patho., 1161:1409-1418, 2002.
Goureau et al., Biochem. Biophys. Res. Commun., 186:854-859, 1992.
Green and Chabrier, Drug Discov. Today, 4:4749, 1999.
Green et al., Anal. Biochem., 126:131-138, 1982.
Gruner, J. Neurotrauma, 9:123-126; discussion 126-128, 1992.
Gu et al., Science, 297:1186-1190, 2002.
Haga et al., Brain Res., 601:88-94, 1993.
Hakomori et al., Methods Enzymol., 363:191-207, 2003.
Hallenbeck, Nat. Med., 8:1363-1368, 2002.
Hamid et al., Lancet, 342:1510-1513, 1993.
Han et al., J. Cereb. Blood Flow Metab., 23:589-598, 2002.
Hannun, J. Biol. Chem., 269:3125-3128, 1994.
Haq et al., J. Neurochem., 86:1428-1440, 2003.
Hardie and Carling, Eur. J. Biochem., 246:259-273, 1997.
Hardie et al., Annu. Rev. Biochem., 67:821-855, 1998.
Hardie, Biochim. Biophys. Acta, 1123:231-238, 1992.
Hardy et al., J. Immunol., 153:1754-1761, 1994.
Hashmi et al., FEBS Lett. 196:247-250, 1986.
Hawley et al., J. Biol. Chem., 271:27879-27887, 1996.
Hays, Curr. Pharm. Des., 4:335-348, 1998.
Herrmann et al., J. Biol. Chem., 270:2901-2905, 1995.
Hesse et al., Surg. Gynecol. Obstet., 166:147-153, 1988.
Hogg, Annu. Rev. Pharmacol. Toxico1. 42:585-600, 2002.
Hogg, Free Radic. Biol. Med., 28:1478-1486, 2000.
Hooper et al, Proc. Natl. Acad. Sci. USA, 94:2528-2533, 1997.
Hooper et al, Proc. Natl. Acad. Sci. USA, 95:675-680, 1998.
Hu H M et al., J. Biol. Chem., 275:16373-16381, 2000.
Huang et al., J. Cereb. Blood Flow Metab., 16:981-987, 1996.
Huang et al., Science, 265:1883-1885, 1994.
Iadecola et al., J. Cereb. Blood Flow Metab., 15:378-384, 1995.
Iadecola, Trends Neurosci., 20:132-139, 1997.
Issazadeh et al., J. Neurosci. Res., 40:579-590, 1995.
Iwabuchi and Nagaoka, Blood, 100:1454-1464, 2002.
Janero, Free Radic. Biol. Med. 28:1495-1506, 2000.
Johnson et al., Cardiovasc. Surg., 6:367-372, 1998.
Joshi et al., Res. Commun. Mol. Pathol. Pharmacol. Mar., 91:339-346, 1996.
Jourd'heui et al., J. Biol. Chem., 278:15720-15726, 2003.
Kametsu et al., J. Cereb. Blood Flow Metab., 23:416-422, 2003.
Kanety et al., J. Biol. Chem., 270:23780-23784, 1995.
Kaposzta et al., Circulation, 105:1480-1484, 2002b.
Kaposzta et al, Circulation, 106:3057-3062, 2002a.
Kato et al, Brain Res., 734:203-212, 1996.
Kaurs and Halliwell, FEBS Lett., 350:9-12, 1994.
Keinanen et al, Gene, 234:297-305, 1999.
Kemp et al, Trends Biochem. Sci., 24:22-25, 1999.
Khan et al., J. Endotoxin Res., 6:41-50, 2000.
Khan et al., J. Neurochem., 71:78-87, 1998.
Kharitonov et al, Lancet, 343:133-135, 1994.
Kiernan, Trends Pharmacol. Sci., 11:316, 1990.
Kim et al., Circ. Res., 84:253-256, 1999.
Kim et al., Int. Immunopharmacol., 1:1421-1441, 2001.
Kim et al., J. Biol. Chem., 276:19102-19110, 2001.
Kim et al., J. Biol. Chem., 277:40594-40601, 2002.
Klinkert et al., J. Neuroimmunol., 72(2)163-168, 1997.
Kluge et al., J. Neurochem., 69:2599-2607, 1997.
Kolb-Bachofen et al., Lancet., 344(8915):139, 1994.
Kolesnick et al., Biochem. Cell Biol, 72:471-474, 1994.
Koprowski et al., Proc. Natl. Acad. Sci. USA, 90:3024-3027, 1993.
Kroncke et al., Biochem. Biophys. Res. Commun., 175:752-758, 1991.
Kwak et al., Nat. Med., 6:1399-1402, 2000.
Langford et al., Lancet, 344:1458-1460, 1994.
Lassmann and Wisniewski, Arch. Neurol., 36:490-497, 1979.
Lazo et al., Proc. Natl. Aced. Sci. USA, 85:7647-7651, 1988.
Lee et al., NeuroReport, 3:841-844, 1992.
Leist et al., Eur. J. Neurosci., 9:1488-1498, 1997.
Leker et al., Brain Res. Brain Res. Rev., 39:55-73, 2002.
Li and Forstermann, J. Pathol., 190:244-254, 2000.
Linardic et al., Cell Growth Differ., 7:765-774, 1996.
Lipton et al., Nature, 413:171-174, 2001.
Lipton et al., Trends Neurosci., 25:474-480, 2002.
Lipton, Nature, 413:118-119, 121, 2001.
Liu et al., J. Biol. Chem., 274:1140-1146, 1999.
Liu et al., J. Neurosci., 17:5395-5406, 1997.
Liu et al., Trends Neurosci., 24:581-588, 2001.
Love, Neuropsychopharmacol. Biol. Psychiatry, 27:267-282, 2003.
Lowick et al., J. Clin. Invest., 93:1465-1472, 1994.
Maimone et al, J. Neuro Immunol., 32:67-74, 1991.
Mandia et al., Invest. Opthalmol., 35:3673-3689, 1994.
Mannick et al., J. Cell Biol., 154:1111-1116, 2001.
Mannick et al., Science, 284:651-654, 1999.
Marcus et al., Glia, 41:152-160, 2003.
Marshall and Stamler, Biochemistry, 40:1688-1693, 2001.
Marshall and Stamler, J. Biol. Chem., 277:34223-34228, 2002.
Matsuyama et al., J Spinal Disord., 11:248-252, 1998.
Mattson and Camandola, J. Clin. Invest., 107:247-254, 2001.
McCarthy and de Vellis, J. Cell Biol., 85:890-902, 1980.
McGuiness et al, J. Neuroimmunol., 75:174-182, 1997.
McGuinness et al, J. Neuroimmunol., 61:161-169, 1995.
Megson, Drugs of the Future, 25:701-715, 2000.
Meller et al., Europ. J. Pharmacol., 214:93-96, 1992.
Merrill and Benveniste, Trends Neurosci., 19:331-338, 1996.
Merrill et al., J. Immunol., 151:2132-2141, 1993.
Miller et al, J. Pharmacol. Exp. Ther., 264:11-16, 1993.
Miller et al., Lancet, 34:465-466, 1993.
Mitrovic et al., Neurosci., 61:575-585, 1994.
Mohr et al., Biochem. Biophys Res. Commun., 238:387-391, 1997.
Molloy et al, Circulation, 98:1372-1375, 1998.
Moore et al, Brit. J. Pharmacol, 102:198-202, 1991.
Moore et al, Brit. J. Pharmacol., 108:296-297, 1992.
Moore et al., Eur. J. Biochem., 199:691-697, 1991.
Moser et al, Ann. Neurol., 16:628-641, 1984.
Moser et al., In: The metabolic and molecular bases of inherited disease, 7th ed., Scriver et al (Eds.), NY, McGraw-Hill, 2:232549, 1995.
Moser, J. Neuropathol. Expt. Neurol., 54:740-744, 1995.
Mosser et al., Nature, 361:726-730, 1993.
Muhl et al., Br. J. Pharmcol., 112:1-8, 1994.
Mulligan et al, Br. J. Pharmacol., 107:1159-1162, 1992.
Nagafuji et al., Neurosci., 147:159-162, 1992.
Namura et al., J. Neurosci., 18:3659-3668, 1998.
Nunokawa et al., Biochem. Biophys. Res. Commun., 223:347-352, 1996.
Olesen et al., Trends Pharmacol. Sci., 15:149-153, 1994.
Page et al., Adv. Exp. Med. Biol., 370:353-356, 1994.
Pahan et al., J. Biol. Chem., 273:2591-2600, 1998.
Pahan et al., J. Clin. Invest, 100:2671-2679, 1997.
Pahan et al., J. Neurochem., 73:513-520, 1999.
Pahan et al., J. Neurochem., 74:2288-2295, 2000.
Petros et al., Lancet., 338(8782-8783):1557-1558, 1991.
Pitossi et al., J. Neurosci. Res., 48:287-298, 1997.
Poli, J. Biol. Chem., 273:29279-29282, 1998.
Powers et al., J Neuropathol. Expt. Neurol., 51:630-643, 1992.
Powers et al, J. Neuropathol. Expt. Neurol., 51:630-643, 1992.
Powers, J. Neuropathol. Expt Neurol., 54:710-719, 1995.
Rauhala et al., Faseb J., 12:165-173, 1998.
Remington's Pharmaceutical Sciences, 18th Ed. Mack Printing Company, 1990.
Richardson, Clinical Science, 102:99-105, 2002.
Rizzo et al., Neurology, 36:357-361, 1986.
Rizzo et al., Neurology, 39:1415-1422, 1989.
Rodríguez et al., Proc. Natl. Acad. Sci. USA, 100:336-341, 2003.
Rogers et al, Proc. Natl. Acad. Sc. USA, 89(21):10016-10020, 1992.
Rothwell, Brain Behav. Immun., 17:152-157, 2003.
Rudick and Ransohoff, Arch. Neurol, 49:265-270, 1992.
Ruuls et al., Clin. Exp. Immunol., 103:467-474, 1996.
Ruzicka et al, J. Invest. Derm., 103:397, 1994.
Saliba and Henrot, Biol. Neonate., 79:224-227, 2001.
Salom et al, Brain Res., 865:149-156, 2000.
Salt et al., Biochem, J., 334:177-187, 1998.
Sambrook et al., In: Molecular cloning, Cold Spring Harbor Laboratory Press, Cold Spring Harbor, N.Y., 2001.
Sarti et al., Stroke, 31:1588-1601, 2000.
Satake et al., Brain Res. Mol. Brain. Res., 85:114-122, 2000.
Schilling et al., Intensive Care Med., 19(4):227-231, 1993.
Schrammel et al., Free Radic. Biol. Med., 34:1078-1088, 2003.
Sehba et al., Stroke, 30:1955-1961, 1999.
Sekhon et al., Brain Res., 971:1-8, 2003.
Sekhon et al., J. Neurochem., 81(Suppl. 1):103, 2002.
Sequeira et al., Nitric Oxide, 1:315-329, 1997.
Simmons and Murphy, J. Neurochem., 59:897-905, 1992.
Singh et al., J. Biol. Chem., 273:20354-20362, 1998.
Singh, Mol. Cell. Biochem., 167:1-29, 1997.
Smith et al., Brain Pathol., 9:69-92, 1999.
Spiegel and Merrill, Faseb. J., 10:1388-1397, 1996.
Spiegel and Milstien, FEBS Lett., 476:55-57, 2000.
Sprenkle et al., FEBS Lett., 403:254-258, 1997.
Stadler et al., J. Immunol., 147:3915-3920, 1991.
Stamler et al., Neuron., 18:691-696, 1997.
Stanislaus et al., J. Neurosci. Res., 66:155-162, 2001.
Stefanelli et al., Biochem. Biophys. Res. Commun., 243:821-826, 1998.
Steffen et al., Biochem. J., 356:395-402, 2001.
Stein et al., Biochem. J., 345:437-443, 2000.
Stevens et al., Brain Res., 932:110-119, 2002.
Stewart and Heales, Free Radic. Biol. Med., 34:287-303, 2003.
Stoll et al., Prog. Neurobiol., 56:149-171, 1998.
Su et al., J. Neuroimmunol, 134:52-60, 2003.
Sugimoto and Iadecola, Neurosci. Lett., 331:25-28, 2002.
Sullivan et al., FEBS Leff., 353:33-36.
Suzuki et al., Brain Res., 951:113-120, 2002.
Suzuki et al., Naunyn Schmiedebergs Arch Pharmacol., 363:94-100, 2001.
Taupin, et al., Eur. J. Immunol., 27:905-913, 1997.
Taylor et al., J. Biol. Chem., 273:15148-15156, 1998.
Thiemermann and Vane, Br. J. Pharmacol., 114(6):1273-1281, 1995.
Thompson, Science, 267:1456-1462, 1995.
Torreilles et al., Brain. Res. Brain. Res., 30:153-163, 1999.
Trifiletti et al., Europ. J. Pharmacol., 218:197-198, 1992.
Tschape et al., EMBO, 21:6367-6376, 2002.
Tseng et al., J. Pharmacol. Exp. Ther., 292:737-742, 2000.
Tsukada et al., J. Neurol. Sci., 104:230-234, 1991.
Turnley et al., J. Neurochem., 72:1707-1716, 1999.
van der Veen et al., J. Neuroimmunol., 77:1-7, 1997.
Villarroya et al., J. Neuroimmunol., 64:55-61, 1996.
Vincent et al., Diabetes, 40:1259-1266, 1991.
Viollet et al., J. Clin. Invest, 111:91, 2003.
Virag et al., Toxicol. Lett., 140-141:113-124, 2003.
Vodovotz et al., Int J. Radiat. Oncol. Biol. Phys., 48:1167-1174, 2000.
Vodovotz et al., J. Exp. Med., 184:1425-1433, 1996.
Wada et al., J. Neurosurg., 89:807-818, 1998a.
Wada et al., Neurosurgery, 43:1427-1436, 1998b.
Waldmeier, Prog. Neuropsychopharmacol. Biol. Psychiatry, 27:303-321, 2003.
Welsh et al., Endocrinol., 129:3167-3173, 1991.
White et al., Proc. Natl., Acad. Sci. USA, 91:1044-1048, 1994.
Wiesner and Dawson, J. Neurochem., 66:1418-1425, 1996.
Willmot smf Bath, Expert Opin. Investig. Drugs, 12:455-470, 2003.
Winder and Hardie, Am. J. Physiol., 277:E1, 1999.
Winlaw et al., Lancet, 344:373-374, 1994.
Won et al., Brain Res., 903:207-215, 2001.
Xi X, and Zhang, J. Biol. Chem., 276:41029-41034.
Xie et al., J. Biol. Chem., 269:4705-4708, 1994.
Yang et al., J. Biol. Chem., 276:38341-38344, 2001.
Yao et al., Brain Res. Mol. Brain. Res., 91:112-118, 2001.
Yeh et al., J. Vasc. Res., 38:551-559, 2001.
Young, J. Emerg. Med. 11 Suppl., 1:13-22, 1993.
Zagariya et al., Mol. Cell. Biol., 18:2815-2824, 1998.
Zembowicz and Vane, Proc. Natl. Acad. Sci. USA, 89:2051-2055, 1992.
Zeng et al., Am. J. Physiol. Heart Circ. Physiol., 281:H432-439, 2001.
Zhang et al., Biochem. Pharmacol., 55:1873-1880, 1998.
Zhang et al., J. Cereb. Blood Flow Metab., 14:217-226, 1994a.
Zhang et al., Science, 263:687-689, 1994b.
Zhao et al., Brain Res., 872:215-218, 2000.
Zhao et al., Brain Res., 966:308-311, 2003.
Zhu et al., Life Sci., 71:1985-1996, 2002.
Zielasek et al., Cell Immunol., 141:111-120, 1992.

1. A method of preventing or treating an inflammatory disease or condition in a patient comprising administering to the patient a therapeutically effective amount of:
(a) a glutathione donor; and
(b) 5-amino 4-imidazolecarboxamide ribotide(AICAR), a 3-hydroxy-3-methylgluatryl-coenzymeA (HMG-CoA) reductase inhibitor, D-threo-1-Phenyl-2-decanoylamino-3-morpholino-1-propanol HCl (D-PDMP), or 1,5-(butylimino)-1, 5-dideoxy-D-glucitol (Miglustat), or a derivative thereof.

2. The method of claim 1, further comprising determining a patient is in need of the prevention or treatment.

3. The method of claim 2, wherein determining a patient in need of the prevention or treatment comprises determining whether a patient is at risk for developing an inflammatory disease or condition.

4. The method of claim 3, wherein determining whether a patient is at risk for developing an inflammatory disease or condition comprises taking a family history or a patient history.

5. The method of claim 1, wherein the glutathione donor is formulated in a pharmaceutical acceptable vehicle.

6. The method of claim 1, wherein AICAR, the HMG-CoA reductase inhibitor, D-PDMP, or Miglustat is formulated in a pharmaceutical acceptable vehicle.

7. The method of claim 1, wherein the glutathione donor is administered to the patient before, during, or after AICAR, an HMG-CoA reductase inhibitor, D-PDMP, or Miglustat is administered to the patient.

8. The method of claim 1, wherein AICAR, an HMG-CoA reductase inhibitor, D-PDMP, or Miglustat is administered to the patient before, during, or after the glutathione donor is administered to the patient.

9. The method of claim 1, wherein the glutathione donor is administered to the patient before, during, and after AICAR, an HMG-CoA reductase inhibitor, D-PDMP, or Miglustat is administered to the patient.

10. The method of claim 1, wherein AICAR, an HMG-CoA reductase inhibitor, D-PDMP, or Miglustat is administered to the patient before, during, and after the glutathione donor is administered to the patient.

11. The method of claim 1, wherein the glutathione donor is a molecule that comprises glutathione.

12. The method of claim 1, wherein the glutathione donor is a precursor molecule to glutathione.

13. The method of claim 1, wherein the glutathione donor is S-nitroglutathione (GSNO), L-2-oxo-thiazolidine 4-carboxylate (Procysteine), N-acetyl cysteine (NAC), or N-acetyl glutathione.

14. The method of claim 13, wherein the glutathione donor is S-nitroglutathione (GSNO).

15. The method of claim 1, wherein the glutathione donor and AICAR are administered to the patient.

16. The method of claim 1, wherein the glutathione donor and an HMG-CoA reductase inhibitor are administered to the patient.

17. The method of claim 16, wherein the HMG-CoA reductase inhibitor is a statin.

18. The method of claim 17, wherein the statin is atorvastatin, lovastatin, rosuvastatin, fluvastatin, pravastatin, simvastatin, or cerivastatin.

19. The method of claim 18, wherein the statin is atorvastatin.

20. The method of claim 1, wherein the glutathione donor and D-PDMP are administered to the patient.

21. The method of claim 1, wherein the glutathione donor and Miglustat are administered to the patient.

22. The method of claim 1, wherein the glutathione donor, AICAR, an HMG-CoA reductase inhibitor, D-PDMP, and Miglustat are administered to the patient.

23. The method of claim 1, wherein the inflammatory disease or condition is stroke, X-adenoleukodystrophy (X-ALD), cancer, septic shock, adult respiratory distress syndrome, myocarditis, arthritis, an autoimmune disease, an inflammatory bowel disease, an inflammatory nervous system disease, an inflammatory lung disorder, an inflammatory eye disorder, a chronic inflammatory gum disorder, a chronic inflammatory joint disorder, a skin disorder, a bone disease, a heart disease, kidney failure, a chronic demyelinating disease, an endothelial cell disease, a cardiovascular disease, obesity, a common cold, lupus, sickle cell anemia, diabetes, or a neurodegenerative disease.

24. The method of claim 23, wherein the inflammatory disease or condition is diabetes.

25. The method of claim 23, wherein the inflammatory disease or condition is stroke or a neurodegenerative disease.

26. The method of claim 25, wherein the neurodegenerative disease is Alzheimer's disease, Parkinson's disease, Landry-Guillain-Barre-Strohl syndrome, multiple sclerosis, viral encephalitis, acquired immunodeficiency disease(AIDS)-related dementia, amyotrophic lateral sclerosis, brain trauma, or a spinal cord disorder.

27. The method of claim 1, further comprising administering a second therapy used to treat or prevent an inflammatory disease or condition.

28. The method of claim 1, wherein the glutathione donor is comprised in a pharmaceutically acceptable composition.

29. The method of claim 1, wherein the AICAR, the HMG-CoA reductase inhibitor, the D-PDMP, or the Miglustat is comprised in a pharmaceutical acceptable composition.

30. The method of claim 1, wherein the glutathione donor and the AICAR, the HMG-CoA reductase inhibitor, the D-PDMP, or the Miglustat, are comprised in separate compositions.

31. The method of claim 1, wherein the glutathione donor and the AICAR, the HMG-CoA reductase inhibitor, the D-PDMP, or the Miglustat, are comprised in the same composition.

32. The method of claim 1, wherein the glutathione donor is not GSNO.

33. A composition comprising:
(a) a glutathione donor; and
(b) 5-amino 4-imidazolecarboxamide ribotide (AICAR), a 3-hydroxy-3-methylgluatryl-coenzymeA (HMG-CoA) reductase inhibitor, D-threo-1-Phenyl-2-decanoylamino-3-morpholino-1-propanol HCl (D-PDMP), or 1,5-(butylimino)-1,5-dideoxy-D-glucitol (Miglustat), or a derivative thereof.

34. The composition of claim 33, further defined as a pharmaceutically acceptable composition.

35. The composition of claim 33, wherein the glutathione donor and the AICAR, the HMG-CoA reductase inhibitor, the D-PDMP, or the Miglustat are formulated in a pharmaceutically acceptable vehicle.

36. The composition of claim 33, wherein the glutathione donor is S-nitroglutathione (GSNO), L-2-oxo-thiazolidine 4-carboxylate (Procysteine), N-acetyl cysteine (NAC), or N-acetyl glutathione.

37. The composition of claim 36, wherein the glutathione donor is S-nitroglutathione (GSNO).

38. The composition of claim 33, wherein the composition comprises a glutathione donor and AICAR.

39. The composition of claim 33, wherein the composition comprises a glutathione donor and an HMG-CoA reductase inhibitor.

40. The composition of claim 39, wherein the HMG-CoA reductase inhibitor is a statin.

41. The composition of claim 40, wherein the statin is atorvastatin, lovastatin, rosuvastatin, fluvastatin, pravastatin, simvastatin, or cerivastatin.

42. The composition of claim 41, wherein the statin is atorvastatin.

43. The composition of claim 33, wherein the composition comprises a glutathione donor and D-PDMP.

44. The composition of claim 33, wherein the composition comprises a glutathione donor and Miglustat.

45. The composition of claim 33, wherein the composition comprises a glutathione donor, AICAR, an HMG-CoA reductase inhibitor, D-PDMP, and Miglustat.

46. The composition of claim 33, wherein the glutathione donor is not GSNO.

47. (canceled)

48. A pharmaceutically acceptable composition comprising a glutathione donor and a statin, or derivatives thereof.

49. The pharmaceutical acceptable composition of claim 48, wherein the glutathione donor is S-nitroglutathione(GSNO), L-2-oxo-thiazolidine 4-carboxylate (Procysteine), N-acetyl cysteine (NAC), or N-acetyl glutathione.

50. The pharmaceutically acceptable composition of claim 49, wherein the glutathione donor is S-nitroglutathione (GSNO).

51. The pharmaceutically acceptable composition of claim 50, wherein the statin is atorvastatin, lovastatin, rosuvastatin, fluvastatin, pravastatin, simvastatin, or cerivastatin.

52. The pharmaceutical acceptable composition of claim 51, wherein the statin is atorvastatin.

53. The pharmaceutically acceptable composition of claim 48, wherein the glutathione donor is L-2-oxo-thiazolidine 4-carboxylate (Procysteine), N-acetyl cysteine (NAC), or N-acetyl glutathione.

54. The pharmaceutically acceptable composition, wherein the glutathione donor is not GSNO.

55. A method of preventing or treating diabetes in a patient comprising administering to the patient the composition of claim 33.

56. The method of claim 55, wherein the diabetes is type 1 diabetes.

57. The method of claim 55, wherein the glutathione donor is S-nitroglutathione (GSNO).

58. A method of preventing or treating diabetes in a patient comprising administering to the patient the pharmaceutically acceptable composition of claim 48.

59. The method of claim 58, wherein the diabetes is type 1 diabetes.

60. The method of claim 58, wherein the glutathione donor is S-nitroglutathione (GSNO).

61. The method of claim 58, wherein the statin is atorvastatin, lovastatin, rosuvastatin, fluvastatin, pravastatin, simvastatin, or cerivastatin.
